(19) United States
(12) Patent Application Publication
(10) Pub. No.: US 2009/0155230 A1
(43) Pub. Date: Jun. 18, 2009

(54) Novel genes and markers in essential arterial hypertension

(75) Inventors: Jukka T. Salonen, Kuopio (FI); Juha-Matti Aalto, Siilinjarvi (FI); Pekka Uimari, Kuopio (FI); Mia Pirskanen, Kuopio (FI)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 11/822,412

(22) Filed: Jul. 5, 2007

Related U.S. Application Data
(60) Provisional application No. 60/819,014, filed on Jul. 7, 2006.
(60) Provisional application No. 60/867,454, filed on Nov. 28, 2006.

(51) Int. Cl. A61K 48/00; G01N 33/68; C12Q 1/68; C12Q 1/52; G01N 33/53; A61K 38/02; A61K 31/7088; A61K 39/395

(52) U.S. Cl. 424/93.21; 436/86; 435/6; 435/16; 436/501; 435/7.1; 514/2; 424/139.1; 514/44

(57) ABSTRACT

The present invention relates to previously unknown disease associations between various genes, loci and biomarkers and essential hypertension. The detection of these biomarkers provides novel in vitro methods and test kits which can be used as an aid when making risk assessment, molecular diagnosis or prognosis of HT or a HT related condition. The disclosed methods and test kits do not require interaction with the body of a subject during the biomarker detection. Instead the methods and test kits are for in vitro use (e.g. in a clinical laboratory) and typically biological samples for the biomarker analyses using a method or a test kit of this invention have been collected earlier in a different place. In addition the biomarkers provide methods and systems for identifying novel agents for preventing, treating and/or reducing risk of HT or a HT related condition. The HT associated genes can be used to develop novel therapies for prevention and/or treatment of essential hypertension.

### RELATED APPLICATIONS

This application claims the benefit of U.S. provisional Application No. 60/819,014, filed on Jul. 7, 2006 and U.S. provisional Application No. 60/867,454 filed on Nov. 28, 2006. The entire teachings of the above applications are incorporated herein by reference.

### BACKGROUND OF THE INVENTION

Cardiovascular Diseases (CVD) (ICD/10 codes I00-I99, Q20-Q28) include ischemic (coronary) heart disease (IHD, CHD), hypertensive diseases, cerebrovascular disease (stroke) and rheumatic fever/rheumatic heart disease, among others. Essential hypertension (HT; ICD/10 codes I10-I15) is defined as blood pressure measurements of 140/90 mmHg or greater without any obvious cause such as renal disease, adrenal tumor, or drug therapy constitutes about 95% of all hypertension cases. HT prevalence rises with age irrespective of the type of BP measurement and the operational thresholds used for diagnosis. The prevalence of elevated blood pressure is 20-30% of the adult population in most western countries. HT aggregates with other cardiovascular risk factors such as abdominal obesity, dyslipidaemia, glucose intolerance, hyperinsulinaemia and hyperuricaemia, possibly because of a common underlying cause. Apart from being a CVD itself, HT is a risk factor for other CVD, such as IHD, stroke and congestive heart failure (CHF). About half of people having their first heart attack and two thirds of people having their first stroke, have blood pressure (BP) higher than 160/95 mmHg. HT precedes the development of CHF in 91% of cases. (AHA, 2004).

The pressure required to move blood through the circulatory bed is provided by the pumping action of the heart [cardiac output (CO)] and the tone of the arteries [peripheral resistance (PR)]. Each of these primary determinants of BP is, in turn, determined by the interaction of a complex series of factors. Data generated from animal models, human twin and family studies suggest that approximately 30 to 60% of blood pressure arises from genetic factors according to recent review (Binder A, 2007). It seems that hypertension cannot be understood without appreciating the critical role of gene-environment interactions as evidenced by cross-cultural population studies (Weder A B, 2007). Nuclear family studies show greater similarity in BP within families than between families, with heritability estimates ranging between 0.20 and 0.46. Twin studies document greater concordance of BP in monozygotic than dizygotic twins, giving the highest heritability estimates between 0.48 and 0.64. Adoption studies demonstrate greater concordance of BP among biological siblings than adoptive siblings living in the same household, estimating heritability between 0.45 and 0.61. (Fuentes R M, 2003).

In the rare Mendelian forms of high and low BP single genes can have major effects on BP (Lifton R P et al, 2001, Luft F C, 2003). Although identifiable single-gene mutations account for only a small percentage of all HT cases, study of these rare Mendelian disorders has been used to elucidate pathophysiologic mechanisms that predispose to more common forms of HT and to suggest novel therapeutic approaches. Several mutations that cause Mendelian forms of human HT or hypotension have been described to date (Lifton R P et al, 2001, Luft F C, 2003). These mutations affect BP by altering renal salt handling, reinforcing the hypothesis that a major component in the development of HT depends on genetically determined renal dysfunction with resultant salt and water retention (Guyton A C, 1991). Importantly, all the monogenic HT syndromes identified were caused by defects resulting in renal salt retention, whereas all the low BP syndromes shared a common mechanism of excess renal sodium loss (Hopkins P N and Hunt S C, 2003). The best studied monogenic cause of HT is the Liddle syndrome, a rare but clinically important disorder in which constitutive activation of the epithelial sodium channel predisposes to severe, treatment-resistant HT (Shimkets R A et al, 1994). Epithelial sodium channel activation has been traced to mutations in the beta or gamma subunits of the channel, resulting in inappropriate sodium retention at the renal collecting duct level. Patients with the Liddle syndrome typically present with volume-dependent, low-renin, and low-aldosterone HT.

Candidate gene studies have concerned genes encoding components of the renin-angiotensin-aldosterone system, the epithelial sodium channel, adrenergic receptors, G protein subunits, oxidative stress and other cellular signaling mediators and modifiers. Thus far, the candidate gene approach has provided more examples than the linkage approach of gene variants that appear to affect BP. Reasonable candidate genes to consider include genes related to physiological systems known to be involved in the control of BP and genes known to affect BP in mouse models. To date more than 80 candidate genes have been evaluated for HT. However, the association with HT of only a few genes have been widely replicated: angiotensinogen precursor (AGT), adducin 1 (ADD1) and guanine nucleotide-binding protein, beta-3 subunit (GNB3) (Hopkins PN and Hunt S C, 2003). In addition recently the impact of endothelial NO synthase gene (NOS3) polymorphism on the development of HT was confirmed by a large meta-analysis which included 35 genetic association studies (Zintzaras E et al, 2006). New HT candidate genes, such as cytochrome b-245, alpha polypeptide (CYBA), emerge together with the growing amount of knowledge about HT pathophysiology (Kokubo Y et al, 2005; Moreno M U et al, 2006). Gene-environment interactions affecting HT treatment have been shown between AGT, ADD1 and salt intake reduction (Hunt S C et al, 1998; Hunt S C et al, 1999; Cusi D et al, 1997), and between ADD1, GNB3 and diuretic treatment (Cusi D et al, 1997; Turner S T et al, 2001). Gene-gene interactions affecting HT risk development have been shown between ADD1 and the ACE gene I/D polymorphisms and between serotonin 2 (5-HT2) and endothelin-1 (ET-1) genes (Staessen J A et al, 2001; Yamamoto M et al. 2006). Lessons teamed from the studies of candidate genes to date include the shortcomings that result from limited statistical power of many studies, expected variation from one population to another, the need for better phenotyping of study subjects, the relatively small effect of the genes studied on population prevalence of HT, and the lack of sufficient certainty of consequences of any genes studied thus far to make treatment recommendations based on genotype (Hopkins P N and Hunt S C, 2003).

So far 25 genome-wide scanning studies have reported significant or suggestive linkage for BP/IT (Binder A, 2007). Some scans have utilized families, others affected or dissimilar sibling pairs. Linked loci with at least suggestive LOD scores have been observed on every chromosome. Perhaps most striking is the lack of consistency among the linked loci. Koivukoski et al, 2004 applying the genome-search meta-analysis method (GSMA) to nine published genome-wide scans of BP (n=5) and HT (n=4) in Caucasian populations found evidence of susceptibility regions for BP/HT only on chromosomes 2p12-q22.1 and 3p14.1-q12.3, which had modest or non-significant linkage in each individual study. This may serve to illustrate the heterogeneity of human HT as well as the potential shortcomings of family-based linkage studies.

Essential hypertension (HT) affects over one billion people worldwide, 20-55% of middle age Americans (over 50 million people) and Europeans (over 200 million people) across various ethnic subgroups, making it a public health issue of considerable magnitude and the single greatest risk factor for diseases of the brain, heart, and kidneys. Hypertension is the number one reason adults go to the doctor. It is estimated that Americans spend more than $8 billion per year on blood pressure medications. Even so, only 27% of Americans with high blood pressure have adequate blood pressure control.

Death and illness from diseases associated with high blood pressure exceeds that from all other causes and costs more than $250 billion each year. It is known that essential HT aggregates with major cardiovascular risk factors such as abdominal obesity, dyslipidaemia, glucose intolerance, hyperinsulinaemia and hyperuricaemia, possibly because of a common underlying cause and is a risk factor for other CVD, such as stroke and congestive heart failure (CHF). In 2001 an estimated 16.6 million—or one-third of total global deaths—resulted from the various forms of CVD (7.2 million due to HT, 5.5 million to cerebrovascular disease, and an additional 3.9 million to hypertensive and other heart conditions). At least 20 million people survive heart attacks and strokes every year, a significant proportion of them requiring costly clinical care, putting a huge burden on long-term care resources.

The high prevalence of essential HT in adult population and it's significant contribution to morbidity and mortality from cardiovascular diseases shows unmet medical need both for diagnostic methods to identify subjects having increased risk essential hypertension and for better therapies to prevent and to treat HT. The present invention provides a number of new correlations between various polymorphic alleles and essential hypertension. The HT associated polymorphic alleles, genes and loci disclosed in this invention provide the basis for improved risk assessment, more detailed diagnosis and prognosis of essential HT, and for the development of novel therapies to prevent and treat essential hypertension or related condition.

### SUMMARY OF THE INVENTION

The present invention relates to previously unknown disease associations between various genes, loci and biomarkers and essential hypertension. The detection of these biomarkers provides novel in vitro methods and test kits which can be used as an aid when making risk assessment, molecular diagnosis or prognosis of HT or a HT related condition. The disclosed methods and test kits do not require interaction with the body of a subject during the biomarker detection. Instead the methods and test kits are for in vitro use (e.g. in a clinical laboratory) and typically biological samples for the biomarker analyses using a method or a test kit of this invention have been collected earlier in a different place. In addition the biomarkers provide methods and systems for identifying novel agents for preventing, treating and/or reducing risk of HT or a HT related condition. The HT associated genes can be used to develop novel therapies for prevention and/or treatment of essential hypertension.

Accordingly in a first aspect, the present invention provides methods and kits for determining in vitro a susceptibility to HT or a HT related condition in an individual. The methods comprise the step of detecting from a biological sample one or more HT associated biomarkers, wherein the biomarkers are related either to one or more genes set forth in table 1, and/or are selected from the SNP markers listed in tables 2 to 10 The presence of HT associated biomarkers is indicative of a susceptibility to hypertension. The kits provided for diagnosing a susceptibility to hypertension in an individual comprise wholly or in part protocol and reagents for detecting one or more biomarkers and interpretation software for data analysis and risk assessment.

In one typical embodiment, the HT risk biomarker information obtained using the methods and test kits of this invention are combined with other information concerning the individual, e.g. results from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of HT and diabetes. Clinical information collected by examination includes e.g. information concerning height, weight, hip and waist circumference, systolic and diastolic BP, and heart rate.

In one embodiment, the methods and kits of the invention are used in early detection of HT at or before disease onset, thus reducing or minimizing the debilitating effects of HT. In a preferred embodiment the methods and kits are applied in individuals who are free of clinical symptoms and signs of HT, but have family history of HT or in those who have multiple risk factors of HT.

In a second aspect, the present invention provides methods and kits for molecular diagnosis i.e. determining a molecular subtype of HT in an individual. In one preferred embodiment, molecular subtype of HT in an individual is determined to provide information of the molecular etiology of HT. When the molecular etiology is known, better diagnosis and prognosis of HT can be made and efficient and safe therapy for treating HT in an individual can be selected on the basis of the HT subtype data. For example, the drug that is likely to be effective, i.e. blood pressure lowering, can be selected without trial and error. In other embodiment, biomarker information obtained from methods and kits for determining molecular subtype of HT in an individual is for monitoring the effectiveness of their treatment. In one embodiment, methods and kits for determining molecular subtype of HT are used to select human subjects for clinical trials testing antihypertensive drugs and other therapies. The kits provided for detecting a molecular subtype of HT in an individual comprise wholly or in part protocol and reagents for detecting one or more biomarkers and interpretation software for data analysis and HT molecular subtype assessment.

In a third aspect, the present invention relates to methods and kits for identifying agents that modulate metabolic activity of a HT risk gene set forth in table 1. Such screening methods and kits are useful when developing drugs and other therapies having effect on a HT risk gene of table 1, or on a related metabolic pathway thereof. The methods and kits comprise exposing cells expressing one or more HT and/or obesity risk genes disclosed in table 1 to a potential modulator and measuring the effect of the potential modulator on activity or function of one or more HT risk genes or their encoded polypeptides, or on related metabolic pathways. Useful measurements include, but are not limited to expression and mRNA structure of a HT risk gene, concentration, structure, substrate specificity and biological activity of a HT risk gene encoded polypeptide, degradation rate of a HT risk gene encoded polypeptide or mRNA, and biological activity of a HT risk gene related metabolic pathway. Potential modulators include, but are not limited to, binding partners, agonists, antagonists and antibodies of a HT risk gene encoded polypeptides.

In a fourth aspect, the present invention relates to novel therapies, pharmaceutical or dietary compositions and kits for preventing and/or treating HT in an individual comprising administering, in a pharmaceutical or dietary composition, an agent, a recombinant protein or a nucleic acid modulating metabolic activity of a HT risk gene set forth in table 1. In a preferred embodiment, these compositions, methods or kits are used in an individual having HT or a susceptibility to HT to compensate altered expression of a HT risk gene, altered biological activity of HT risk gene encoded polypeptides or altered function of a HT risk gene related metabolic pathway when compared to healthy individuals of the same species.

### DETAILED DESCRIPTION OF THE INVENTION

The present invention relates to previously unknown associations between essential hypertension and various genes, loci and polymorphisms. These HT associated genes, loci and polymorphisms provide basis for novel methods and kits for risk assessment, diagnosis and prognosis of HT. In addition these genes, loci and markers provide basis for methods and kits for novel therapies to prevent, treat and/or reduce risk of HT in an individual.

A “biomarker” in the context of the present invention refers to a SNP marker disclosed in tables 2 to 10 or to a polymorphism of a gene disclosed in table 1 or at a locus closely linked thereto, or to an organic biomolecule which is related to a gene set forth in table 1 and which is differentially present in samples taken from subjects (patients) having HT compared to comparable samples taken from subjects who do not have HT. An “organic biomolecule” refers to an organic molecule of biological origin, e.g., steroids, amino acids, nucleotides, sugars, polypeptides, polynucleotides, complex carbohydrates or lipids. A biomarker is differentially present between two samples if the amount, structure, function or biological activity of the biomarker in one sample differs in a statistically significant way from the amount, structure, function or biological activity of the biomarker in the other sample.

A “haplotype,” as described herein, refers to any combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. E.g. the haplotype AGG defined by the SNP markers rs2202564, rs9564765 and rs803815 of this invention is the same as haplotype rs2202564, rs9564765 and rs803815 (TCC) in which the alleles are determined from the other strand, or haplotype rs2202564, rs9564765 and rs803815 (TGG), in which the first allele is determined from the other strand. The haplotypes described herein are differentially present in individuals with HT than in individuals without HT. Therefore, these haplotypes have diagnostic value for risk assessment, diagnosis and prognosis of HT in an individual. Detection of haplotypes can be accomplished by methods known in the art used for detecting nucleotides at polymorphic sites. The haplotypes described herein, e.g. having markers such as those shown in tables 4 and 10 are found more frequently in individuals with HT than in individuals without HT. Therefore, these haplotypes have predictive value for detecting HT or a susceptibility to HT in an individual. Some of the haplotypes shown in tables 4 and 10 are found less frequently in individuals with HT than in individuals without HT thus reducing the risk of HT.

A nucleotide position in genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene e.g. in NCBI databases (www.ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a HT susceptibility gene. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a HT susceptibility gene.

The SNP markers to which we have disclosed novel HT associations in tables 2 to 10 of this invention have been known in prior art with their official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP. For example the SNP having rs ID rs2202564 is SNP is in chromosome 13, variable alleles are A and G, and the nucleotide sequence assigned to rs2202564 is (R denotes the variable base; Genomic build 127) (SEQ ID NO: 1):

ACATATAGGT CAATCTGAAA AGGTGGAAGA GAAGTGAAAA
GCAATTCTTG TGCTCTAGTC AGTAGTGTGT TTATCTTTGA
CAGCCATTAC GTGTCAAAAA TTACTGACCC TTACTTAATG
ATATCTCTAT TGTTTTGGGA AGCCTAAGCA GTGGTAATAA
ATAGGCCCAA TAGGTATCAT GAATCCTACA TCATCGATGA
TCATTCTTGC TTGCTTCACC ACACAGGCAC GTGTTCCCAA
TTTGCAGCAA TTCTTTGCAG CTATTCCGGT GTCCATGCTT
CTTGCTTTTT GTAACCCTAC TATTTCTATA ATCCCTATAA
TCTGCACTCA TTCATAGGGG AGGAAAGAAG ACACAGACGG
GGCAAGGCCA CTTTTTGAAC GCCTCAGCCT AGAAATGCGC
TATGCCACTC ATTCTCACAT TCTTTCTTCT AGAAATGGCC
ACACCTAACA GCAAGGGAGG AAGGAACACA TAGTCTGGTA
TGTCCAGGAT GAAGAGAACA TAAATTTAAA TAAACAGTTT
GCAGTCTCCA TCACATTATT CR GAGATTAAAA ATATTTTTCT
CAAGTAAAGA TCTTTCTTAG AGATTAGCTT TGAAAATAAA
GATGGTACAA TATCCTAAAT TTATTTGCTG CAAGATAATT
TTACAATGTG GCCACATCTG ATCAGGCTTA ATAACCA

Although the numerical chromosomal position of a SNP may still change upon annotating the current human genome build the SNP identification information such as variable alleles and flanking nucleotide sequences assigned to a SNP will remain the same. Those skilled in the art will readily recognize that the analysis of the nucleotides present in one or more SNPs set forth in tables 2 to 10 of this invention in an individual's nucleic acid can be done by any method or technique capable of determining nucleotides present in a polymorphic site using the sequence information assigned in prior art to the rs IDs of the SNPs listed in tables 2 to 10 of this invention As it is obvious in the art the nucleotides present in polymorphisms can be determined from either nucleic acid strand or from both strands.

It is understood that the HT associated SNP markers and haplotypes described in tables 2 to 10 of this invention may be associated with other polymorphisms present in same HT associated genes and loci of this invention. This is because the SNP markers listed in tables 2 to 10 are so called tagging SNPs (tagSNPs). TagSNPs are loci that can serve as proxies for many other SNPs. The use of tagSNPs greatly improves the power of association studies as only a subset of loci needs to be genotyped while maintaining the same information and power as if one had genotyped a larger number of SNPs. These other polymorphic sites associated with the SNP markers listed in tables 2 to 10 of this invention may be either equally useful as biomarkers or even more useful as causative variations explaining the observed HT association of SNP markers and haplotypes of this invention.

The term “gene,” as used herein, refers to an entirety containing entire transcribed region and all regulatory regions of a gene. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA. Each gene of the HT associated genes disclosed in table 1 of this invention has been assigned a specific and unique nucleotide sequence by the scientific community. By using the name of a HT associated gene provided in table 1 those skilled in the art will readily find the nucleotide sequences of a gene and it's encoded mRNAs as well as amino acid sequences of it's encoded polypeptides although some genes may have been known with other name(s) in the art.

In certain methods described herein, an individual who is at risk for hypertension is an individual in whom one or more HT associated polymorphisms selected from the tables 2 to 10 of this invention are identified. In other embodiment also polymorphisms associated to SNPs and haplotypes of the tables 2 to 10 may be used in risk assessment of HT. The significance associated with an allele or a haplotype is measured by an odds ratio. In a further embodiment, the significance is measured by a percentage. In one embodiment, a significant risk is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors such as family history of HT, central or other type of obesity, lack of physical activity, high sodium intake, high alcohol intake, high intake of saturated fats, low intake of potassium and/or magnesium, low HDL cholesterol, diabetes mellitus, glucose intolerance, insulin resistance, the metabolic syndrome, and inflammation.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids (Nielsen P E et al, 1991). Probes or primers typically comprise about 15, to 30 consecutive nucleotides present e.g. in human genome and they may further comprise a detectable label, e.g., radioisotope, fluorescent compound, enzyme, or enzyme co-factor. Probes and primers to a SNP marker disclosed in tables 2 to 10 are available in the art or can easily be designed using the flanking nucleotide sequences assigned to a SNP rs ID and standard probe and primer design tools. Primers and probes (publicly available or designed) for SNP markers disclosed in tables 2 to 10 can be used in risk assessment as well as molecular diagnostic methods and kits of this invention.

The invention comprises polyclonal and monoclonal antibodies that bind to a polypeptide encoded by a HT associated gene set forth in table 1 of the invention. The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′).sub.2 fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

An antibody specific for a polypeptide encoded by a HT associated gene set forth in table 1 of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to HT or as part of a clinical testing procedure, e.g., to, for example, determine the efficacy of a given treatment regimen. Highly purified antibodies (e.g. monoclonal humanized antibodies specific to a polypeptide encoded by a HT associated gene of this invention) may be produced using GMP-compliant manufacturing processes known in the art. These “pharmaceutical grade” antibodies can be used in novel therapies modulating activity and/or function of a polypeptide encoded by a HT associated gene disclosed in table 1 of this invention to treat HT.

“A HT related condition” in the context of this invention refers to cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

### In Vitro Methods and Test Kits

The HT associated biomarkers of this invention provide novel in vitro methods and test kits, which can be used when making risk assessment, molecular diagnosis or prognosis of HT or a HT related condition for an individual. The disclosed methods and test kits do not require interaction with the body of a subject during the biomarker detection, instead only a test sample containing the biomarkers and representing the subject is needed. In practice to make risk assessment, molecular diagnosis or prognosis of HT or a HT related condition for an individual the methods and test kits are used in vitro e.g. in a clinical laboratory (i) to determine the presence of one or more HT associated biomarkers of this invention in a biological sample representing said individual and (ii) to compare the biomarker data of the subject to the biomarker data of healthy and hypertensive. The biomarker data of a subject obtained using the in vitro methods and test kits of this invention may be combined with non-genetic data of the subject to make risk assessment, molecular diagnosis or prognosis of HT or a HT related condition.

The methods and test kits provided for risk assessment, molecular diagnosis or prognosis of HT or a HT related condition of an individual comprise wholly or in part protocol and reagents for detecting one or more HT associated biomarkers and interpretation software for data analysis and risk assessment. Prior using the disclosed methods and test kits of this invention a biological sample is needed from a subject to be tested. Any biological sample representing the subject and containing the biomarkers, which are to be detected from the subject can be used. Typically a biological sample is taken by a health care professional e.g. by a MD or by a nurse and it comprises blood, saliva, buccal cells or urine. In some cases a subject may collect a biological sample (e.g. a saliva sample) himself or herself. To minimize degradation of the HT associated biomarkers during the sample collection, storage and transportation a biological sample may be collected to a tube or to a vial containing stabilizers and chemicals inactivating interfering agents from the collected sample. Prior to biomarker analyses in a test laboratory biological samples to be tested typically need processing, e.g. if the biomarkers are SNP-markers processing may comprise genomic DNA extraction and DNA quality (integrity) assessment.

One major application of the current invention is detecting a susceptibility to HT or a HT related condition. The risk assessment methods and test kits of this invention can be applied to any healthy person as a screening or predisposition test, although the methods and test kits are preferably applied to high-risk individuals (who have e.g. family history of HT, central or other type of obesity, lack of physical activity, high sodium intake, high alcohol intake, high intake of saturated fats, low intake of potassium and/or magnesium, low HDL cholesterol, diabetes mellitus, glucose intolerance, insulin resistance and the metabolic syndrome, elevated inflammatory marker, or any combination of these or an elevated level of any other risk factor for HT). Molecular tests that define genetic factors contributing to HT might be used together with or independent of the known clinical risk factors to define an individual's risk relative to the general population. Better means for identifying those individuals susceptible for HT should lead to better preventive and treatment regimens, including more aggressive management of the risk factors for HT such as central or other type of obesity, lack of physical activity, high sodium intake, high alcohol intake, high intake of saturated fats, low intake of potassium and/or magnesium, low HDL cholesterol, elevated blood glucose, glucose intolerance, insulin resistance, the metabolic syndrome and inflammatory components as reflected by increased C-reactive protein levels or other inflammatory markers. Physicians may use the information on genetic risk factors to convince particular patients to adjust their life style e.g. to stop smoking, to change their diet or to increase exercise. A detected high risk of HT may also motivate the HT patients to improved compliance to antihypertensive treatments such as drugs and functional food products. The latter include antihypertensive peptides.

In one embodiment of the invention, detection of a susceptibility to HT in a subject, is made by determining one or more SNP markers and haplotypes disclosed in tables 2 to 10 of this invention in the subject's nucleic acid. The presence of HT associated alleles of the assessed SNP markers and haplotypes in individual's genome indicates subject's increased risk for HT. The invention also pertains to methods of diagnosing a susceptibility to HT in an individual comprising detection of a haplotype in a HT risk gene that is more frequently present in an individual having HT (affected), compared to the frequency of its presence in a healthy individual (control), wherein the presence of the haplotype is indicative of a susceptibility to HT. A haplotype may be associated with a reduced rather than increased risk of HT, wherein the presence of the haplotype is indicative of a reduced risk of HT. In other embodiment of the invention, diagnosis of susceptibility to HT, is done by detecting in the subject's nucleic acid one or more polymorphic sites which are in linkage disequilibrium with one or more SNP markers and haplotypes disclosed in tables 2 to 10 of this invention. The most useful polymorphic sites for in vitro methods and test kits are those altering the biological activity of a polypeptide encoded by a HT associated gene set forth in table 1. Examples of such functional polymorphisms include, but are not limited to frame shifts, premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other useful polymorphic sites are those affecting transcription of a HT associated gene set forth in table 1, or translation of it's mRNA due to altered tissue specificity, due to altered transcription rate, due to altered response to physiological status, due to altered translation efficiency of the mRNA and/or due to altered stability of the mRNA. The presence of nucleotide sequence variants altering the polypeptide structure and/or expression in HT associated genes of this invention in individual's nucleic acid is indicative for susceptibility to HT.

In biomarker assays determination of the nucleotides present in one or more HT associated SNP markers of this invention, as well as polymorphic sites associated with HT associated SNP markers of this invention, in an individual's nucleic acid can be done by any method or technique which can accurately determine nucleotides present in a polymorphic site. Numerous suitable methods have been described in the art (see e.g. Kwok P-Y, 2001; Syvänen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, a susceptibility to HT is assessed from transcription products of one or more HT associated genes. Qualitative or quantitative alterations in transcription products can be assessed by a variety of methods described in the art, including e.g. hybridization methods, enzymatic cleavage assays, RT-PCR assays and microarrays. A test sample from an individual is collected and the alterations in the transcription of HT associated genes are assessed from the RNA molecules present in the sample. Altered transcription is diagnostic for a susceptibility to HT.

In another embodiment of the invention, detection of a susceptibility to HT is made by examining expression, abundance, biological activities, structures and/or functions of polypeptides encoded by one or more HT related genes disclosed in table 1. A test sample from an individual is assessed for the presence of alterations in the expression, biological activities, structures and/or functions of the polypeptides, or for the presence of a particular polypeptide variant (e.g., an isoform) encoded by a HT risk gene. An alteration can be, for example, quantitative (an alteration in the quantity of the expressed polypeptide, i.e., the amount of polypeptide produced) or qualitative (an alteration in the structure and/or function of a polypeptide encoded by a HT risk gene, i.e. expression of a mutant polypeptide or of a different splicing variant or isoform). Alterations in expression, abundance, biological activity, structure and/or function of a HT susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the HT susceptibility polypeptide itself or it's fragment or from substrates and reaction products of said polypeptide. A control sample is a sample that corresponds to the test sample (e.g., is from the same type of cells), and is from an individual who is not affected by HT. An alteration in the expression, abundance, biological activity, function or composition of a polypeptide encoded by a HT susceptibility gene of the invention in the test sample, as compared with the control sample, is indicative of a susceptibility to HT. In another embodiment, assessment of the splicing variant or isoform(s) of a polypeptide encoded by a polymorphic or mutant HT risk gene can be performed directly (e.g., by examining the polypeptide itself, or indirectly (e.g., by examining the mRNA encoding the polypeptide, such as through mRNA profiling).

Yet in another embodiment, a susceptibility to HT can be detected by assessing the status and/or function of biological networks and/or metabolic pathways related to one or more polypeptides encoded by HT risk genes of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop HT is evaluated by comparing observed status and/or function of biological networks and or metabolic pathways of a subject to the status and/or function of biological networks and or metabolic pathways of healthy controls.

Another major application of the current invention is determination of a molecular subtype of HT in a subject. In vitro methods and kits of this invention can be applied to a person having HT, although the methods and test kits are preferably applied to persons having familial essential hypertension (who have family members with HT). In one preferred embodiment, molecular subtype of HT in an individual is determined to provide information of the molecular etiology of HT. When the molecular etiology is known, better diagnosis and prognosis of HT can be made and efficient and safe therapy for treating HT in an individual can be selected on the basis of this HT subtype. For example, the drug that is likely to be effective, i.e. blood pressure lowering, can be selected without trial and error. Physicians may use the information on genetic risk factors with or without known clinical risk factors to convince particular patients to adjust their life style and manage HT risk factors and select intensified preventive and curative interventions for them. In other embodiment, biomarker information obtained from methods and kits for determining molecular subtype of HT in an individual is for monitoring the effectiveness of their treatment. In one embodiment, methods and kits for determining molecular subtype of HT are used to select human subjects for clinical trials testing antihypertensive drugs or other therapies. The kits provided for determination of a molecular subtype of HT in an individual comprise wholly or in part protocol and reagents for detecting one or more biomarkers and interpretation software for data analysis and HT molecular subtype assessment.

The methods and test kits of the invention may further comprise a step of combining non-genetic information with the biomarker data to make risk assessment, molecular diagnosis or prognosis of HT or a HT related condition. Useful non-genetic information comprises age, gender, ethnicity, the family history of HT, CVD, obesity, diabetes and hypercholesterolemia, and the medical history concerning CVD, obesity, diabetes and hypercholesterolemia of the subject. The detection method of the invention may also further comprise a step determining blood, serum or plasma cholesterol, HDL cholesterol, LDL cholesterol, triglyceride, apolipoprotein B and AI, fibrinogen, ferritin, transferrin receptor, C-reactive protein, serum or plasma insulin concentration, vasoactive peptides and dietary intake of relevant nutrients such as sodium, other minerals such as potassium, magnesium, calcium, selenium, and alcohol, saturated and unsaturated fatty acids, amino acids, and dietary antioxidants such as vitamin C and E.

The score that predicts the probability of HT may be calculated e.g. using a multivariate failure time model or a logistic regression equation. The results from the further steps of the method as described above render possible a step of calculating the probability of HT using a logistic regression equation as follows. Probability of HT=1/[1+e (−(−a+Σ(bi*Xi))], where e is Napier's constant, Xi are variables related to the HT, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bi are between −20 and 20; and for i between 0 (none) and 100,000. A negative coefficient bi implies that the marker is risk-reducing and a positive that the marker is risk-increasing. Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of HT as percentage of risk in one year, two years, five years, 10 years or 20 years. Alternative statistical models are failure-time models such as the Cox's proportional hazards' model, other iterative models and neural networking models.

In vitro test kits (e.g. reagent kits) of this invention comprise reagents, materials and protocols for assessing one or more biomarkers, and instructions and software for comparing the biomarker data from a subject to biomarker data from healthy and diseased people to make risk assessment, diagnosis or prognosis of HT. Useful reagents and materials for kits include, but are not limited to PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, antibodies which bind to altered or to non-altered (native) HT risk gene encoded polypeptide, means for amplification of nucleic acids fragments from one or more HT risk genes selected from the table 1, means for analyzing the nucleic acid sequence of one or more HT risk genes or fragments thereof, or means for analyzing the sequence of one or more amino acid residues of HT risk gene encoded polypeptides, etc. In one embodiment, a kit for diagnosing susceptibility to HT comprises primers and reagents for detecting the nucleotides present in one or more SNP markers selected from the tables 2 to 10 in individual's nucleic acid.

Yet another application of the current invention is related to methods and test kits for monitoring the effectiveness of a treatment for HT. The disclosed methods and kits comprise taking a tissue sample (e.g. peripheral blood sample or adipose tissue biopsy) from a subject before starting a treatment, taking one or more comparable samples from the same tissue of the subject during the therapy, assessing expression (e.g., relative or absolute expression) of one or more HT risk genes set forth in table 1 in the collected samples of the subject and detecting differences in expression related to the treatment. Differences in expression can be assessed from mRNAs and/or polypeptides encoded by one or more HT risk genes of the invention and an alteration in the expression towards the expression observed in the same tissue in healthy individuals indicates the treatment is efficient. In a preferred embodiment the differences in expression related to a treatment are detected by assessing biological activities of one or more polypeptides encoded by HT risk genes set forth in table 1.

Alternatively the effectiveness of a treatment for HT can be followed by assessing the status and/or function of metabolic pathways related to one or more polypeptides encoded by HT risk genes set forth in table 1. Status and/or function of a metabolic pathway can be assessed e.g. by measuring amount or composition of one or morel polypeptides, belonging to the metabolic pathway, from a biological sample taken from a subject before and during a treatment. Alternatively status and/or function of a metabolic pathway can be assessed by measuring one or more metabolites belonging to the metabolic pathway, from a biological sample before and during a treatment. Effectiveness of a treatment is evaluated by comparing observed changes in status and/or function of metabolic pathways following treatment with HT therapeutic agents to the data available from healthy subjects.

### Methods of Therapy

The present invention discloses novel methods for the prevention and treatment of HT. In particular, the invention relates to methods of treatment for HT or susceptibility to HT as well as to methods of treatment for manifestations and subtypes of HT.

The term “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition.

The present invention encompasses methods of treatment (prophylactic and/or therapeutic) for HT using a HT therapeutic agent. A “HT therapeutic agent” is an agent that alters (e.g., enhances or inhibits) enzymatic activity or function of a HT risk affecting polypeptide, and/or expression of a HT risk gene disclosed in table 1. Useful therapeutic agents can alter biological activity or function of a HT susceptibility polypeptide and/or expression of related gene by a variety of means, for example, by altering translation rate of a HT susceptibility polypeptide encoding mRNA; by altering transcription rate of a HT risk gene; by altering posttranslational processing rate of a HT susceptibility polypeptide; by interfering with a HT susceptibility polypeptide biological activity and/or function (e.g., by binding to a HT susceptibility polypeptide); by altering stability of a HT susceptibility polypeptide; by altering the transcription rate of splice variants of a HT risk gene or by inhibiting or enhancing the elimination of a HT susceptibility polypeptide from target cells, organs and/or tissues.

Representative therapeutic agents of the invention comprise the following: (a) nucleic acids, fragments, variants or derivatives of the HT associated genes disclosed in table 1 of this invention, nucleic acids encoding a HT susceptibility polypeptide or an active fragment or a derivative thereof and nucleic acids modifying the expression of said HT associated genes (e.g. antisense polynucleotides, catalytically active polynucleotides (e.g. ribozymes and DNAzymes), molecules inducing RNA interference (RNAi) and micro RNA), and vectors comprising said nucleic acids; (b) HT susceptibility polypeptides encoded by genes set forth in table 1, active fragments, variants or derivatives thereof, binding agents of HT susceptibility polypeptides; peptidomimetics; fusion proteins or prodrugs thereof, antibodies (e.g., an antibody to a mutant HT susceptibility polypeptide, or an antibody to a non-mutant HT susceptibility polypeptide, or an antibody to a particular variant encoded by a HT risk gene, as described above) and other polypeptides (e.g., HT susceptibility polypeptide receptors, active fragments, variants or derivatives thereof); (c) metabolites of HT susceptibility polypeptides or derivatives thereof; (d) small molecules and compounds that alter (e.g., inhibit or antagonize) a HT risk gene expression, activity and/or function of a HT risk gene encoded polypeptide, or activity and/or function of a HT gene related metabolic pathway and; (e) small molecules and compounds that alter (e.g. induce, agonize or modulate) a HT risk gene expression, activity and/or function of a HT risk gene encoded polypeptide, or activity and/or function of a HT gene related metabolic pathway.

The nucleic acid sequences assigned in the art to the HT associated genes provided in table 1 of this invention are publicly available and can be used to design and develop therapeutic nucleic acid molecules and recombinant DNA molecules for the prevention and treatment of HT. For example antisense nucleic acid molecules targeted to a gene listed in table 1 can be designed using tools and the nucleotide sequence of the gene available in the art and constructed using chemical synthesis and/or enzymatic ligation reactions using procedures known in the art. For example, an antisense nucleic acid molecule (e.g., an antisense oligonucleotide) can be chemically synthesized using naturally occurring nucleotides or modified nucleotides designed to increase the biological stability of the molecules or to increase the physical stability of the duplex formed between the antisense oligonucleotide and sense nucleic acids, e.g., phosphorothioate derivatives and acridine substituted nucleotides can be used. Alternatively, the antisense nucleic acid molecule can be produced biologically using an expression vector into which a nucleic acid molecule encoding a HT risk gene, a fragment or a variant thereof has been cloned in antisense orientation (i.e., RNA transcribed from the expression vector will be complementary to the transcribed RNA of a HT risk gene of interest).

More than one HT therapeutic agent can be used concurrently, if desired. The therapy is designed to alter (e.g., inhibit or enhance), replace or supplement activity and/or function of one or more HT polypeptides or related metabolic pathways in an individual. For example, a HT therapeutic agent can be administered in order to upregulate or increase the expression or availability of a HT risk gene encoded polypeptide or it's specific variant or, conversely, to downregulate or decrease the expression or availability of a HT risk gene encoded polypeptide or a specific variant thereof. Upregulation or increasing expression or availability of a native HT risk gene encoded polypeptide or it's particular variant in an individual could e.g. compensate for the low or altered biological activity of a defective gene or variant; whereas downregulation or decreasing expression or availability of a defective HT risk gene encoded polypeptide or it's particular splicing variant in an individual could minimize the impact of the defective gene or the particular variant.

The HT therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid encoding a HT susceptibility polypeptide, fragment, variant or derivative thereof, either by itself or included within a vector, can be introduced into cells of an individual affected by HT using variety of experimental methods described in the art, so that the treated cells start to produce native HT susceptibility polypeptide. Thus, cells which, in nature, lack of a native HT risk gene expression and activity, or have abnormal HT risk gene expression and activity, can be engineered to express a HT susceptibility polypeptide or an active fragment or a different variant of said HT susceptibility polypeptide. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle). Alternatively, in another embodiment of the invention, a nucleic acid (e.g. a polynucleotide) which specifically hybridizes to the mRNA and/or genomic DNA of a HT risk gene is administered in a pharmaceutical composition to the target cells or said nucleic acid is generated “in vivo”. The antisense nucleic acid that specifically hybridizes to the mRNA and/or DNA inhibits expression of the HT susceptibility polypeptide, e.g., by inhibiting translation and/or transcription. Binding of the antisense nucleic acid can be due to conventional base pairing, or, for example, in the case of binding to DNA duplexes, through specific interaction in the major groove of the double helix. In a preferred embodiment nucleic acid therapeutic agents of the invention are delivered into cells that express one or more HT risk genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a HT risk gene. Such vectors can be constructed by various recombinant DNA technology methods standard in the art.

The expression of a HT risk gene disclosed in table 1 may be reduced e.g. by inactivating or “knocking out” it or its promoter using targeted homologous recombination methods described in the art. Alternatively, expression of a functional, non-mutant HT risk gene can be increased using a similar method: targeted homologous recombination can be used to replace a non-functional HT risk gene with a functional form of the said gene in a cell. In yet another embodiment of the invention, other HT therapeutic agents as described herein can also be used in the treatment or prevention of HT. The therapeutic agents can be delivered in a pharmaceutical composition they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to meet pharmaceutical requirements using standard methods described in the art. A combination of any of the above methods of treatment (e.g., administration of non-mutant HT susceptibility polypeptide in conjunction with RNA molecules inducing RNA interference targeted to the mutant HT susceptibility mRNA) can also be used.

In the case of pharmaceutical therapy, the invention comprises compounds, which enhance or reduce the activity and/or function of at least one polypeptide encoded by HT susceptibility genes set forth in table 1. The treatment may also enhance or reduce the expression of one or more genes selected from HT susceptibility genes set forth in table 1. In another embodiment of the invention, pharmaceutical therapy of the invention comprises compounds, which enhance or reduce the activity and/or function of one or more metabolic pathways related to HT susceptibility genes, proteins or polypeptides. The treatment may also enhance or reduce the expression of one or more genes in metabolic pathways related to HT susceptibility genes, proteins or polypeptides.

Furthermore, a disclosed method or a test based on HT susceptibility gene specific biomarkers (e.g. polymorphic sites, expression or polypeptides) is useful in selecting drug therapy for patients with HT. For example when the less frequent, i.e. the minor, assumable mutated allele in the HT susceptibility gene is risk-reducing, and if said mutation is a gene function reducing mutation, one can deduce that the gene function and/or activity would increase the risk of HT. On that basis, drugs and other therapies such as gene therapies that reduce or inhibit the function or activity of the HT susceptibility gene or the encoded protein would reduce the risk of the said disease and could be used to both prevent and treat the said disease in subjects having said mutated allele.

In another embodiment of the invention a HT therapeutic agent comprises a know therapeutic agent related to a HT associated gene listed in table 1 of this invention but which is not used to treat HT. Such agents are useful for developing new therapies for HT as they probably are agonizing, modulating, binding, inhibiting and/or antagonizing (i) expression of a HT risk gene, (ii) biological activity and/or function of a HT risk gene encoded polypeptide, or (iii) biological activity and/or function of a HT risk gene related metabolic pathway. These agents may be used alone or with combination with other treatments and agents used for prevention or treatment of HT.

### Pharmaceutical Compositions

The present invention also pertains to pharmaceutical compositions comprising agents described herein, particularly polynucleotides, polypeptides and any fractions, variants or derivatives of HT susceptibility genes, and/or agents that alter (e.g., enhance or inhibit) expression of a HT risk gene or genes, or activity of one or more polypeptides encoded by HT susceptibility genes as described herein. For instance, an agent that alters expression of a HT risk gene, or activity of one or more polypeptides encoded by HT susceptibility genes or a HT susceptibility polypeptide binding agent, binding partner, fragment, fusion protein or prodrug thereof, or polynucleotides of the present invention, can be formulated with a physiologically acceptable carrier or excipient to prepare a pharmaceutical composition. The carrier and composition can be sterile. The formulation should suit the mode of administration. In a preferred embodiment pharmaceutical compositions comprise agent or agents reversing, at least partially, HT associated changes in metabolic pathways related to the HT associated genes disclosed in table 1 of this invention.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents. The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents. The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. For topical application, non-sprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of HT, and should be decided according to the judgment of a practitioner and each patient's circumstances. Effective doses may be extrapolated from dose-response curves derived from in vitro or animal model test systems.

### Functional Foods

By definition “functional foods” or “nutraceuticals” are foods or dietary components or food ingredients that may provide a health benefit beyond basic nutrition. Functional foods are regulated by authorities (e.g. by the FDA in US) according to their intended use and the nature of claims made on the package. Functional foods can be produced by various methods and processes known in the art including, but not limited to synthesis (chemical or microbial), extraction from a biological material, mixing functional ingredient or component to a regular food product, fermentation or using a biotechnological process. A functional food may exert its effects directly in the human body or it may function e.g. through human intestinal bacterial flora.

The polypeptides encoded by the HT associated genes disclosed in table 1 of this invention can be used as molecular targets towards which functional foods claiming health benefit in HT can be developed. In one embodiment a functional food may be developed to compensate altered biological activity of a polypeptide encoded by a HT risk gene set forth in table 1 or a related metabolic pathway. For example if the reduced biological activity of a HT risk gene encoded polypeptide or a related metabolic pathway is associated with increased risk of hypertension a functional food may be developed to activate or stabilize the HT risk gene encoded polypeptide, or to contain a metabolite which is normally produced by the HT risk gene encoded polypeptide. Similarly, if the increased biological activity of a HT risk gene encoded polypeptide or a related metabolic pathway is associated with increased risk of hypertension a functional food may be developed either to inhibit the expression of the HT risk gene or to inhibit the biological activity of the HT risk gene encoded polypeptide or a related metabolic pathway.

### EXPERIMENTAL SECTION

### Example 1

### Hypertension Study in Eastern Finnish, Ashkenazi Jewish, German and English Subjects: the Study Subjects and Genome Wide Scanning Using Illumina's HumanHap300

The subjects for this hypertension whole genome association study were selected from the 500 T2D cases and the 497 T2D-free controls of the Jurilab's whole genome association study in type 2 diabetes (DiaGen study) covered by the U.S. patent application Ser. No. 60/863,438. The 586 hypertension study subjects included 114 hypertensive cases and 114 controls from Eastern Finland, 110 hypertensive cases and 110 controls from Israel (Ashkenazi Jewish), 41 hypertensive cases and 41 controls from Germany and 28 hypertensive cases and 28 controls from England.

### Definition of Cases and Controls

The current work was based on 293 hypertensive cases and 293 normotensive controls, a total of 586 subjects. The cases had either previous diagnosis of HT or medication for hypertension. The controls had neither diagnosis of HT nor antihypertensive medication.

Both the cases and controls had the following:
1. A written informed consent which will allow us to use data and samples for the commercial applications,
2. Extracted DNA or whole blood for DNA extraction, plasma and serum,
3. Information (data) on age, gender and ethnicity (for matching) and
4. Information about the family history of diseases defined in the questionnaire.

From each of the four populations (Eastern Finns, Ashkenazi Jews, Germans and English), an equal number of cases and controls were selected and matched for gender.

### Eastern Finnish (EF) Study Subjects

The current population of the North Savo is over 250,000 people. The population is genetically homogenous and has a high prevalence of type 2 diabetes. Mailed health-related surveys show consistently very high participation rates. There is almost no illiteracy. The “North Savo Health Survey” was approved by the local ethics committee and it was carried out in October to December, 2003. The survey was targeted to all households in the municipalities of Kuopio, Karttula, Lapinlahti, Leppävirta, Maaninka, Rautalampi, Siilinjäri, Suonenjoki, Tervo, Vehmersalmi, and Vesanto. The number of households was about 70,000 and the number of people over 18 years old was about 200,000. A letter was sent to each household containing three personal and one common questionnaire. The three oldest persons who were at least 18 years of age in the household were asked to fill in the personal questionnaire and one of them to fill in the common family data questionnaire, and return them in the same single return envelope. Only persons, who gave the consent to obtain their hospital records and who provided their personal identification code, were asked to return the questionnaire. The “North Savo Project” included the collection of disease, family, drug response and contact information. By the end of 2004, 17,100 participants were surveyed. The North Savo Survey data were used to identify probands with hypertension.

The study subjects were participants in the “SOHFA” study. The “SOHFA” (Study of Diabetic, Obese and Hypertensive Families in the Northern Savo Genetic Epidemiology Cohort Study) is a contractual study, in which the University of Kuopio is the contractee.

Both systolic and diastolic BPs were measured in the morning by a nurse with a mercury sphygmomanometer. The measuring protocol included three measurements in standing position with 5-minute intervals. The mean of all three measurements were used as SBP and DBP. Body mass index (BMI) was computed as the ratio of weight to the square of height (kg/m²). Waist-to-hip ratio (WHR) was calculated as the ratio of waist circumference (average of one measure taken after inspiration and one taken after expiration at the midpoint between the lowest rib and the iliac crest) to hip circumference (measured at the level of the trochanter major). Age and tobacco smoking were recorded on a self-administered questionnaire checked by an interviewer.

### Ashkenazi Jewish (AJ) DiaGen Study Subjects

Subjects included in the study were collected in Israel by the physicians in charge in specialized clinics. Subjects were diagnosed with type 2 Diabetes Mellitus according to the etiologic classification of Diabetes Mellitus proposed by the International Expert Committee under the sponsorship of the American Diabetes Association on May 1997, We included in the study 200 subjects (82 males and 118 females, mean age 64), each with 3 or more blood relatives of second degree or closer, suffering from T2D.

Matching 200 healthy control subjects (82 males and 118 females, mean age 74) were collected from the Israeli blood bank and elderly patients visiting general practitioners clinics. All subjects were of Ashkenazi Jewish origin. The study was approved by the appropriate ethics committees and participants had signed informed consent forms. The 400 AJ DiaGen study subjects included 110 HT cases and 110 normotensive controls.

### German (GE) and English (UK) DiaGen Study Subjects

In Germany, cases were sampled from T2D patients from the Hospital of Diabetes and Metabolic Diseases (Karlsburg, Germany) and the diabetes dispensary unit of the Department of Endocrinology of the Ernst-Moritz-Arndt University (Greifswald, Germany). The controls were sampled from the non-diabetic examinees of the population based SHIP study cohort (Luedemann et al 2002). Total of 49 cases (24 females and 25 males) and 50 matched healthy controls (24 females and 26 males) from Germany were included in the DiaGen study. The 99 GE DiaGen study subjects included 41 HT cases and 41 normotensive controls.

From England total of 50 cases (31 females and 19 males) and 50 matched healthy controls (31 females and 19 males) were included in the DiaGen study. The controls were selected from the examinees of the Age and Cognitive Performance Research Centres (ACPRC) volunteer panel, a group of over 6000 older adults who have been previously described in detail (Rabbitt et al, 2004). A cohort of approximately 2000 of these individuals has DNA archived in the Dyne-Steel DNA bank. A group of 456 of these volunteers, residents of Greater Manchester, had previously taken part in a research study in 2001 which included medical history, including that of Diabetes Mellitus, and measurement of HbA_{1C}. From the original cohort of 456, a sample of 50 individuals was identified to sex match diabetic cases from Manchester. Each individual had an HbA_{1C} below 5.5% and at telephone interview of family diabetes mellitus history in 2006, reported no evidence of diabetes mellitus in parents or siblings. The University of Manchester research ethics committee approved the study and each individual completed an individual form of consent. The 100 UKi DiaGen study subjects included 28 HT cases and 28 HT normotensive controls.

### Genomic DNA Isolation and Quality Testing

High molecular weight genomic DNA from EF samples was extracted from frozen venous whole blood using standard methods (proteinase K digestion, phenol-chloroform extractions and precipitation) and dissolved in standard TE buffer. The quantity and purity of each DNA sample was determined by absorbance measurements done with NanoDrop ND-1000 Spectrophotometer (NanoDrop Technologies, Wilmington, Del. USA). A sample was qualified for genome wide scan (GWS) analysis if A260/A280 ratio was ≧1.7. Before GWS analysis the samples were diluted to concentration of 60 ng/μl in reduced EDTA TE buffer (TEKnova, Hollister, Calif., USA).

### Genome-Wide Scanning Using Illumina's HumanHap300

The whole-genome genotyping of the DNA samples was performed by using Illumina's Sentrix HumanHap300 BeadChips and Infinium II genotyping assay. The HumanHap300 BeadChip contained over 317,000 tagSNP markers derived from the International HapMap Project. TagSNPs are loci that can serve as proxies for many other SNPs. The use of tagSNPs greatly improves the power of association studies as only a subset of loci needs to be genotyped while maintaining the same information and power as if one had genotyped a larger number of SNPs.

The Infinium II genotyping with the HumanHap300 BeadChip assays was performed according to the “Single-Sample BeadChip Manual process” described in detail in “Infinium™ II Assay System Manual” provided by Illumina (San Diego, Calif., USA). Briefly, 750 ng of genomic DNA from a sample was subjected to whole-genome amplification. The amplified DNA was fragmented, precipitated and resuspended to hybridization buffer. The resuspended sample was heat denatured and then applied to one Sentrix HumanHap300 beadchip. After overnight hybridization mis- and non-hybridized DNA was washed away from the BeadChip and allele-specific single-base extension of the oligonucleotides on the BeadChip was performed in a Tecan GenePaint rack, using labeled deoxynucleotides and the captured DNA as a template. After staining of the extended DNA, the BeadChips were washed and scanned with the BeadArray Reader (Illumina) and genotypes from samples were called by using the BeadStudio software (Illumina).

Infinium II genotyping with the HumanHap300 BeadChips were done for 500 T2D cases and 497 T2D-free controls including the 586 hypertension study subjects.

### Example 2

### Statistical Analyses of the GWS Data of the Hypertension Study (Example 1.)

### Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples genotyped successfully. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) cannot be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p2, 2pq, and q2 for genotypes ‘AA’, ‘AB’ and ‘BB’, respectively. If the SNP is not in H-W equilibrium it can be due to genotyping error or some unknown population dynamics (e.g. random drift, selection).

Following criteria were used in the statistical analysis: CR>90%, MAF>1%, and H-W equilibrium Chi-square test statistic <27.5 (the control group). A total of 315,917 Illumina300K SNPs fulfilled the above criteria.

### Single SNP Analysis

Differences in allele distributions between cases and controls were screened for all SNPs. The screening was carried out using the standard Chi-square independence test with 1 df (allele distribution, 2×2 table). SNPs that gave a P-value less tan 0.001 (Chi-square with 1 df of 10.23 or more) were considered statistically significant and reported in the tables. Odds ratio was calculated as ad/bc, where a is the number of minor alleles in cases, b is the number of major alleles in cases, c is the number of minor allele in controls, and d is the number of major alleles in controls. Minor allele was defined as the allele for a given SNP that had smaller frequency than the other allele in the control group.

### Genotype Analysis

Logistic regression (R-programming language) with three genetic models were tested: additive, recessive and dominance. As an example if the alleles of the SNP are A and C then additive model tests the linear increase in disease risk from genotype AA to AC to CC. In the dominance and recessive model heterozygous genotypes are combined with either AA or CC genotypes.

### Haplotype Analysis

The data set was analyzed with a haplotype pattern mining algorithm with HPM software (Toivonen H T et al, 2000). For HPM software, genotypes must be phase known to determine which alleles come from the mother and which from the father. Without family data, phases must be estimated based on population data. We used the HaploRec program (Eronen L et al, 2004) to estimate the phases. For phase-known data HPM finds all haplotype patterns that are in concordance with the phase configuration. The length of the haplotype patterns can vary. As an example, if there are four SNPs and an individual has alleles A T for SNP1, C C for SNP2, C G for SNP3, and A C for SNP4, then HPM considers haplotype patterns that are in concordance with the estimated phase (done by HaploRec). If the estimated phase is ACGA (from the mother/father) and TCCC (from the father/mother) then HPM considers only two patterns (of length 4 SNPs): ACGA and TCCC. A SNP is scored based on the number of times it is included in a haplotype pattern that differs between cases and controls (a threshold Chi-square value can be selected by the user). Significance of the score values was tested based on permutation tests. Several parameters can be modified in the HPM program including the Chi-square threshold value (−x), the maximum haplotype pattern length (−l), the maximum number of wildcards that can be included in a haplotype pattern (−w), and the number of permutation tests in order to estimate the P-value (−p).

### Results of the GWS Study (Example 1.)

In Table 1. the genes associated with hypertension are listed. Table 2 gives the SNP markers with the strongest association with HT in the individual marker analysis. The analysis is based on 140 HT cases and 182 healthy controls from East Finland. Below is the list of the tables where results of different statistical analysis are presented:

Table 3. Haplotype genomic regions with the strongest association with HT in the haplotype sharing analysis (HaploRec+HPM) with 8 SNPs. The analysis is based on 140 HT cases and 182 healthy controls from East Finland.

Table 4. Haplotypes with the strongest association with HT based on HaploRec+HPM analysis with 8 SNPs. The analysis is based on 140 HT cases and 182 healthy controls from East Finland.

Table 5. SNP markers with the strongest association with hypertension in the individual marker analysis. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

Table 6. SNP markers with the strongest association with hypertension in the regression analysis with an additive genotype model and T2D as a covariate. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

Table 7. SNP markers with the strongest association with hypertension in the regression analysis with a recessive genotype model and T2D as a covariate. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

Table 8. SNP markers with the strongest association with hypertension in the regression analysis with a dominant genotype model and T2D as a covariate. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

Table 9. Haplotype genomic regions with the strongest association with hypertension in the haplotype sharing analysis (HaploRec+HPM) with 5 SNPs. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

Table 10. Haplotypes with the strongest association with hypertension based on HaploRec+HPM analysis with 5 SNPs. The analysis is based on the combined data of 110 HT cases and 110 healthy controls from the Ashkenazi Jewish population, 114 HT cases and 114 healthy controls from the East Finnish population, 41 HT cases and 41 healthy controls from the German population and 28 HT cases and 28 healthy controls from the English population.

### Example 3

### Examples of the Content of the In Vitro Diagnostic Assays

The score that predicts the probability of HT may be calculated e.g. using a logistic regression equation: probability of HT=1/[1+e (−(−a+Σ(bi*Xi))], where e is Napier's constant, Xi are variables related to the HT, bi are coefficients of these variables in the logistic function, and a is the constant term in the logistic function, and wherein a and bi are preferably determined in the population in which the method is to be used, and Xi are preferably selected among the variables that have been measured in the population in which the method is to be used.

As an example the probability of HT may be estimated with the model Prob(HT)=1/[1+e(−(−a+b₁x₁+b₂x₂+b₃x₃+b₄x₄)], where bᵢ's are coefficients depending on the population and combination of xᵢ's and for each individual x₁-x₄ are any combination of the SNPs from the following list of SNPs: rs1721355, rs561264, rs2153184, rs9564765, rs8066575, rs6698312, rs2301301, rs7406978, rs2245192, and rs747250. The model may also include additional SNPs from the tables 2-10 or some of the xᵢ's may be other than SNPs including haplotypes, lifestyle and environmental factors.

### IMPLICATIONS AND CONCLUSIONS

We have discovered a total of 425 HT associated genes, in which any HT associated biomarkers can be used to predict HT, and thus these markers can be used to develop molecular diagnostic tests for HT or a HT related condition. In addition, we have disclosed a set of 1874 SNP markers predicting HT. The markers can also be used as part of pharmacogenetic tests used to predict the efficacy of a HT therapy and guide the selection of effective and safe treatment for a subject. The genes discovered are also useful in development of novel therapies such as drugs and dietary interventions for HT or a HT related condition. The genes and markers of this invention can also be used to screen, identify and test novel antihypertensive agents and compounds.

While this invention has been particularly shown and described with reference to preferred embodiments thereof, it will be understood by those skilled in the art that various changes in form and details may be made therein without departing from the spirit and scope of the invention as defined by the appended claims.

TABLE 1
Genes associated with hypertension (425 genes).

| GENE_ID | GENE | CHR | Patent_ID_number and Priority_date |
|---|---|---|---|
| 57529 | KIAA1318 | X | US 60/819,014 filed on JULY_07_2006 |
| 91851 | CHRDL1 | X | US 60/819,014 filed on JULY_07_2006 |
| 284222 | FLJ34907 | 18 | US 60/819,014 filed on JULY_07_2006 |
| 81469 | OR2G3 | 1 | US 60/819,014 filed on JULY_07_2006 |
| 57559 | STAMBPL1 | 10 | US 60/867,454 filed on NOV_28_2006 |
| 4952 | OCRL | X | US 60/867,454 filed on NOV_28_2006 |
| 6594 | SMARCA1 | X | US 60/867,454 filed on NOV_28_2006 |
| 5354 | PLP1 | X | US 60/819,014 filed on JULY_07_2006 |
| 55787 | CXorf15 | X | US 60/867,454 filed on NOV_28_2006 |
| 392222 | LOC392222 | 8 | US 60/819,014 filed on JULY_07_2006 |
| 5152 | PDE9A | 21 | US 60/867,454 filed on NOV_28_2006 |
| 1312 | COMT | 22 | US 60/867,454 filed on NOV_28_2006 |
| 6197 | RPS6KA3 | X | US 60/867,454 filed on NOV_28_2006 |
| 2892 | GRIA3 | X | US 60/819,014 filed on JULY_07_2006 |
| 152742 | LOC152742 | 4 | US 60/819,014 filed on JULY_07_2006 |
| 29 | ABR | 17 | US 60/867,454 filed on NOV_28_2006 |
| 26047 | CNTNAP2 | 7 | US 11/245,248 filed on NOV2004-AUG2005 |
| 286 | ANK1 | 8 | US 60/819,014 filed on JULY_07_2006 |
| 6480 | ST6GAL1 | 3 | US 60/867,454 filed on NOV_28_2006 |
| 10914 | PAPOLA | 14 | US 60/819,014 filed on JULY_07_2006 |
| 122481 | AK7 | 14 | US 60/867,454 filed on NOV_28_2006 |
| 4772 | NFATC1 | 18 | US 60/819,014 filed on JULY_07_2006 |
| 651082 | LOC651082 | 15 | US 60/867,454 filed on NOV_28_2006 |
| 145567 | TTC7B | 14 | US 60/819,014 filed on JULY_07_2006 |
| 117583 | ALS2CR19 | 2 | US 60/819,014 filed on JULY_07_2006 |
| 96764 | NCOA6IP | 8 | US 60/819,014 filed on JULY_07_2006 |
| 5101 | PCDH9 | 13 | US 60/819,014 filed on JULY_07_2006 |
| 647339 | LOC647339 | 13 | US 60/867,454 filed on NOV_28_2006 |
| 147807 | ZNF524 | 19 | US 60/867,454 filed on NOV_28_2006 |
| 163033 | ZNF579 | 19 | US 60/867,454 filed on NOV_28_2006 |
| 388565 | LOC388565 | 19 | US 60/867,454 filed on NOV_28_2006 |
| 3552 | IL1A | 2 | US 60/819,014 filed on JULY_07_2006 |
| 150468 | FLJ40629 | 2 | US 60/819,014 filed on JULY_07_2006 |
| 651534 | LOC651534 | 12 | US 60/867,454 filed on NOV_28_2006 |
| 5986 | RFNG | 17 | US 60/867,454 filed on NOV_28_2006 |
| 53942 | CNTN5 | 11 | US 60/819,014 filed on JULY_07_2006 |
| 164781 | WDR69 | 2 | US 60/867,454 filed on NOV_28_2006 |
| 1756 | DMD | X | US 60/819,014 filed on JULY_07_2006 |
| 528 | ATP6V1C1 | 8 | US 60/867,454 filed on NOV_28_2006 |
| 79905 | TMC7 | 16 | US 60/867,454 filed on NOV_28_2006 |
| 7988 | ZNF212 | 7 | US 60/867,454 filed on NOV_28_2006 |
| 651362 | LOC651362 | 8 | US 60/867,454 filed on NOV_28_2006 |
| 400576 | FLJ45831 | 17 | US 60/867,454 filed on NOV_28_2006 |
| 55799 | CACNA2D3 | 3 | US 60/867,454 filed on NOV_28_2006 |
| 9104 | RGN | X | US 60/819,014 filed on JULY_07_2006 |
| 3232 | HOXD3 | 2 | US 60/867,454 filed on NOV_28_2006 |
| 400693 | LOC400693 | 19 | US 60/819,014 filed on JULY_07_2006 |
| 255926 | ADAM5 | 8 | US 60/867,454 filed on NOV_28_2006 |
| 10417 | SPON2 | 4 | US 60/867,454 filed on NOV_28_2006 |
| 8749 | ADAM18 | 8 | US 60/867,454 filed on NOV_28_2006 |
| 161176 | C14orf49 | 14 | US 60/819,014 filed on JULY_07_2006 |
| 651311 | LOC651311 | 11 | US 60/867,454 filed on NOV_28_2006 |
| 286094 | LOC286094 | 8 | US 60/867,454 filed on NOV_28_2006 |
| 10178 | ODZ1 | X | US 60/867,454 filed on NOV_28_2006 |
| 79701 | FLJ22222 | 17 | US 60/867,454 filed on NOV_28_2006 |
| 64094 | SMOC2 | 6 | US 60/819,014 filed on JULY_07_2006 |
| 23205 | BG1 | 15 | US 11/245,248 filed on NOV2004-AUG2005 |
| 9658 | ZNF516 | 18 | US 60/867,454 filed on NOV_28_2006 |
| 943 | TNFRSF8 | 1 | US 60/819,014 filed on JULY_07_2006 |
| 161357 | MAMDC1 | 14 | US 60/819,014 filed on JULY_07_2006 |
| 8825 | LIN7A | 12 | US 60/867,454 filed on NOV_28_2006 |
| 3673 | ITGA2 | 5 | US 60/867,454 filed on NOV_28_2006 |
| 51422 | PRKAG2 | 7 | US 60/867,454 filed on NOV_28_2006 |
| 90 | ACVR1 | 2 | US 11/245,248 filed on NOV2004-AUG2005 |
| 491 | ATP2B2 | 3 | US 60/867,454 filed on NOV_28_2006 |
| 1838 | DTNB | 2 | US 60/867,454 filed on NOV_28_2006 |
| 2898 | GRIK2 | 6 | US 60/867,454 filed on NOV_28_2006 |
| 4313 | MMP2 | 16 | US 60/819,014 filed on JULY_07_2006 |
| 5581 | PRKCE | 2 | US 11/245,248 filed on NOV2004-AUG2005 |
| 8499 | PPFIA2 | 12 | US 60/867,454 filed on NOV_28_2006 |
| 9586 | CREB5 | 7 | US 60/867,454 filed on NOV_28_2006 |
| 10046 | CXorf6 | X | US 60/867,454 filed on NOV_28_2006 |
| 10369 | CACNG2 | 22 | US 60/819,014 filed on JULY_07_2006 |
| 11055 | ZPBP | 7 | US 60/867,454 filed on NOV_28_2006 |
| 23233 | SEC15L2 | 2 | US 60/819,014 filed on JULY_07_2006 |
| 25817 | TAFA5 | 22 | US 60/819,014 filed on JULY_07_2006 |
| 55691 | FRMD4A | 10 | US 60/819,014 filed on JULY_07_2006 |
| 60676 | PAPPA2 | 1 | US 60/819,014 filed on JULY_07_2006 |
| 79068 | FTO | 16 | US 60/867,454 filed on NOV_28_2006 |
| 139324 | CXorf43 | X | US 60/867,454 filed on NOV_28_2006 |
| 149986 | C20orf40 | 20 | US 60/819,014 filed on JULY_07_2006 |
| 169044 | COL22A1 | 8 | US 60/819,014 filed on JULY_07_2006 |
| 219578 | FLJ32110 | 7 | US 60/819,014 filed on JULY_07_2006 |
| 441629 | LOC441629 | 12 | US 60/819,014 filed on JULY_07_2006 |
| 648551 | LOC648551 | 22 | US 60/867,454 filed on NOV_28_2006 |
| 387648 | LOC387648 | 10 | US 60/819,014 filed on JULY_07_2006 |
| 1949 | EFNB3 | 17 | US 60/867,454 filed on NOV_28_2006 |
| 84626 | KIAA1862 | 7 | US 60/819,014 filed on JULY_07_2006 |
| 3077 | HFE | 6 | US 60/867,454 filed on NOV_28_2006 |
| 8364 | HIST1H4C | 6 | US 60/867,454 filed on NOV_28_2006 |
| 340156 | LOC340156 | 6 | US 60/819,014 filed on JULY_07_2006 |
| 5536 | PPP5C | 19 | US 60/819,014 filed on JULY_07_2006 |
| 57107 | C6orf210 | 6 | US 60/819,014 filed on JULY_07_2006 |
| 6793 | STK10 | 5 | US 60/867,454 filed on NOV_28_2006 |
| 1902 | EDG2 | 9 | US 60/819,014 filed on JULY_07_2006 |
| 2104 | ESRRG | 1 | US 60/867,454 filed on NOV_28_2006 |
| 6919 | TCEA2 | 20 | US 60/867,454 filed on NOV_28_2006 |
| 286053 | C8orf36 | 8 | US 60/819,014 filed on JULY_07_2006 |
| 23098 | SARM1 | 17 | US 60/819,014 filed on JULY_07_2006 |
| 55843 | ARHGAP15 | 2 | US 60/867,454 filed on NOV_28_2006 |
| 4892 | NRAP | 10 | US 60/867,454 filed on NOV_28_2006 |
| 199731 | IGSF4C | 19 | US 60/867,454 filed on NOV_28_2006 |
| 8621 | CDC2L5 | 7 | US 60/867,454 filed on NOV_28_2006 |
| 5660 | PSAP | 10 | US 60/867,454 filed on NOV_28_2006 |
| 27445 | PCLO | 7 | US 60/867,454 filed on NOV_28_2006 |
| 8829 | NRP1 | 10 | US 60/819,014 filed on JULY_07_2006 |
| 6660 | SOX5 | 12 | US 60/819,014 filed on JULY_07_2006 |
| 50863 | HNT | 11 | US 60/819,014 filed on JULY_07_2006 |
| 773 | CACNA1A | 19 | US 60/819,014 filed on JULY_07_2006 |
| 5211 | PFKL | 21 | US 60/867,454 filed on NOV_28_2006 |
| 10098 | TSPAN5 | 4 | US 60/867,454 filed on NOV_28_2006 |
| 160364 | MICL | 12 | US 60/819,014 filed on JULY_07_2006 |
| 22871 | NLGN1 | 3 | US 60/867,454 filed on NOV_28_2006 |
| 649922 | LOC649922 | 5 | US 60/867,454 filed on NOV_28_2006 |
| 9968 | TNRC11 | X | US 60/819,014 filed on JULY_07_2006 |
| 781 | CACNA2D1 | 7 | US 60/867,454 filed on NOV_28_2006 |
| 4286 | MITF | 3 | US 60/867,454 filed on NOV_28_2006 |
| 10752 | CHL1 | 3 | US 60/819,014 filed on JULY_07_2006 |
| 23705 | IGSF4 | 11 | US 60/867,454 filed on NOV_28_2006 |
| 26085 | KLK13 | 19 | US 60/867,454 filed on NOV_28_2006 |
| 151473 | SLC16A14 | 2 | US 60/819,014 filed on JULY_07_2006 |
| 340596 | LHFPL1 | X | US 60/819,014 filed on JULY_07_2006 |
| 391353 | LOC391353 | 2 | US 60/819,014 filed on JULY_07_2006 |
| 392533 | LOC392533 | X | US 60/819,014 filed on JULY_07_2006 |
| 442237 | LOC442237 | 6 | US 60/867,454 filed on NOV_28_2006 |
| 23523 | CABIN1 | 22 | US 60/867,454 filed on NOV_28_2006 |
| 648941 | LOC648941 | 22 | US 60/867,454 filed on NOV_28_2006 |
| 4255 | MGMT | 10 | US 60/867,454 filed on NOV_28_2006 |
| 649173 | LOC649173 | 17 | US 60/867,454 filed on NOV_28_2006 |
| 57143 | ADCK1 | 14 | US 60/819,014 filed on JULY_07_2006 |
| 2048 | EPHB2 | 1 | US 60/867,454 filed on NOV_28_2006 |
| 55227 | LRRC1 | 6 | US 60/819,014 filed on JULY_07_2006 |
| 137868 | SGCZ | 8 | US 11/245,248 filed on NOV2004-AUG2005 |
| 651758 | LOC651758 | 2 | US 60/867,454 filed on NOV_28_2006 |
| 63905 | MANBAL | 20 | US 60/867,454 filed on NOV_28_2006 |
| 3479 | IGF1 | 12 | US 60/819,014 filed on JULY_07_2006 |
| 5475 | PPEF1 | X | US 60/867,454 filed on NOV_28_2006 |
| 1823 | DSC1 | 18 | US 60/819,014 filed on JULY_07_2006 |
| 7748 | ZNF195 | 11 | US 60/867,454 filed on NOV_28_2006 |
| 23129 | PLXND1 | 3 | US 60/867,454 filed on NOV_28_2006 |
| 200150 | PLD5 | 1 | US 60/867,454 filed on NOV_28_2006 |
| 7010 | TEK | 9 | US 60/867,454 filed on NOV_28_2006 |
| 26280 | IL1RAPL2 | X | US 60/867,454 filed on NOV_28_2006 |
| 2175 | FANCA | 16 | US 60/867,454 filed on NOV_28_2006 |
| 55869 | HDAC8 | X | US 60/819,014 filed on JULY_07_2006 |
| 84623 | KIRREL3 | 11 | US 60/867,454 filed on NOV_28_2006 |
| 81608 | FIP1L1 | 4 | US 60/819,014 filed on JULY_07_2006 |
| 9213 | XPR1 | 1 | US 60/867,454 filed on NOV_28_2006 |
| 9265 | PSCD3 | 7 | US 60/867,454 filed on NOV_28_2006 |
| 114781 | BTBD9 | 6 | US 60/867,454 filed on NOV_28_2006 |
| 401398 | LOC401398 | 7 | US 60/867,454 filed on NOV_28_2006 |
| 2334 | AFF2 | X | US 60/867,454 filed on NOV_28_2006 |
| 84056 | KATNAL1 | 13 | US 60/867,454 filed on NOV_28_2006 |
| 610 | HCN2 | 19 | US 60/867,454 filed on NOV_28_2006 |
| 2900 | GRIK4 | 11 | US 60/867,454 filed on NOV_28_2006 |
| 6792 | CDKL5 | X | US 60/819,014 filed on JULY_07_2006 |
| 126917 | LOC126917 | 1 | US 60/867,454 filed on NOV_28_2006 |
| 154215 | TCBA1 | 6 | US 11/245,248 filed on NOV2004-AUG2005 |
| 158038 | LRRN6C | 9 | US 11/245,248 filed on NOV2004-AUG2005 |
| 158521 | FMR1NB | X | US 60/867,454 filed on NOV_28_2006 |
| 203062 | TSNARE1 | 8 | US 60/867,454 filed on NOV_28_2006 |
| 254065 | BRODL | X | US 60/819,014 filed on JULY_07_2006 |
| 642216 | LOC642216 | 5 | US 60/867,454 filed on NOV_28_2006 |
| 219743 | TYSND1 | 10 | US 60/819,014 filed on JULY_07_2006 |
| 389293 | LOC389293 | 5 | US 60/819,014 filed on JULY_07_2006 |
| 9705 | ST18 | 8 | US 60/867,454 filed on NOV_28_2006 |
| 55326 | AGPAT5 | 8 | US 60/867,454 filed on NOV_28_2006 |
| 23613 | PRKCBP1 | 20 | US 60/819,014 filed on JULY_07_2006 |
| 83716 | CRISPLD2 | 16 | US 60/867,454 filed on NOV_28_2006 |
| 653983 | LOC653983 | 4 | US 60/867,454 filed on NOV_28_2006 |
| 149134 | LOC149134 | 1 | US 60/819,014 filed on JULY_07_2006 |
| 170679 | PSORS1C1 | 6 | US 60/867,454 filed on NOV_28_2006 |
| 23041 | KIAA1040 | 12 | US 60/867,454 filed on NOV_28_2006 |
| 222255 | ATXN7L4 | 7 | US 60/867,454 filed on NOV_28_2006 |
| 644055 | LOC644055 | 2 | US 60/867,454 filed on NOV_28_2006 |
| 392670 | LOC392670 | 7 | US 60/867,454 filed on NOV_28_2006 |
| 440193 | LOC440193 | 14 | US 60/819,014 filed on JULY_07_2006 |
| 391475 | LOC391475 | 2 | US 60/867,454 filed on NOV_28_2006 |
| 9759 | HDAC4 | 2 | US 60/867,454 filed on NOV_28_2006 |
| 9962 | SLC23A2 | 20 | US 60/867,454 filed on NOV_28_2006 |
| 10666 | CD226 | 18 | US 60/867,454 filed on NOV_28_2006 |
| 3720 | JARID2 | 6 | US 60/867,454 filed on NOV_28_2006 |
| 1826 | DSCAM | 21 | US 60/819,014 filed on JULY_07_2006 |
| 285195 | SLC9A9 | 3 | US 60/819,014 filed on JULY_07_2006 |
| 392456 | LOC392456 | X | US 60/819,014 filed on JULY_07_2006 |
| 57624 | KIAA1486 | 2 | US 60/867,454 filed on NOV_28_2006 |
| 649120 | LOC649120 | 5 | US 60/867,454 filed on NOV_28_2006 |
| 386617 | KCTD8 | 4 | US 60/867,454 filed on NOV_28_2006 |
| 199920 | C1orf168 | 1 | US 60/867,454 filed on NOV_28_2006 |
| 10186 | LHFP | 13 | US 60/867,454 filed on NOV_28_2006 |
| 51084 | CRYL1 | 13 | US 60/819,014 filed on JULY_07_2006 |
| 959 | TNFSF5 | X | US 60/819,014 filed on JULY_07_2006 |
| 11278 | KLF12 | 13 | US 60/819,014 filed on JULY_07_2006 |
| 55289 | ACOXL | 2 | US 60/819,014 filed on JULY_07_2006 |
| 8974 | P4HA2 | 5 | US 60/867,454 filed on NOV_28_2006 |
| 64839 | FBXL17 | 5 | US 11/245,248 filed on NOV2004-AUG2005 |
| 580 | BARD1 | 2 | US 11/245,248 filed on NOV2004-AUG2005 |
| 647489 | LOC647489 | 18 | US 60/867,454 filed on NOV_28_2006 |
| 2272 | FHIT | 3 | US 60/819,014 filed on JULY_07_2006 |
| 4745 | NELL1 | 11 | US 11/245,248 filed on NOV2004-AUG2005 |
| 64420 | SUSD1 | 9 | US 60/867,454 filed on NOV_28_2006 |
| 441496 | LOC441496 | X | US 60/819,014 filed on JULY_07_2006 |
| 442457 | LOC442457 | X | US 60/819,014 filed on JULY_07_2006 |
| 122046 | MGC40178 | 13 | US 60/867,454 filed on NOV_28_2006 |
| 27328 | PCDH11X | X | US 60/819,014 filed on JULY_07_2006 |
| 81849 | ST6GALNAC5 | 1 | US 60/867,454 filed on NOV_28_2006 |
| 272 | AMPD3 | 11 | US 60/867,454 filed on NOV_28_2006 |
| 84000 | TMPRSS13 | 11 | US 60/867,454 filed on NOV_28_2006 |
| 3990 | LIPC | 15 | US 60/867,454 filed on NOV_28_2006 |
| 139163 | LOC139163 | X | US 60/867,454 filed on NOV_28_2006 |
| 390683 | LOC390683 | 16 | US 60/819,014 filed on JULY_07_2006 |
| 1630 | DCC | 18 | US 60/819,014 filed on JULY_07_2006 |
| 10642 | IMP-1 | 17 | US 60/867,454 filed on NOV_28_2006 |
| 9645 | MICAL2 | 11 | US 60/867,454 filed on NOV_28_2006 |
| 26059 | CAST1 | 3 | US 60/867,454 filed on NOV_28_2006 |
| 57540 | PTCHD2 | 1 | US 60/867,454 filed on NOV_28_2006 |
| 79611 | FLJ21963 | 12 | US 60/819,014 filed on JULY_07_2006 |
| 10345 | TRDN | 6 | US 60/819,014 filed on JULY_07_2006 |
| 8548 | BLZF1 | 1 | US 60/867,454 filed on NOV_28_2006 |
| 5530 | PPP3CA | 4 | US 60/867,454 filed on NOV_28_2006 |
| 375449 | LOC375449 | 5 | US 60/819,014 filed on JULY_07_2006 |
| 57533 | TBC1D14 | 4 | US 60/819,014 filed on JULY_07_2006 |
| 441062 | LOC441062 | 5 | US 60/819,014 filed on JULY_07_2006 |
| 3557 | IL1RN | 2 | US 60/867,454 filed on NOV_28_2006 |
| 5144 | PDE4D | 5 | US 60/867,454 filed on NOV_28_2006 |
| 23274 | KIAA0350 | 16 | US 60/867,454 filed on NOV_28_2006 |
| 341350 | OVCH1 | 12 | US 60/819,014 filed on JULY_07_2006 |
| 27075 | TSPAN13 | 7 | US 60/867,454 filed on NOV_28_2006 |
| 7068 | THRB | 3 | US 60/867,454 filed on NOV_28_2006 |
| 9843 | HEPH | X | US 60/867,454 filed on NOV_28_2006 |
| 84629 | KIAA1856 | 7 | US 60/819,014 filed on JULY_07_2006 |
| 152330 | CNTN4 | 3 | US 60/819,014 filed on JULY_07_2006 |
| 253582 | C6orf191 | 6 | US 60/867,454 filed on NOV_28_2006 |
| 408 | ARRB1 | 11 | US 60/867,454 filed on NOV_28_2006 |
| 126859 | C1orf125 | 1 | US 60/867,454 filed on NOV_28_2006 |
| 23779 | ARHGAP8 | 22 | US 60/867,454 filed on NOV_28_2006 |
| 651344 | LOC651344 | 11 | US 60/867,454 filed on NOV_28_2006 |
| 85302 | FBF1 | 17 | US 60/867,454 filed on NOV_28_2006 |
| 7204 | TRIO | 5 | US 60/867,454 filed on NOV_28_2006 |
| 26577 | PCOLCE2 | 3 | US 60/867,454 filed on NOV_28_2006 |
| 5286 | PIK3C2A | 11 | US 60/867,454 filed on NOV_28_2006 |
| 27253 | PCDH17 | 13 | US 60/819,014 filed on JULY_07_2006 |
| 90293 | KLHL13 | X | US 60/819,014 filed on JULY_07_2006 |
| 347694 | ECEL1P2 | 2 | US 60/867,454 filed on NOV_28_2006 |
| 1607 | DGKB | 7 | US 60/819,014 filed on JULY_07_2006 |
| 463 | ATBF1 | 16 | US 60/867,454 filed on NOV_28_2006 |
| 5119 | PCOLN3 | 16 | US 60/867,454 filed on NOV_28_2006 |
| 124044 | MGC26885 | 16 | US 60/867,454 filed on NOV_28_2006 |
| 283455 | KSR2 | 12 | US 60/867,454 filed on NOV_28_2006 |
| 2185 | PTK2B | 8 | US 60/819,014 filed on JULY_07_2006 |
| 254827 | NAALADL2 | 3 | US 60/819,014 filed on JULY_07_2006 |
| 79446 | MGC4645 | 14 | US 11/245,248 filed on NOV2004-AUG2005 |
| 3760 | KCNJ3 | 2 | US 60/819,014 filed on JULY_07_2006 |
| 284186 | TMEM105 | 17 | US 60/867,454 filed on NOV_28_2006 |
| 388790 | LOC388790 | 20 | US 60/819,014 filed on JULY_07_2006 |
| 651301 | LOC651301 | 3 | US 60/867,454 filed on NOV_28_2006 |
| 22987 | SV2C | 5 | US 60/867,454 filed on NOV_28_2006 |
| 254170 | FBXO33 | 14 | US 60/867,454 filed on NOV_28_2006 |
| 11142 | PKIG | 20 | US 60/867,454 filed on NOV_28_2006 |
| 5167 | ENPP1 | 6 | US 60/867,454 filed on NOV_28_2006 |
| 29119 | CTNNA3 | 10 | US 11/245,248 filed on NOV2004-AUG2005 |
| 5087 | PBX1 | 1 | US 60/867,454 filed on NOV_28_2006 |
| 1600 | DAB1 | 1 | US 11/245,248 filed on NOV2004-AUG2005 |
| 1770 | DNAH9 | 17 | US 60/819,014 filed on JULY_07_2006 |
| 11141 | IL1RAPL1 | X | US 60/819,014 filed on JULY_07_2006 |
| 23005 | MAPKBP1 | 15 | US 60/867,454 filed on NOV_28_2006 |
| 26984 | SEC22L2 | 3 | US 60/867,454 filed on NOV_28_2006 |
| 2888 | GRB14 | 2 | US 60/867,454 filed on NOV_28_2006 |
| 5651 | PRSS7 | 21 | US 60/867,454 filed on NOV_28_2006 |
| 9628 | RGS6 | 14 | US 60/867,454 filed on NOV_28_2006 |
| 649004 | LOC649004 | 2 | US 60/867,454 filed on NOV_28_2006 |
| 6928 | TCF2 | 17 | US 60/867,454 filed on NOV_28_2006 |
| 8228 | DXS1283E | X | US 60/819,014 filed on JULY_07_2006 |
| 84941 | HSH2D | 19 | US 60/867,454 filed on NOV_28_2006 |
| 648814 | LOC648814 | 8 | US 60/867,454 filed on NOV_28_2006 |
| 23072 | HECW1 | 7 | US 60/867,454 filed on NOV_28_2006 |
| 7498 | XDH | 2 | US 60/867,454 filed on NOV_28_2006 |
| 79789 | CLMN | 14 | US 60/867,454 filed on NOV_28_2006 |
| 1012 | CDH13 | 16 | US 11/245,248 filed on NOV2004-AUG2005 |
| 4685 | NCAM2 | 21 | US 60/819,014 filed on JULY_07_2006 |
| 11043 | MID2 | X | US 60/819,014 filed on JULY_07_2006 |
| 51097 | CGI-49 | 1 | US 60/819,014 filed on JULY_07_2006 |
| 54777 | C10orf92 | 10 | US 60/867,454 filed on NOV_28_2006 |
| 647525 | LOC647525 | 10 | US 60/867,454 filed on NOV_28_2006 |
| 650079 | LOC650079 | 9 | US 60/867,454 filed on NOV_28_2006 |
| 104 | ADARB1 | 21 | US 60/867,454 filed on NOV_28_2006 |
| 7402 | UTRN | 6 | US 11/245,248 filed on NOV2004-AUG2005 |
| 57214 | KIAA1199 | 15 | US 60/819,014 filed on JULY_07_2006 |
| 23012 | STK38L | 12 | US 60/867,454 filed on NOV_28_2006 |
| 642172 | LOC642172 | 13 | US 60/867,454 filed on NOV_28_2006 |
| 28667 | TRAV16 | 14 | US 60/867,454 filed on NOV_28_2006 |
| 7174 | TPP2 | 13 | US 60/867,454 filed on NOV_28_2006 |
| 641864 | LOC641864 | 7 | US 60/867,454 filed on NOV_28_2006 |
| 170692 | ADAMTS18 | 16 | US 60/819,014 filed on JULY_07_2006 |
| 652214 | LOC652214 | 2 | US 60/867,454 filed on NOV_28_2006 |
| 4281 | MID1 | X | US 60/867,454 filed on NOV_28_2006 |
| 4045 | LSAMP | 3 | US 60/819,014 filed on JULY_07_2006 |
| 54868 | TMEM104 | 17 | US 60/867,454 filed on NOV_28_2006 |
| 51696 | HECA | 6 | US 60/867,454 filed on NOV_28_2006 |
| 2903 | GRIN2A | 16 | US 60/867,454 filed on NOV_28_2006 |
| 6862 | T | 6 | US 11/245,248 filed on NOV2004-AUG2005 |
| 5332 | PLCB4 | 20 | US 60/867,454 filed on NOV_28_2006 |
| 23362 | PSD3 | 8 | US 11/245,248 filed on NOV2004-AUG2005 |
| 56999 | ADAMTS9 | 3 | US 60/819,014 filed on JULY_07_2006 |
| 220108 | FLJ30707 | 13 | US 60/867,454 filed on NOV_28_2006 |
| 55698 | FLJ10324 | 7 | US 60/867,454 filed on NOV_28_2006 |
| 55658 | RNF126 | 19 | US 60/867,454 filed on NOV_28_2006 |
| 9731 | GlyBP | 1 | US 60/867,454 filed on NOV_28_2006 |
| 2736 | GLI2 | 2 | US 60/819,014 filed on JULY_07_2006 |
| 154386 | C6orf195 | 6 | US 60/819,014 filed on JULY_07_2006 |
| 401548 | SNX30 | 9 | US 60/867,454 filed on NOV_28_2006 |
| 23095 | KIF1B | 1 | US 60/867,454 filed on NOV_28_2006 |
| 4872 | NPM1P3 | 16 | US 60/819,014 filed on JULY_07_2006 |
| 5579 | PRKCB1 | 16 | US 60/819,014 filed on JULY_07_2006 |
| 23200 | ATP11B | 3 | US 60/867,454 filed on NOV_28_2006 |
| 129684 | CNTNAP5 | 2 | US 60/819,014 filed on JULY_07_2006 |
| 414260 | C10orf136 | 10 | US 60/867,454 filed on NOV_28_2006 |
| 648118 | LOC648118 | X | US 60/867,454 filed on NOV_28_2006 |
| 2863 | GPR39 | 2 | US 60/819,014 filed on JULY_07_2006 |
| 6563 | SLC14A1 | 18 | US 60/867,454 filed on NOV_28_2006 |
| 64072 | CDH23 | 10 | US 60/819,014 filed on JULY_07_2006 |
| 151742 | PPM1L | 3 | US 60/867,454 filed on NOV_28_2006 |
| 5077 | PAX3 | 2 | US 60/819,014 filed on JULY_07_2006 |
| 441822 | LOC441822 | 18 | US 60/819,014 filed on JULY_07_2006 |
| 2742 | GLRA2 | X | US 60/867,454 filed on NOV_28_2006 |
| 9957 | HS3ST1 | 4 | US 60/867,454 filed on NOV_28_2006 |
| 200132 | TCTEX1D1 | 1 | US 60/867,454 filed on NOV_28_2006 |
| 9899 | SV2B | 15 | US 60/867,454 filed on NOV_28_2006 |
| 10954 | PDIA5 | 3 | US 60/867,454 filed on NOV_28_2006 |
| 11102 | RPP14 | 3 | US 60/819,014 filed on JULY_07_2006 |
| 83893 | SPATA16 | 3 | US 60/867,454 filed on NOV_28_2006 |
| 1962 | EHHADH | 3 | US 60/867,454 filed on NOV_28_2006 |
| 7290 | HIRA | 22 | US 60/819,014 filed on JULY_07_2006 |
| 6529 | SLC6A1 | 3 | US 60/867,454 filed on NOV_28_2006 |
| 285498 | LOC285498 | 4 | US 60/867,454 filed on NOV_28_2006 |
| 2917 | GRM7 | 3 | US 11/245,248 filed on NOV2004-AUG2005 |
| 79772 | MCTP1 | 5 | US 60/867,454 filed on NOV_28_2006 |
| 283682 | LOC283682 | 15 | US 60/867,454 filed on NOV_28_2006 |
| 651419 | LOC651419 | 5 | US 60/867,454 filed on NOV_28_2006 |
| 9037 | SEMA5A | 5 | US 60/819,014 filed on JULY_07_2006 |
| 9071 | CLDN10 | 13 | US 60/819,014 filed on JULY_07_2006 |
| 6522 | SLC4A2 | 7 | US 60/819,014 filed on JULY_07_2006 |
| 26146 | TRAF3IP1 | 2 | US 11/245,248 filed on NOV2004-AUG2005 |
| 91582 | MGC52010 | 22 | US 60/819,014 filed on JULY_07_2006 |
| 123355 | LRRC28 | 15 | US 60/867,454 filed on NOV_28_2006 |
| 22874 | PLEKHA6 | 1 | US 60/867,454 filed on NOV_28_2006 |
| 57492 | ARID1B | 6 | US 60/819,014 filed on JULY_07_2006 |
| 55714 | ODZ3 | 4 | US 60/867,454 filed on NOV_28_2006 |
| 1948 | EFNB2 | 13 | US 60/867,454 filed on NOV_28_2006 |
| 128553 | ZNF218 | 20 | US 60/867,454 filed on NOV_28_2006 |
| 28232 | SLCO3A1 | 15 | US 11/245,248 filed on NOV2004-AUG2005 |
| 81792 | ADAMTS12 | 5 | US 11/245,248 filed on NOV2004-AUG2005 |
| 5794 | PTPRH | 19 | US 60/819,014 filed on JULY_07_2006 |
| 8828 | NRP2 | 2 | US 60/819,014 filed on JULY_07_2006 |
| 8997 | HAPIP | 3 | US 11/245,248 filed on NOV2004-AUG2005 |
| 9369 | NRXN3 | 14 | US 11/245,248 filed on NOV2004-AUG2005 |
| 51751 | HIGD1B | 17 | US 60/867,454 filed on NOV_28_2006 |
| 114792 | KIAA1900 | 6 | US 60/819,014 filed on JULY_07_2006 |
| 154796 | AMOT | X | US 60/819,014 filed on JULY_07_2006 |
| 15Pt | MGC34646 | 8 | US 60/867,454 filed on NOV_28_2006 |
| 400955 | LOC400955 | 2 | US 60/867,454 filed on NOV_28_2006 |
| 6870 | TACR3 | 4 | US 60/867,454 filed on NOV_28_2006 |
| 8139 | GAN | 16 | US 60/867,454 filed on NOV_28_2006 |
| 8760 | CDS2 | 20 | US 60/867,454 filed on NOV_28_2006 |
| 64759 | TNS3 | 7 | US 60/867,454 filed on NOV_28_2006 |
| 1807 | DPYS | 8 | US 60/867,454 filed on NOV_28_2006 |
| 152189 | CKLFSF8 | 3 | US 60/819,014 filed on JULY_07_2006 |
| 433 | ASGR2 | 17 | US 60/867,454 filed on NOV_28_2006 |
| 3782 | KCNN3 | 1 | US 60/867,454 filed on NOV_28_2006 |
| 3607 | FOXK2 | 17 | US 60/867,454 filed on NOV_28_2006 |
| 25913 | POT1 | 7 | US 60/867,454 filed on NOV_28_2006 |
| 57419 | SLC24A3 | 20 | US 60/819,014 filed on JULY_07_2006 |
| 9180 | OSMR | 5 | US 60/819,014 filed on JULY_07_2006 |
| 1002 | CDH4 | 20 | US 60/867,454 filed on NOV_28_2006 |
| 57186 | C20orf74 | 20 | US 60/867,454 filed on NOV_28_2006 |
| 11095 | ADAMTS8 | 11 | US 60/867,454 filed on NOV_28_2006 |
| 55733 | MART2 | 1 | US 60/819,014 filed on JULY_07_2006 |
| 124045 | C16orf55 | 16 | US 60/867,454 filed on NOV_28_2006 |
| 441284 | LOC441284 | 7 | US 60/819,014 filed on JULY_07_2006 |
| 54840 | APTX | 9 | US 60/867,454 filed on NOV_28_2006 |
| 1010 | CDH12 | 5 | US 60/867,454 filed on NOV_28_2006 |
| 2918 | GRM8 | 7 | US 60/867,454 filed on NOV_28_2006 |
| 4211 | MEIS1 | 2 | US 60/819,014 filed on JULY_07_2006 |
| 9019 | MPZL1 | 1 | US 60/867,454 filed on NOV_28_2006 |
| 10246 | SLC17A2 | 6 | US 60/819,014 filed on JULY_07_2006 |
| 23170 | KIAA0153 | 22 | US 60/867,454 filed on NOV_28_2006 |
| 22999 | RIMS1 | 6 | US 60/867,454 filed on NOV_28_2006 |
| 650912 | LOC650912 | 13 | US 60/867,454 filed on NOV_28_2006 |
| 11262 | SP140 | 2 | US 60/819,014 filed on JULY_07_2006 |
| 9201 | DCAMKL1 | 13 | US 60/867,454 filed on NOV_28_2006 |
| 253558 | LYCAT | 2 | US 60/867,454 filed on NOV_28_2006 |
| 412 | STS | X | US 60/867,454 filed on NOV_28_2006 |
| 10057 | ABCC5 | 3 | US 60/819,014 filed on JULY_07_2006 |
| 51760 | SYT17 | 16 | US 60/867,454 filed on NOV_28_2006 |
| 392517 | LOC392517 | X | US 60/819,014 filed on JULY_07_2006 |
| 494118 | SPANX-N1 | X | US 60/867,454 filed on NOV_28_2006 |
| 1124 | CHN2 | 7 | US 60/867,454 filed on NOV_28_2006 |
| 648089 | LOC648089 | 5 | US 60/867,454 filed on NOV_28_2006 |
| 150946 | LOC150946 | 2 | US 60/819,014 filed on JULY_07_2006 |
| 152485 | LOC152485 | 4 | US 60/867,454 filed on NOV_28_2006 |
| 5218 | PFTK1 | 7 | US 60/867,454 filed on NOV_28_2006 |
| 245973 | ATP6V1C2 | 2 | US 60/867,454 filed on NOV_28_2006 |
| 6196 | RPS6KA2 | 6 | US 11/245,248 filed on NOV2004-AUG2005 |
| 137695 | FLJ32370 | 8 | US 60/819,014 filed on JULY_07_2006 |
| 51360 | MBTPS2 | X | US 60/819,014 filed on JULY_07_2006 |
| 80731 | KIAA1679 | 2 | US 60/867,454 filed on NOV_28_2006 |
| 5797 | PTPRM | 18 | US 11/245,248 filed on NOV2004-AUG2005 |
| 6483 | SIAT4B | 16 | US 60/819,014 filed on JULY_07_2006 |
| 26074 | C20orf26 | 20 | US 60/867,454 filed on NOV_28_2006 |
| 84708 | LNX | 4 | US 60/819,014 filed on JULY_07_2006 |
| 649035 | LOC649035 | 12 | US 60/867,454 filed on NOV_28_2006 |
| 9111 | NMI | 2 | US 60/867,454 filed on NOV_28_2006 |
| 83857 | TMTC1 | 12 | US 60/819,014 filed on JULY_07_2006 |
| 92291 | CAPN13 | 2 | US 60/819,014 filed on JULY_07_2006 |
| 344595 | LOC344595 | 3 | US 60/867,454 filed on NOV_28_2006 |
| 2066 | ERBB4 | 2 | US 11/245,248 filed on NOV2004-AUG2005 |
| 647947 | LOC647947 | 4 | US 60/867,454 filed on NOV_28_2006 |
| 1395 | CRHR2 | 7 | US 60/867,454 filed on NOV_28_2006 |
| 2139 | EYA2 | 20 | US 60/819,014 filed on JULY_07_2006 |
| 151258 | FLJ39822 | 2 | US 60/867,454 filed on NOV_28_2006 |
| 1385 | CREB1 | 2 | US 60/819,014 filed on JULY_07_2006 |
| 5688 | PSMA7 | 20 | US 60/819,014 filed on JULY_07_2006 |
| 10052 | GJA7 | 17 | US 60/867,454 filed on NOV_28_2006 |
| 55742 | PARVA | 11 | US 60/867,454 filed on NOV_28_2006 |
| 126410 | FLJ39501 | 19 | US 60/819,014 filed on JULY_07_2006 |

TABLE 2
SNP markers with the strongest association with HT in the individual marker analysis.
The analysis is based on 140 HT cases and 182 healthy controls from East Finland.

| | Gene locus | | | | | | | |
|---|---|---|---|---|---|---|---|---|
| | and | | | | | | | |
| dbSNP rs ID | Gene ID | | Chromosome | Position | Variats | Minor Allele | Allele X2 | Odds ratio |
| rs901185 | FLJ34907 | 284222 | 18 | 10844509 | ‘C/T’ | G | 28.03 | 0.16 |
| rs7328290 | | | 13 | 71394581 | ‘A/G’ | A | 21.53 | 3.16 |
| rs12379069 | | | 9 | 24065368 | ‘C/T’ | A | 19.33 | 2.03 |
| rs7931411 | CNTN5 | 53942 | 11 | 99259555 | ‘A/G’ | G | 19.25 | 2.03 |
| rs7814270 | | | 8 | 33590441 | ‘A/G’ | A | 18.58 | 2.01 |
| rs10511739 | | | 9 | 24081342 | ‘A/G’ | G | 18.56 | 2.00 |
| rs1910236 | | | 3 | 59409460 | ‘C/T’ | A | 17.98 | 1.97 |
| rs7333943 | | | 13 | 58515848 | ‘G/T’ | C | 17.87 | 2.71 |
| rs1938684 | | | 11 | 68986287 | ‘C/T’ | A | 17.30 | 2.21 |
| rs2209902 | PCDH9 | 5101 | 13 | 66600753 | ‘C/T’ | A | 17.12 | 2.34 |
| rs6812187 | | | 4 | 21295968 | ‘C/T’ | G | 16.94 | 1.99 |
| rs2824669 | | | 21 | 18457462 | ‘A/C’ | C | 16.88 | 1.95 |
| rs1395000 | | | 4 | 85197861 | ‘A/G’ | G | 16.85 | 1.96 |
| rs2290999 | LOC400693 | 400693 | 19 | 42830727 | ‘A/G’ | G | 16.83 | 2.29 |
| rs10107668 | | | 8 | 33643721 | ‘C/T’ | G | 16.48 | 1.97 |
| rs2012192 | C14orf49 | 161176 | 14 | 94998087 | ‘A/G’ | A | 16.37 | 0.42 |
| rs7995254 | PCDH9 | 5101 | 13 | 66501746 | ‘C/T’ | G | 16.31 | 2.56 |
| rs4708483 | SMOC2 | 64094 | 6 | 168865533 | ‘C/T’ | A | 15.87 | 2.11 |
| rs3813577 | BG1 | 23205 | 15 | 76314308 | ‘A/G’ | G | 15.84 | 0.51 |
| rs501525 | TNFRSF8 | 943 | 1 | 12115071 | ‘A/G’ | G | 15.77 | 1.89 |
| rs2504070 | | | 6 | 152177087 | ‘C/T’ | A | 15.65 | 1.99 |
| rs17517037 | PCDH9 | 5101 | 13 | 66597199 | ‘C/T’ | A | 15.61 | 2.15 |
| rs17560594 | MAMDC1 | 161357 | 14 | 46530953 | ‘C/T’ | G | 15.57 | 2.38 |
| rs3913663 | | | 4 | 21283866 | ‘G/T’ | C | 15.51 | 1.96 |
| rs6896456 | | | 5 | 134605656 | ‘A/G’ | A | 15.38 | 2.94 |
| rs1394139 | | | 4 | 21283327 | ‘C/T’ | G | 15.30 | 1.95 |
| rs1370923 | | | 2 | 222825345 | ‘A/G’ | A | 15.23 | 2.54 |
| rs7097635 | LOC387648 | 387648 | 10 | 31000212 | ‘A/G’ | G | 15.09 | 1.86 |
| rs915251 | | | 6 | 107579760 | ‘A/G’ | A | 15.06 | 1.86 |
| rs10402423 | | | 19 | 1497180 | ‘A/G’ | A | 15.06 | 2.81 |
| rs12673933 | CNTNAP2 | 26047 | 7 | 147441753 | ‘C/T’ | G | 15.06 | 0.49 |
| rs731489 | KIAA1862 | 84626 | 7 | 148798930 | ‘A/G’ | A | 15.05 | 1.89 |
| rs10518621 | | | 4 | 134122347 | ‘C/T’ | A | 15.00 | 0.44 |
| rs1461656 | LOC340156 | 340156 | 6 | 2661859 | ‘A/G’ | A | 14.87 | 0.31 |
| rs759290 | PPP5C | 5536 | 19 | 51583951 | ‘C/T’ | G | 14.85 | 0.51 |
| rs6568470 | C6orf210 | 57107 | 6 | 107607740 | ‘A/G’ | A | 14.84 | 1.96 |
| rs1453590 | CNTN5 | 53942 | 11 | 99271543 | ‘A/C’ | A | 14.81 | 0.49 |
| rs3739709 | EDG2 | 1902 | 9 | 110717409 | ‘C/T’ | A | 14.78 | 0.43 |
| rs4881232 | | | 10 | 3935400 | ‘A/C’ | C | 14.75 | 1.85 |
| rs7901450 | | | 10 | 120200634 | ‘G/T’ | C | 14.69 | 1.86 |
| rs9828674 | | | 3 | 64668140 | ‘G/T’ | C | 14.54 | 1.93 |
| rs2239908 | SARM1 | 23098 | 17 | 23749392 | ‘C/T’ | A | 14.52 | 0.53 |
| rs3820623 | | | 1 | 224413997 | ‘C/T’ | A | 14.50 | 0.46 |
| rs7984277 | | | 13 | 57299434 | ‘A/G’ | G | 14.29 | 4.04 |
| rs6433781 | | | 2 | 180040816 | ‘C/T’ | G | 14.29 | 0.16 |
| rs2167163 | | | 18 | 73004290 | ‘A/G’ | A | 14.28 | 3.19 |
| rs6534907 | | | 4 | 134100931 | ‘C/T’ | G | 14.24 | 0.51 |
| rs2836079 | | | 21 | 38276905 | ‘C/T’ | G | 14.24 | 0.50 |
| rs11936235 | LOC152742 | 152742 | 4 | 13794650 | ‘C/T’ | A | 14.24 | 2.71 |
| rs7815570 | | | 8 | 135966827 | ‘C/T’ | A | 14.13 | 1.98 |
| rs1389626 | LOC387648 | 387648 | 10 | 31011114 | ‘A/G’ | G | 14.12 | 1.82 |
| rs3780869 | NRP1 | 8829 | 10 | 33587471 | ‘A/G’ | A | 14.11 | 0.21 |
| rs16896934 | | | 4 | 17992019 | ‘C/T’ | G | 14.09 | 0.38 |
| rs4848300 | | | 2 | 113244137 | ‘C/T’ | G | 14.04 | 0.52 |
| rs3922562 | SOX5 | 6660 | 12 | 24250132 | ‘C/T’ | A | 14.03 | 0.49 |
| rs574322 | HNT | 50863 | 11 | 131326210 | ‘C/T’ | A | 14.01 | 2.03 |
| rs2302080 | CACNA1A | 773 | 19 | 13217380 | ‘C/T’ | G | 13.99 | 1.82 |
| rs906236 | | | 10 | 30891225 | ‘A/C’ | A | 13.93 | 1.90 |
| rs686148 | MICL | 160364 | 12 | 10020961 | ‘C/T’ | A | 13.93 | 0.37 |
| rs2214552 | | | 7 | 19548460 | ‘C/T’ | G | 13.93 | 1.85 |
| rs7088506 | LOC387648 | 387648 | 10 | 31022758 | ‘C/T’ | G | 13.92 | 1.85 |
| rs17561 | IL1A | 3552 | 2 | 113253454 | ‘G/T’ | A | 13.88 | 0.52 |
| rs12681358 | COL22A1 | 169044 | 8 | 139794900 | ‘C/T’ | A | 13.88 | 2.27 |
| rs2027993 | SARM1 | 23098 | 17 | 23731073 | ‘A/C’ | A | 13.87 | 0.54 |
| rs6844871 | | | 4 | 162268685 | ‘C/T’ | A | 13.81 | 1.86 |
| rs4960948 | | | 8 | 87165300 | ‘A/G’ | G | 13.78 | 1.81 |
| rs1549118 | ADCK1 | 57143 | 14 | 77449437 | ‘C/T’ | A | 13.71 | 0.51 |
| rs1538549 | | | 1 | 191736712 | ‘A/G’ | A | 13.71 | 2.21 |
| rs7835385 | COL22A1 | 169044 | 8 | 139778987 | ‘G/T’ | C | 13.68 | 2.27 |
| rs1883632 | LRRC1 | 55227 | 6 | 53888391 | ‘A/G’ | A | 13.67 | 1.82 |
| rs17119719 | SGCZ | 137868 | 8 | 14436692 | ‘C/T’ | G | 13.67 | 1.81 |
| rs741231 | PPP5C | 5536 | 19 | 51586003 | ‘A/C’ | A | 13.65 | 0.48 |
| rs7098281 | | | 10 | 92897589 | ‘G/T’ | C | 13.64 | 0.43 |
| rs7151137 | | | 14 | 94278193 | ‘C/T’ | A | 13.63 | 0.36 |
| rs1992116 | MMP2 | 4313 | 16 | 54085392 | ‘C/T’ | A | 13.57 | 0.54 |
| rs3886870 | PRKCE | 5581 | 2 | 45954129 | ‘A/G’ | A | 13.56 | 0.41 |
| rs7136446 | IGF1 | 3479 | 12 | 101340982 | ‘C/T’ | G | 13.54 | 0.52 |
| rs12886812 | TTC7B | 145567 | 14 | 90269540 | ‘C/T’ | G | 13.51 | 0.46 |
| rs4360824 | | | 13 | 57707459 | ‘C/T’ | A | 13.50 | 1.80 |
| rs9951631 | DSC1 | 1823 | 18 | 26995905 | ‘C/T’ | A | 13.50 | 3.56 |
| rs1445097 | | | 18 | 48008752 | ‘A/G’ | A | 13.48 | 1.83 |
| rs1441669 | | | 4 | 29265718 | ‘A/G’ | G | 13.46 | 1.80 |
| rs4684011 | | | 3 | 77600301 | ‘C/T’ | A | 13.46 | 0.54 |
| rs2876263 | | | 6 | 135096061 | ‘C/T’ | G | 13.36 | 1.89 |
| rs916351 | | | 20 | 45971417 | ‘A/G’ | A | 13.36 | 1.80 |
| rs564127 | | | 7 | 79540870 | ‘C/T’ | G | 13.24 | 0.38 |
| rs11725230 | FIP1L1 | 81608 | 4 | 54153000 | ‘C/T’ | A | 13.23 | 0.54 |
| rs10053765 | | | 5 | 99021831 | ‘C/T’ | G | 13.21 | 0.52 |
| rs4686599 | | | 3 | 193327814 | ‘C/T’ | G | 13.17 | 0.24 |
| rs4853186 | | | 2 | 76118450 | ‘C/T’ | A | 13.15 | 0.37 |
| rs7791484 | | | 7 | 153594167 | ‘A/G’ | G | 13.14 | 1.78 |
| rs264176 | | | 18 | 10891607 | ‘C/T’ | A | 13.11 | 0.40 |
| rs10774863 | | | 12 | 115353876 | ‘C/T’ | G | 13.09 | 0.54 |
| rs869636 | NRP1 | 8829 | 10 | 33612045 | ‘C/T’ | G | 13.07 | 0.54 |
| rs690901 | | | 5 | 18032928 | ‘C/T’ | G | 13.06 | 2.31 |
| rs4746969 | TYSND1 | 219743 | 10 | 71570195 | ‘G/T’ | C | 13.06 | 2.15 |
| rs7641489 | | | 3 | 45263538 | ‘C/T’ | A | 13.06 | 0.36 |
| rs2591797 | LOC389293 | 389293 | 5 | 62113826 | ‘C/T’ | G | 13.03 | 0.54 |
| rs6676641 | PAPPA2 | 60676 | 1 | 173188880 | ‘G/T’ | C | 13.01 | 1.96 |
| rs911946 | SMOC2 | 64094 | 6 | 168861132 | ‘C/T’ | G | 12.99 | 2.30 |
| rs2048005 | | | 4 | 85180915 | ‘A/C’ | A | 12.99 | 1.78 |
| rs4855460 | | | 3 | 70229264 | ‘G/T’ | A | 12.99 | 0.36 |
| rs761021 | PRKCBP1 | 23613 | 20 | 45327366 | ‘C/T’ | A | 12.98 | 0.40 |
| rs6694274 | LOC149134 | 149134 | 1 | 243280207 | ‘A/G’ | G | 12.96 | 1.97 |
| rs1499306 | | | 13 | 68030461 | ‘C/T’ | A | 12.94 | 0.52 |
| rs4904117 | | | 14 | 83011020 | ‘A/G’ | A | 12.89 | 0.35 |
| rs941763 | LOC440193 | 440193 | 14 | 90890999 | ‘A/G’ | A | 12.85 | 0.33 |
| rs674685 | | | 1 | 191863856 | ‘G/T’ | A | 12.84 | 2.16 |
| rs7648557 | | | 3 | 64658255 | ‘G/T’ | A | 12.83 | 1.80 |
| rs10508468 | FRMD4A | 55691 | 10 | 13958759 | ‘C/T’ | G | 12.82 | 1.84 |
| rs2039183 | | | 9 | 111037906 | ‘A/C’ | A | 12.82 | 0.50 |
| rs6782243 | SLC9A9 | 285195 | 3 | 144891970 | ‘A/G’ | G | 12.77 | 1.77 |
| rs732994 | | | 21 | 41155829 | ‘A/G’ | G | 12.77 | 0.56 |
| rs1454635 | | | 9 | 2780307 | ‘C/T’ | G | 12.75 | 0.56 |
| rs17403547 | | | 2 | 186117324 | ‘G/T’ | C | 12.72 | 4.30 |
| rs2047141 | | | 10 | 31064141 | ‘C/T’ | G | 12.72 | 1.80 |
| rs12146943 | CRYL1 | 51084 | 13 | 19974875 | ‘A/G’ | A | 12.71 | 0.55 |
| rs1324059 | KLF12 | 11278 | 13 | 73241529 | ‘A/G’ | A | 12.62 | 0.54 |
| rs12612914 | ACOXL | 55289 | 2 | 111383160 | ‘A/G’ | A | 12.60 | 2.01 |
| rs12132639 | | | 1 | 61152188 | ‘A/G’ | A | 12.59 | 2.38 |
| rs4709105 | | | 6 | 166586396 | ‘A/G’ | A | 12.56 | 1.77 |
| rs9655857 | | | 7 | 125367411 | ‘C/T’ | G | 12.56 | 1.81 |
| rs1474239 | | | 8 | 20904371 | ‘G/T’ | C | 12.55 | 1.92 |
| rs10518848 | | | 15 | 67966716 | ‘A/G’ | A | 12.52 | 1.77 |
| rs738519 | | | 22 | 35446851 | ‘C/T’ | A | 12.52 | 2.31 |
| rs13417114 | SEC15L2 | 23233 | 2 | 72344951 | ‘A/C’ | A | 12.49 | 0.51 |
| rs717821 | FHIT | 2272 | 3 | 60490818 | ‘C/T’ | G | 12.45 | 2.30 |
| rs8059561 | LOC390683 | 390683 | 16 | 22107225 | ‘C/T’ | G | 12.43 | 2.04 |
| rs1881586 | FRMD4A | 55691 | 10 | 13959631 | ‘C/T’ | G | 12.42 | 1.78 |
| rs1219937 | | | 9 | 25891396 | ‘C/T’ | A | 12.41 | 2.28 |
| rs7238242 | DCC | 1630 | 18 | 48276299 | ‘A/G’ | G | 12.41 | 1.76 |
| rs10862248 | FLJ21963 | 79611 | 12 | 80051990 | ‘G/T’ | C | 12.38 | 0.52 |
| rs9320932 | TRDN | 10345 | 6 | 123786321 | ‘C/T’ | G | 12.36 | 1.81 |
| rs10508274 | | | 10 | 4105602 | ‘A/G’ | G | 12.34 | 0.33 |
| rs7040955 | | | 9 | 102050875 | ‘C/T’ | A | 12.34 | 0.50 |
| rs6585465 | | | 10 | 119610549 | ‘C/T’ | G | 12.33 | 1.76 |
| rs2697668 | | | 8 | 90964181 | ‘A/G’ | G | 12.32 | 1.86 |
| rs257699 | LOC375449 | 375449 | 5 | 66170467 | ‘A/G’ | G | 12.31 | 2.09 |
| rs11939691 | TBC1D14 | 57533 | 4 | 7101749 | ‘C/T’ | A | 12.30 | 1.76 |
| rs1513089 | LOC441062 | 441062 | 5 | 17965365 | ‘A/C’ | C | 12.30 | 2.19 |
| rs2323218 | | | 6 | 166532508 | ‘C/T’ | A | 12.30 | 2.11 |
| rs599140 | | | 1 | 191877580 | ‘C/T’ | A | 12.29 | 2.12 |
| rs2642749 | LOC389293 | 389293 | 5 | 62137231 | ‘A/G’ | G | 12.25 | 1.76 |
| rs10492377 | OVCH1 | 341350 | 12 | 29511714 | ‘C/T’ | G | 12.24 | 2.90 |
| rs7791057 | KIAA1856 | 84629 | 7 | 5205913 | ‘A/G’ | G | 12.22 | 1.82 |
| rs2217228 | | | 12 | 9267916 | ‘C/T’ | A | 12.20 | 1.76 |
| rs8130020 | | | 21 | 41154053 | ‘C/T’ | A | 12.18 | 1.75 |
| rs7003452 | | | 8 | 35140966 | ‘A/G’ | A | 12.18 | 1.75 |
| rs12274588 | | | 11 | 25733789 | ‘A/G’ | A | 12.17 | 0.33 |
| rs2396104 | | | 7 | 108701750 | ‘C/T’ | A | 12.16 | 1.91 |
| rs7930159 | | | 11 | 69133252 | ‘A/G’ | A | 12.13 | 1.79 |
| rs2168908 | | | 4 | 181107283 | ‘C/T’ | G | 12.12 | 2.14 |
| rs1850264 | | | 3 | 201067 | ‘A/G’ | A | 12.11 | 0.57 |
| rs10891888 | | | 11 | 115123297 | ‘G/T’ | A | 12.11 | 1.92 |
| rs12649451 | | | 4 | 172690498 | ‘C/T’ | A | 12.10 | 0.46 |
| rs1389913 | | | 8 | 116231399 | ‘C/T’ | A | 12.10 | 2.49 |
| rs3850970 | | | 12 | 31674144 | ‘C/T’ | G | 12.08 | 0.54 |
| rs1879188 | PTK2B | 2185 | 8 | 27249840 | ‘G/T’ | C | 12.06 | 1.95 |
| rs1879189 | PTK2B | 2185 | 8 | 27254801 | ‘A/G’ | G | 12.06 | 1.95 |
| rs1461272 | NAALADL2 | 254827 | 3 | 176362774 | ‘C/T’ | A | 12.06 | 1.75 |
| rs10492602 | | | 13 | 57737145 | ‘G/T’ | C | 12.06 | 6.85 |
| rs534230 | | | 6 | 143080834 | ‘A/G’ | A | 12.06 | 1.88 |
| rs941924 | MGC4645 | 79446 | 14 | 99962502 | ‘C/T’ | A | 12.04 | 0.56 |
| rs6494794 | | | 15 | 31039019 | ‘C/T’ | G | 12.03 | 2.25 |
| rs2839084 | | | 21 | 46269612 | ‘C/T’ | G | 12.02 | 1.77 |
| rs1838674 | KCNJ3 | 3760 | 2 | 155477049 | ‘A/G’ | G | 12.02 | 2.45 |
| rs12481484 | LOC388790 | 388790 | 20 | 19730668 | ‘A/G’ | A | 12.01 | 0.39 |
| rs6751378 | | | 2 | 78014623 | ‘C/T’ | G | 12.01 | 1.74 |
| rs11771128 | | | 7 | 111669244 | ‘A/G’ | A | 12.00 | 2.06 |
| rs2164349 | | | 3 | 179018450 | ‘A/G’ | G | 11.99 | 0.49 |
| rs911491 | | | 9 | 4232464 | ‘A/G’ | G | 11.99 | 1.80 |
| rs9373941 | C6orf210 | 57107 | 6 | 107758722 | ‘C/T’ | G | 11.98 | 0.53 |
| rs10798460 | PAPPA2 | 60676 | 1 | 173215774 | ‘A/G’ | A | 11.94 | 2.42 |
| rs852766 | DAB1 | 1600 | 1 | 57998529 | ‘A/G’ | G | 11.94 | 1.74 |
| rs11712613 | | | 3 | 67384940 | ‘A/G’ | G | 11.94 | 1.74 |
| rs11655963 | DNAH9 | 1770 | 17 | 11605556 | ‘A/G’ | A | 11.94 | 2.04 |
| rs431474 | | | 19 | 22020632 | ‘C/T’ | A | 11.93 | 1.73 |
| rs6538861 | | | 12 | 97252571 | ‘C/T’ | G | 11.93 | 2.48 |
| rs9285195 | | | 13 | 53568213 | ‘C/T’ | G | 11.93 | 0.53 |
| rs10852366 | | | 16 | 13458041 | ‘C/T’ | A | 11.93 | 0.56 |
| rs1332879 | | | 9 | 80921182 | ‘C/T’ | G | 11.91 | 0.55 |
| rs16931920 | | | 9 | 14533181 | ‘A/C’ | C | 11.91 | 3.11 |
| rs9586037 | | | 13 | 102469005 | ‘G/T’ | A | 11.90 | 0.23 |
| rs4952779 | PRKCE | 5581 | 2 | 45972026 | ‘A/G’ | A | 11.89 | 1.75 |
| rs1880787 | | | 3 | 39792498 | ‘C/T’ | G | 11.87 | 2.38 |
| rs11138526 | | | 9 | 80005011 | ‘G/T’ | A | 11.87 | 2.38 |
| rs7151991 | | | 14 | 31705323 | ‘A/G’ | A | 11.84 | 0.51 |
| rs1507198 | | | 13 | 68131600 | ‘C/T’ | G | 11.81 | 1.74 |
| rs7630843 | | | 3 | 198681 | ‘C/T’ | G | 11.81 | 1.85 |
| rs9307048 | | | 4 | 89506749 | ‘C/T’ | A | 11.81 | 1.85 |
| rs2034875 | | | 9 | 20209315 | ‘C/T’ | A | 11.79 | 0.51 |
| rs4268714 | | | 15 | 29462745 | ‘A/G’ | G | 11.77 | 0.55 |
| rs4131501 | UTRN | 7402 | 6 | 145115486 | ‘C/T’ | G | 11.76 | 0.45 |
| rs7195117 | | | 16 | 13520820 | ‘A/G’ | A | 11.76 | 1.74 |
| rs3892145 | TBC1D14 | 57533 | 4 | 7112156 | ‘C/T’ | G | 11.75 | 1.73 |
| rs10771858 | | | 12 | 31669994 | ‘A/G’ | G | 11.75 | 1.73 |
| rs758896 | FLJ32110 | 219578 | 7 | 88491181 | ‘A/C’ | C | 11.75 | 1.73 |
| rs12372944 | KIAA1199 | 57214 | 15 | 78922504 | ‘C/T’ | A | 11.75 | 1.89 |
| rs2396274 | | | 2 | 226715533 | ‘A/G’ | A | 11.74 | 1.99 |
| rs6800226 | | | 3 | 193336351 | ‘C/T’ | G | 11.74 | 0.26 |
| rs2839081 | | | 21 | 46265743 | ‘C/T’ | G | 11.73 | 1.73 |
| rs7613237 | | | 3 | 185223836 | ‘C/T’ | G | 11.71 | 2.30 |
| rs632912 | | | 18 | 8457707 | ‘A/G’ | A | 11.71 | 4.92 |
| rs13052628 | | | 21 | 41330970 | ‘C/T’ | A | 11.71 | 4.92 |
| rs4559036 | | | 5 | 160209255 | ‘A/C’ | C | 11.71 | 2.41 |
| rs41386 | SEC15L2 | 23233 | 2 | 72341431 | ‘A/G’ | G | 11.69 | 0.50 |
| rs2062206 | | | 2 | 67448093 | ‘G/T’ | C | 11.69 | 0.57 |
| rs6966462 | | | 7 | 153595086 | ‘G/T’ | C | 11.68 | 1.88 |
| rs1978628 | ADAMTS18 | 170692 | 16 | 76002114 | ‘A/G’ | G | 11.67 | 0.48 |
| rs10516437 | | | 4 | 100147223 | ‘A/G’ | G | 11.66 | 0.56 |
| rs3092526 | PRKCBP1 | 23613 | 20 | 45290615 | ‘A/G’ | G | 11.65 | 0.43 |
| rs1594693 | | | 19 | 37309839 | ‘C/T’ | A | 11.64 | 1.78 |
| rs11637685 | KIAA1199 | 57214 | 15 | 78936022 | ‘C/T’ | A | 11.63 | 2.53 |
| rs4941343 | | | 18 | 61347817 | ‘A/G’ | G | 11.61 | 2.23 |
| rs10867485 | | | 9 | 80104890 | ‘A/G’ | G | 11.61 | 2.03 |
| rs4240161 | LSAMP | 4045 | 3 | 117137111 | ‘A/G’ | A | 11.61 | 0.57 |
| rs199253 | | | 6 | 143366889 | ‘C/T’ | A | 11.58 | 1.72 |
| rs2278677 | T | 6862 | 6 | 166546198 | ‘C/T’ | A | 11.57 | 2.05 |
| rs10175158 | PRKCE | 5581 | 2 | 45965912 | ‘C/T’ | A | 11.56 | 1.72 |
| rs10175198 | PRKCE | 5581 | 2 | 45965765 | ‘A/G’ | A | 11.56 | 1.72 |
| rs4953266 | PRKCE | 5581 | 2 | 45965664 | ‘A/G’ | G | 11.56 | 1.72 |
| rs6789298 | ADAMTS9 | 56999 | 3 | 64648855 | ‘C/T’ | A | 11.56 | 2.06 |
| rs1458669 | | | 4 | 181122612 | ‘C/T’ | A | 11.56 | 2.06 |
| rs1993546 | | | 4 | 70020652 | ‘C/T’ | A | 11.54 | 0.51 |
| rs4965671 | | | 15 | 98905466 | ‘A/G’ | G | 11.54 | 2.08 |
| rs9347108 | | | 6 | 166557484 | ‘A/G’ | A | 11.53 | 2.14 |
| rs895491 | GLI2 | 2736 | 2 | 121295057 | ‘A/G’ | G | 11.52 | 0.57 |
| rs12652513 | | | 5 | 99057352 | ‘A/G’ | G | 11.51 | 0.33 |
| rs10489477 | PAPPA2 | 60676 | 1 | 173294265 | ‘C/T’ | A | 11.49 | 2.26 |
| rs131003 | | | 22 | 47485954 | ‘G/T’ | A | 11.49 | 0.41 |
| rs12770610 | CDH23 | 64072 | 10 | 72876293 | ‘C/T’ | G | 11.48 | 1.72 |
| rs4284884 | GPR39 | 2863 | 2 | 133087118 | ‘A/G’ | G | 11.48 | 0.56 |
| rs2561642 | | | 5 | 18135523 | ‘C/T’ | A | 11.48 | 1.73 |
| rs1503994 | | | 4 | 21289743 | ‘C/T’ | A | 11.47 | 1.92 |
| rs10498134 | PAX3 | 5077 | 2 | 222930485 | ‘C/T’ | G | 11.47 | 1.72 |
| rs2135845 | | | 17 | 6787797 | ‘C/T’ | G | 11.47 | 1.72 |
| rs575291 | LOC441822 | 441822 | 18 | 63653262 | ‘C/T’ | A | 11.46 | 2.44 |
| rs7775153 | | | 6 | 137827982 | ‘C/T’ | A | 11.45 | 0.53 |
| rs4689558 | TBC1D14 | 57533 | 4 | 7038538 | ‘A/G’ | G | 11.44 | 1.72 |
| rs17661314 | FHIT | 2272 | 3 | 60462114 | ‘A/C’ | C | 11.44 | 2.00 |
| rs421239 | | | 5 | 172955019 | ‘A/G’ | A | 11.44 | 0.57 |
| rs997448 | | | 3 | 64935253 | ‘A/G’ | G | 11.42 | 2.51 |
| rs706411 | DAB1 | 1600 | 1 | 58004642 | ‘A/G’ | A | 11.42 | 0.58 |
| rs13094898 | RPP14 | 11102 | 3 | 58265183 | ‘A/G’ | A | 11.41 | 0.36 |
| rs4749567 | | | 10 | 30885044 | ‘A/G’ | A | 11.40 | 1.76 |
| rs9958350 | | | 18 | 69134108 | ‘A/G’ | G | 11.38 | 1.79 |
| rs9618567 | HIRA | 7290 | 22 | 17788260 | ‘C/T’ | A | 11.38 | 3.79 |
| rs243842 | MMP2 | 4313 | 16 | 54084923 | ‘C/T’ | G | 11.36 | 1.71 |
| rs1470964 | | | 8 | 115871388 | ‘A/G’ | A | 11.34 | 1.73 |
| rs421548 | SEMA5A | 9037 | 5 | 9561979 | ‘C/T’ | A | 11.33 | 2.46 |
| rs10514626 | | | 2 | 19419621 | ‘A/G’ | A | 11.33 | 2.46 |
| rs4246958 | | | 11 | 68968411 | ‘C/T’ | G | 11.31 | 1.71 |
| rs7984974 | CLDN10 | 9071 | 13 | 94926858 | ‘A/G’ | G | 11.31 | 1.71 |
| rs2303933 | SLC4A2 | 6522 | 7 | 150204447 | ‘A/G’ | A | 11.30 | 1.79 |
| rs1109793 | MGC52010 | 91582 | 22 | 38254446 | ‘C/T’ | G | 11.30 | 1.79 |
| rs11847484 | | | 14 | 95515268 | ‘A/G’ | G | 11.30 | 0.39 |
| rs287871 | ARID1B | 57492 | 6 | 157293419 | ‘C/T’ | G | 11.29 | 1.71 |
| rs6494940 | | | 15 | 69592172 | ‘A/G’ | G | 11.28 | 2.22 |
| rs246565 | | | 5 | 71845003 | ‘A/C’ | A | 11.27 | 0.48 |
| rs246580 | | | 5 | 71851370 | ‘C/T’ | A | 11.27 | 0.48 |
| rs1462404 | | | 5 | 99102291 | ‘C/T’ | G | 11.22 | 0.38 |
| rs7652210 | CKLFSF8 | 152189 | 3 | 32364379 | ‘C/T’ | A | 11.22 | 1.85 |
| rs7283829 | | | 21 | 46252267 | ‘A/G’ | A | 11.21 | 1.74 |
| rs11775958 | PTK2B | 2185 | 8 | 27325801 | ‘G/T’ | C | 11.19 | 1.93 |
| rs171508 | | | 8 | 54081989 | ‘C/T’ | G | 11.19 | 1.77 |
| rs4752130 | | | 10 | 119678690 | ‘C/T’ | G | 11.18 | 0.57 |
| rs7747120 | | | 6 | 52706541 | ‘A/G’ | A | 11.17 | 5.44 |
| rs4763736 | | | 12 | 11964019 | ‘C/T’ | G | 11.17 | 1.73 |
| rs4921165 | | | 5 | 160212958 | ‘G/T’ | C | 11.16 | 2.21 |
| rs1399130 | | | 4 | 28257074 | ‘A/G’ | G | 11.14 | 1.70 |
| rs11225285 | | | 11 | 101883403 | ‘C/T’ | A | 11.14 | 2.72 |
| rs420444 | OSMR | 9180 | 5 | 38893123 | ‘A/C’ | C | 11.14 | 1.90 |
| rs908720 | | | 2 | 226720486 | ‘A/G’ | G | 11.11 | 1.94 |
| rs7554508 | MART2 | 55733 | 1 | 207025945 | ‘C/T’ | A | 11.11 | 1.71 |
| rs1005516 | LOC441284 | 441284 | 7 | 140267483 | ‘C/T’ | A | 11.11 | 1.70 |
| rs9346693 | | | 6 | 168566847 | ‘A/C’ | C | 11.10 | 1.71 |
| rs3890755 | MEIS1 | 4211 | 2 | 66656254 | ‘C/T’ | G | 11.07 | 1.70 |
| rs6540951 | | | 1 | 10813991 | ‘A/G’ | A | 11.07 | 0.42 |
| rs6940007 | SLC17A2 | 10246 | 6 | 26039936 | ‘A/C’ | C | 11.06 | 4.31 |
| rs878554 | | | 14 | 93675932 | ‘C/T’ | A | 11.06 | 0.52 |
| rs11743832 | | | 5 | 51230147 | ‘C/T’ | G | 11.06 | 0.57 |
| rs11630449 | | | 15 | 29402033 | ‘C/T’ | G | 11.05 | 0.50 |
| rs8074227 | | | 17 | 12484440 | ‘G/T’ | C | 11.01 | 1.84 |
| rs1223016 | | | 4 | 172618238 | ‘A/G’ | G | 11.00 | 0.58 |
| rs4665830 | LOC150946 | 150946 | 2 | 26310865 | ‘A/G’ | G | 11.00 | 0.58 |
| rs4446382 | | | 4 | 135263866 | ‘A/G’ | A | 10.98 | 0.50 |
| rs11780975 | | | 8 | 103536662 | ‘A/C’ | A | 10.98 | 0.12 |
| rs10501022 | | | 11 | 25712397 | ‘C/T’ | G | 10.98 | 0.33 |
| rs6997351 | FLJ32370 | 137695 | 8 | 56814300 | ‘A/G’ | G | 10.98 | 0.57 |
| rs7027886 | | | 9 | 2788358 | ‘A/C’ | A | 10.97 | 0.59 |
| rs11647932 | SIAT4B | 6483 | 16 | 69022319 | ‘C/T’ | A | 10.95 | 2.27 |
| rs12325419 | | | 16 | 68926410 | ‘A/G’ | A | 10.95 | 2.27 |
| rs10503533 | | | 8 | 15030038 | ‘A/G’ | G | 10.95 | 1.82 |
| rs7151110 | TTC7B | 145567 | 14 | 90222235 | ‘C/T’ | G | 10.95 | 0.49 |
| rs7603494 | | | 2 | 47815747 | ‘C/T’ | G | 10.95 | 1.70 |
| rs6937983 | C6orf210 | 57107 | 6 | 107773941 | ‘A/G’ | A | 10.95 | 1.70 |
| rs7981816 | | | 13 | 71540844 | ‘A/G’ | G | 10.93 | 2.99 |
| rs6769747 | CHL1 | 10752 | 3 | 219949 | ‘A/G’ | G | 10.91 | 2.50 |
| rs4864767 | LNX | 84708 | 4 | 54208064 | ‘A/G’ | A | 10.91 | 0.57 |
| rs1517927 | | | 3 | 64977356 | ‘A/G’ | A | 10.89 | 1.99 |
| rs9679386 | CAPN13 | 92291 | 2 | 30864638 | ‘C/T’ | A | 10.89 | 0.57 |
| rs32790 | | | 5 | 35466853 | ‘A/G’ | G | 10.88 | 0.30 |
| rs9350132 | | | 6 | 19498105 | ‘C/T’ | A | 10.88 | 2.22 |
| rs2178531 | | | 12 | 11512616 | ‘C/T’ | G | 10.87 | 0.34 |
| rs3733787 | | | 5 | 5131313 | ‘A/G’ | A | 10.87 | 1.72 |
| rs2279307 | | | 12 | 83190484 | ‘C/T’ | G | 10.86 | 0.45 |
| rs6561970 | | | 13 | 58021020 | ‘A/G’ | G | 10.86 | 0.46 |
| rs1384394 | | | 2 | 213664375 | ‘C/T’ | A | 10.86 | 2.64 |
| rs12653539 | | | 5 | 98501952 | ‘A/C’ | C | 10.86 | 0.07 |
| rs10483395 | | | 14 | 31708664 | ‘A/G’ | A | 10.85 | 2.01 |
| rs7872903 | | | 9 | 133513846 | ‘C/T’ | G | 10.85 | 0.47 |
| rs1809366 | | | 15 | 31051600 | ‘A/G’ | G | 10.84 | 0.52 |
| rs899466 | | | 15 | 31051493 | ‘A/G’ | G | 10.84 | 0.52 |
| rs41420 | | | 2 | 72313909 | ‘C/T’ | A | 10.84 | 0.52 |
| rs13078878 | | | 3 | 64980991 | ‘C/T’ | G | 10.83 | 2.17 |
| rs2593430 | CAPN13 | 92291 | 2 | 30863635 | ‘C/T’ | G | 10.83 | 0.59 |
| rs2233696 | PLP1 | 5354 | X | 102846545 | ‘C/T’ | G | 23.15 | 2.22 |
| rs5909473 | | | X | 18179407 | ‘A/G’ | G | 18.82 | 2.78 |
| rs2765386 | DMD | 1756 | X | 32825545 | ‘C/T’ | G | 18.56 | 0.39 |
| rs2366517 | | | X | 136785639 | ‘C/T’ | G | 18.22 | 2.01 |
| rs3007187 | | | X | 112965574 | ‘A/G’ | A | 17.22 | 0.47 |
| rs12012576 | | | X | 21572835 | ‘A/G’ | G | 17.10 | 0.48 |
| rs2366513 | | | X | 136776197 | ‘A/G’ | G | 16.73 | 1.95 |
| rs10521502 | | | X | 102861176 | ‘A/G’ | A | 16.57 | 2.16 |
| rs5978303 | | | X | 9008007 | ‘A/G’ | G | 16.37 | 1.97 |
| rs5910338 | | | X | 117242500 | ‘C/T’ | G | 16.23 | 3.30 |
| rs5910340 | | | X | 117247923 | ‘C/T’ | G | 16.23 | 3.30 |
| rs2765385 | DMD | 1756 | X | 32823299 | ‘A/G’ | A | 15.67 | 0.41 |
| rs4827759 | | | X | 143796824 | ‘A/G’ | A | 15.56 | 2.17 |
| rs616364 | GRIA3 | 2892 | X | 122315581 | ‘A/G’ | G | 15.53 | 2.10 |
| rs12558663 | | | X | 98465719 | ‘G/T’ | A | 15.50 | 19.07 |
| rs6418743 | | | X | 21541534 | ‘C/T’ | G | 15.24 | 0.53 |
| rs4503212 | RGN | 9104 | X | 46697712 | ‘A/G’ | G | 14.75 | 0.47 |
| rs2886700 | | | X | 136747068 | ‘C/T’ | G | 14.56 | 1.85 |
| rs2031556 | DMD | 1756 | X | 32837563 | ‘C/T’ | A | 14.52 | 0.50 |
| rs2814862 | DMD | 1756 | X | 32821726 | ‘A/C’ | A | 14.39 | 0.44 |
| rs5987579 | | | X | 102817760 | ‘A/C’ | A | 14.39 | 1.87 |
| rs5937060 | | | X | 70024726 | ‘C/T’ | G | 14.15 | 1.85 |
| rs12840573 | TNRC11 | 9968 | X | 70121139 | ‘A/G’ | A | 13.85 | 3.77 |
| rs6527253 | DMD | 1756 | X | 32838651 | ‘C/T’ | G | 13.67 | 0.50 |
| rs2313032 | | | X | 25506084 | ‘A/G’ | A | 13.47 | 1.83 |
| rs3012658 | HDAC8 | 55869 | X | 71349766 | ‘C/T’ | G | 13.26 | 0.06 |
| rs7880245 | | | X | 5238573 | ‘C/T’ | A | 13.17 | 1.87 |
| rs4460510 | | | X | 145845745 | ‘G/T’ | A | 13.15 | 1.81 |
| rs7058356 | | | X | 5252012 | ‘A/G’ | G | 13.12 | 1.99 |
| rs3922743 | | | X | 13910650 | ‘A/G’ | G | 12.99 | 1.78 |
| rs2813809 | | | X | 146355970 | ‘A/G’ | A | 12.94 | 0.53 |
| rs2622953 | | | X | 121245542 | ‘G/T’ | A | 12.81 | 0.15 |
| rs1936648 | LOC392456 | 392456 | X | 45905900 | ‘A/G’ | G | 12.77 | 2.51 |
| rs5951469 | | | X | 21608265 | ‘C/T’ | A | 12.70 | 0.56 |
| rs3092921 | TNFSF5 | 959 | X | 135468520 | ‘C/T’ | A | 12.63 | 0.23 |
| rs6627187 | | | X | 150623478 | ‘C/T’ | G | 12.52 | 1.80 |
| rs2574054 | PCDH11X | 27328 | X | 91385669 | ‘A/G’ | A | 12.50 | 1.76 |
| rs7878576 | | | X | 21596302 | ‘A/G’ | A | 12.36 | 1.75 |
| rs5905269 | | | X | 115300034 | ‘A/C’ | A | 12.26 | 0.54 |
| rs1573036 | | | X | 109626213 | ‘A/G’ | A | 12.16 | 0.56 |
| rs5942651 | | | X | 109633767 | ‘A/G’ | G | 12.16 | 0.56 |
| rs475827 | PLP1 | 5354 | X | 102836217 | ‘C/T’ | A | 12.13 | 0.50 |
| rs845188 | | | X | 140102408 | ‘A/G’ | G | 12.06 | 1.75 |
| rs986342 | IL1RAPL1 | 11141 | X | 28601486 | ‘G/T’ | A | 11.94 | 2.60 |
| rs2240584 | DXS1283E | 8228 | X | 7693630 | ‘A/G’ | A | 11.84 | 1.76 |
| rs4585878 | | | X | 44967214 | ‘A/G’ | G | 11.79 | 0.55 |
| rs5931268 | | | X | 136791119 | ‘G/T’ | A | 11.72 | 0.56 |
| rs6527813 | | | X | 13298834 | ‘C/T’ | A | 11.70 | 0.48 |
| rs5969826 | | | X | 150622572 | ‘A/G’ | G | 11.69 | 1.76 |
| rs2761647 | | | X | 95078803 | ‘A/C’ | C | 11.45 | 0.31 |
| rs4911823 | | | X | 114478198 | ‘C/T’ | G | 11.45 | 1.76 |
| rs1401413 | | | X | 113553486 | ‘A/G’ | A | 11.18 | 0.24 |
| rs5945988 | | | X | 113542162 | ‘A/G’ | A | 11.18 | 0.24 |
| rs5934569 | | | X | 9088619 | ‘G/T’ | A | 11.18 | 0.48 |
| rs845127 | | | X | 7635061 | ‘A/G’ | A | 11.15 | 0.35 |
| rs2071211 | MBTPS2 | 51360 | X | 21629701 | ‘A/G’ | A | 10.97 | 0.59 |
| rs1560517 | | | X | 13297246 | ‘A/G’ | A | 10.96 | 0.54 |
| rs4829455 | AMOT | 154796 | X | 111870035 | ‘A/C’ | A | 10.95 | 2.27 |
| rs10465305 | | | X | 87621994 | ‘A/G’ | A | 10.90 | 0.51 |
| rs4828697 | | | X | 151337295 | ‘C/T’ | G | 10.85 | 2.20 |

dbSNP_rs_ID: SNP identification number in NCBI dbSNP database
Gene_locus: Gene locus and gene id as reported by NCBI dbSNP database build 126
Sequence_ID: Sequence identification number
Position: Basepair Position, SNP physical position according to NCBI Human Genome Build 36.1
Variants: Alternate SNP alleles or their complementary nucleotides in the position indicated by dbSNP RS ID and basepair position
Minor Allele: SNP allele or its complementary nucleotide that is less common in the control population.
Allele_X2: Chi-squared test based on allele frequencies
Odds ratio: Calculated for the minor allele.
Gene_content: Genes positioned within 100 Kbp up and downstream from the physical position of the SNPs based on NCBI Human Genome Build 36.1

TABLE 3
Haplotype genomic regions with the strongest association with HT in the
haplotype sharing analysis (HaploRec + HPM) with 8 SNPs. The analysis
is based on 140 HT cases and 182 healthy controls from East Finland.

| | Gene locus | | | | | |
|---|---|---|---|---|---|---|
| | and | | | | | |
| dbSNP rs ID | Gene ID | | Chromosome | Position | Variats | P value |
| rs6676641 | PAPPA2 | 60676 | 1 | 173188880 | ‘G/T’ | 0.0003 |
| rs12084712 | PAPPA2 | 60676 | 1 | 173214758 | ‘A/G’ | <0.0001 |
| rs10798460 | PAPPA2 | 60676 | 1 | 173215774 | ‘A/G’ | <0.0001 |
| rs11801416 | PAPPA2 | 60676 | 1 | 173223334 | ‘C/T’ | <0.0001 |
| rs2206509 | PAPPA2 | 60676 | 1 | 173238947 | ‘C/T’ | 0.0004 |
| rs2901091 | | | 2 | 72294583 | ‘C/T’ | 0.0006 |
| rs975612 | | | 2 | 72300989 | ‘A/C’ | 0.0002 |
| rs41420 | | | 2 | 72313909 | ‘C/T’ | 0.0001 |
| rs41419 | SEC15L2 | 23233 | 2 | 72314465 | ‘A/G’ | 0.0001 |
| rs41402 | SEC15L2 | 23233 | 2 | 72330710 | ‘A/C’ | 0.0001 |
| rs41386 | SEC15L2 | 23233 | 2 | 72341431 | ‘A/G’ | <0.0001 |
| rs194235 | SEC15L2 | 23233 | 2 | 72342517 | ‘A/G’ | <0.0001 |
| rs13417114 | SEC15L2 | 23233 | 2 | 72344951 | ‘A/C’ | 0.0003 |
| rs7565922 | SEC15L2 | 23233 | 2 | 72353375 | ‘A/G’ | 0.0006 |
| rs11897719 | SLC16A14 | 151473 | 2 | 230754953 | ‘C/T’ | 0.0002 |
| rs12475755 | | | 2 | 230761259 | ‘A/G’ | <0.0001 |
| rs4613264 | | | 2 | 230768956 | ‘A/G’ | <0.0001 |
| rs12162384 | | | 2 | 230785037 | ‘A/C’ | 0.0001 |
| rs7599215 | | | 2 | 230786451 | ‘G/T’ | 0.0001 |
| rs12694836 | | | 2 | 230813653 | ‘A/G’ | 0.0001 |
| rs6436908 | | | 2 | 230820952 | ‘C/T’ | 0.0002 |
| rs6764952 | | | 3 | 178984873 | ‘A/C’ | 0.0006 |
| rs4857745 | | | 3 | 178988464 | ‘C/T’ | 0.0001 |
| rs1984961 | | | 3 | 178992805 | ‘A/G’ | <0.0001 |
| rs1004448 | | | 3 | 178992971 | ‘C/T’ | 0.0001 |
| rs2863060 | | | 3 | 178997416 | ‘A/G’ | 0.0001 |
| rs4857746 | | | 3 | 179003470 | ‘C/T’ | 0.0002 |
| rs7651231 | | | 3 | 179007870 | ‘A/G’ | 0.0001 |
| rs9861373 | | | 3 | 179010065 | ‘A/G’ | <0.0001 |
| rs4857747 | | | 3 | 179014549 | ‘C/T’ | <0.0001 |
| rs2164349 | | | 3 | 179018450 | ‘A/G’ | <0.0001 |
| rs7641262 | | | 3 | 179025554 | ‘C/T’ | <0.0001 |
| rs1561030 | | | 3 | 179034481 | ‘A/G’ | <0.0001 |
| rs4857750 | | | 3 | 179047846 | ‘A/G’ | <0.0001 |
| rs10936959 | | | 3 | 179073776 | ‘A/G’ | 0.0001 |
| rs7612209 | | | 3 | 179079691 | ‘A/G’ | 0.001 |
| rs6534907 | | | 4 | 134100931 | ‘C/T’ | 0.0009 |
| rs1868251 | | | 4 | 134112404 | ‘A/G’ | 0.0001 |
| rs10518621 | | | 4 | 134122347 | ‘C/T’ | <0.0001 |
| rs10518622 | | | 4 | 134122952 | ‘A/G’ | 0.0006 |
| rs10486903 | FLJ32110 | 219578 | 7 | 88427903 | ‘C/T’ | 0.0008 |
| rs10486904 | FLJ32110 | 219578 | 7 | 88429704 | ‘G/T’ | 0.0006 |
| rs10486905 | FLJ32110 | 219578 | 7 | 88435260 | ‘C/T’ | 0.0001 |
| rs2189052 | FLJ32110 | 219578 | 7 | 88438850 | ‘C/T’ | 0.0002 |
| rs720142 | FLJ32110 | 219578 | 7 | 88444330 | ‘A/G’ | <0.0001 |
| rs2214339 | FLJ32110 | 219578 | 7 | 88457940 | ‘A/G’ | <0.0001 |
| rs7799723 | FLJ32110 | 219578 | 7 | 88458675 | ‘C/T’ | 0.0003 |
| rs7844565 | COL22A1 | 169044 | 8 | 139755924 | ‘C/T’ | 0.0005 |
| rs7839680 | COL22A1 | 169044 | 8 | 139762863 | ‘A/G’ | 0.0002 |
| rs4909443 | COL22A1 | 169044 | 8 | 139770093 | ‘A/G’ | 0.0001 |
| rs4909444 | COL22A1 | 169044 | 8 | 139770391 | ‘G/T’ | <0.0001 |
| rs7835385 | COL22A1 | 169044 | 8 | 139778987 | ‘G/T’ | <0.0001 |
| rs4243905 | COL22A1 | 169044 | 8 | 139783913 | ‘A/G’ | <0.0001 |
| rs4074052 | COL22A1 | 169044 | 8 | 139785008 | ‘C/T’ | <0.0001 |
| rs11166837 | COL22A1 | 169044 | 8 | 139791315 | ‘C/T’ | <0.0001 |
| rs12681358 | COL22A1 | 169044 | 8 | 139794900 | ‘C/T’ | 0.0001 |
| rs9324493 | COL22A1 | 169044 | 8 | 139797163 | ‘A/G’ | 0.0008 |
| rs10509845 | | | 10 | 109510824 | ‘C/T’ | 0.0005 |
| rs2418977 | | | 10 | 109511578 | ‘G/T’ | 0.0004 |
| rs7912221 | | | 10 | 109515057 | ‘A/C’ | 0.0002 |
| rs2900778 | | | 10 | 109517065 | ‘A/C’ | 0.0002 |
| rs2418976 | | | 10 | 109529198 | ‘A/C’ | <0.0001 |
| rs4431961 | | | 10 | 109529269 | ‘C/T’ | <0.0001 |
| rs2900784 | | | 10 | 109539457 | ‘A/C’ | 0.0002 |
| rs1025888 | CNTN5 | 53942 | 11 | 99252539 | ‘A/G’ | 0.0002 |
| rs7931411 | CNTN5 | 53942 | 11 | 99259555 | ‘A/G’ | 0.0001 |
| rs10501927 | CNTN5 | 53942 | 11 | 99262939 | ‘G/T’ | <0.0001 |
| rs1971156 | CNTN5 | 53942 | 11 | 99263024 | ‘C/T’ | <0.0001 |
| rs1453590 | CNTN5 | 53942 | 11 | 99271543 | ‘A/C’ | 0.0003 |
| rs2769556 | | | 13 | 67976305 | ‘A/G’ | 0.0005 |
| rs9541407 | | | 13 | 67978683 | ‘G/T’ | 0.0003 |
| rs1240891 | | | 13 | 67980008 | ‘A/C’ | 0.0002 |
| rs904510 | | | 13 | 67986542 | ‘C/T’ | 0.0002 |
| rs7997100 | | | 13 | 67988075 | ‘C/T’ | 0.0004 |
| rs12184778 | | | 13 | 68006426 | ‘A/G’ | 0.0002 |
| rs976211 | | | 13 | 68016099 | ‘A/C’ | 0.0003 |
| rs17557736 | | | 13 | 68027160 | ‘A/G’ | 0.0002 |
| rs9571951 | | | 13 | 68030249 | ‘C/T’ | 0.0002 |
| rs1499306 | | | 13 | 68030461 | ‘C/T’ | <0.0001 |
| rs2248276 | | | 13 | 68033135 | ‘A/G’ | 0.0001 |
| rs9541444 | | | 13 | 68045251 | ‘C/T’ | 0.0002 |
| rs287312 | | | 13 | 68069581 | ‘A/G’ | 0.0004 |
| rs287320 | | | 13 | 68073566 | ‘G/T’ | 0.0004 |
| rs287327 | | | 13 | 68076868 | ‘A/G’ | 0.0003 |
| rs10507750 | | | 13 | 68079566 | ‘A/G’ | 0.0002 |
| rs287409 | | | 13 | 68103919 | ‘A/G’ | 0.0002 |
| rs9541476 | | | 13 | 68110347 | ‘C/T’ | 0.0003 |
| rs7151991 | | | 14 | 31705323 | ‘A/G’ | 0.0002 |
| rs10483395 | | | 14 | 31708664 | ‘A/G’ | 0.0001 |
| rs17098539 | | | 14 | 31711014 | ‘G/T’ | <0.0001 |
| rs4476082 | | | 14 | 31722876 | ‘A/C’ | 0.0003 |
| rs2147829 | TTC7B | 145567 | 14 | 90233622 | ‘C/T’ | 0.0004 |
| rs3814841 | TTC7B | 145567 | 14 | 90234219 | ‘C/T’ | 0.0003 |
| rs1742098 | TTC7B | 145567 | 14 | 90238170 | ‘C/T’ | 0.0002 |
| rs1749704 | TTC7B | 145567 | 14 | 90239107 | ‘G/T’ | <0.0001 |
| rs1535321 | TTC7B | 145567 | 14 | 90240579 | ‘C/T’ | <0.0001 |
| rs1749718 | TTC7B | 145567 | 14 | 90253080 | ‘C/T’ | <0.0001 |
| rs1742083 | TTC7B | 145567 | 14 | 90256423 | ‘C/T’ | <0.0001 |
| rs8018904 | TTC7B | 145567 | 14 | 90259730 | ‘G/T’ | 0.0001 |
| rs12886812 | TTC7B | 145567 | 14 | 90269540 | ‘C/T’ | 0.0001 |
| rs730043 | TTC7B | 145567 | 14 | 90279368 | ‘G/T’ | 0.0005 |
| rs7158495 | TTC7B | 145567 | 14 | 90281628 | ‘C/T’ | 0.0005 |
| rs1535188 | C14orf49 | 161176 | 14 | 94997166 | ‘C/T’ | 0.0006 |
| rs2012192 | C14orf49 | 161176 | 14 | 94998087 | ‘A/G’ | <0.0001 |
| rs3783290 | C14orf49 | 161176 | 14 | 94999953 | ‘G/T’ | 0.0006 |
| rs9302671 | MMP2 | 4313 | 16 | 54079226 | ‘G/T’ | 0.001 |
| rs243842 | MMP2 | 4313 | 16 | 54084923 | ‘C/T’ | 0.0001 |
| rs1992116 | MMP2 | 4313 | 16 | 54085392 | ‘C/T’ | <0.0001 |
| rs243840 | MMP2 | 4313 | 16 | 54085660 | ‘A/G’ | 0.0001 |
| rs243834 | MMP2 | 4313 | 16 | 54094188 | ‘A/G’ | 0.0007 |
| rs6142710 | | | 20 | 60091799 | ‘A/G’ | <0.0001 |
| rs6142711 | | | 20 | 60095481 | ‘A/G’ | <0.0001 |
| rs6142946 | | | 20 | 60106460 | ‘G/T’ | <0.0001 |
| rs2038687 | C20orf40 | 149986 | 20 | 60140435 | ‘C/T’ | 0.0001 |
| rs2057169 | PSMA7 | 5688 | 20 | 60145679 | ‘C/T’ | 0.001 |
| rs7892324 | | | X | 6529033 | ‘C/T’ | 0.0005 |
| rs6638625 | | | X | 6562245 | ‘A/G’ | 0.0002 |
| rs6639674 | | | X | 6568014 | ‘A/G’ | <0.0001 |
| rs968021 | | | X | 18136978 | ‘G/T’ | 0.0003 |
| rs5955619 | | | X | 18137779 | ‘A/G’ | 0.0001 |
| rs5909473 | | | X | 18179407 | ‘A/G’ | <0.0001 |
| rs5955621 | CDKL5 | 6792 | X | 18209165 | ‘A/G’ | 0.0003 |
| rs2061249 | DMD | 1756 | X | 32077365 | ‘C/T’ | 0.0004 |
| rs331322 | DMD | 1756 | X | 32077588 | ‘A/G’ | <0.0001 |
| rs331321 | DMD | 1756 | X | 32078204 | ‘A/G’ | <0.0001 |
| rs331320 | DMD | 1756 | X | 32078628 | ‘C/T’ | <0.0001 |
| rs5927962 | DMD | 1756 | X | 32081017 | ‘C/T’ | 0.0001 |
| rs331318 | DMD | 1756 | X | 32084123 | ‘C/T’ | 0.0004 |
| rs483812 | | | X | 102802880 | ‘C/T’ | <0.0001 |
| rs568707 | | | X | 102813341 | ‘C/T’ | <0.0001 |
| rs5987579 | | | X | 102817760 | ‘A/C’ | <0.0001 |
| rs554412 | | | X | 102821525 | ‘C/T’ | <0.0001 |
| rs475827 | PLP1 | 5354 | X | 102836217 | ‘C/T’ | <0.0001 |
| rs521895 | PLP1 | 5354 | X | 102842557 | ‘A/G’ | <0.0001 |
| rs2233696 | PLP1 | 5354 | X | 102846545 | ‘C/T’ | <0.0001 |
| rs2294152 | PLP1 | 5354 | X | 102849879 | ‘G/T’ | <0.0001 |
| rs10521502 | | | X | 102861176 | ‘A/G’ | 0.0001 |
| rs5942641 | | | X | 109549379 | ‘A/G’ | 0.0007 |
| rs1573036 | | | X | 109626213 | ‘A/G’ | <0.0001 |
| rs5942651 | | | X | 109633767 | ‘A/G’ | <0.0001 |
| rs197023 | CHRDL1 | 91851 | X | 109774532 | ‘C/T’ | 0.0001 |
| rs12689346 | CHRDL1 | 91851 | X | 109810107 | ‘C/T’ | 0.0004 |
| rs5985312 | | | X | 110000370 | ‘A/G’ | 0.0007 |
| rs5910156 | | | X | 116445879 | ‘C/T’ | 0.0004 |
| rs5912022 | | | X | 116457000 | ‘C/T’ | <0.0001 |
| rs6646995 | | | X | 116468033 | ‘G/T’ | <0.0001 |
| rs5958727 | | | X | 116515613 | ‘C/T’ | 0.0004 |
| rs742217 | | | X | 136286956 | ‘A/G’ | 0.0003 |
| rs2859257 | | | X | 136309117 | ‘A/G’ | <0.0001 |
| rs6635446 | | | X | 136326022 | ‘C/T’ | 0.0001 |
| rs6635777 | | | X | 137993914 | ‘G/T’ | 0.0002 |
| rs5974805 | | | X | 137998316 | ‘A/G’ | 0.0001 |
| rs12558495 | | | X | 138027981 | ‘C/T’ | <0.0001 |
| rs5974808 | | | X | 138035117 | ‘A/G’ | 0.0002 |
| rs2813808 | | | X | 146353663 | ‘A/G’ | 0.0006 |
| rs5951805 | | | X | 146353775 | ‘C/T’ | <0.0001 |
| rs2813809 | | | X | 146355970 | ‘A/G’ | <0.0001 |
| rs7890402 | | | X | 146360070 | ‘A/G’ | 0.0001 |
| rs742581 | | | X | 149201394 | ‘C/T’ | 0.0005 |
| rs614511 | | | X | 149208402 | ‘A/G’ | 0.0001 |
| rs5925535 | | | X | 149210151 | ‘C/T’ | <0.0001 |
| rs693913 | | | X | 149212422 | ‘C/T’ | <0.0001 |
| rs10776290 | | | X | 149224831 | ‘A/C’ | 0.0001 |
| rs5924915 | | | X | 149231128 | ‘A/G’ | 0.0002 |
| rs2814855 | DMD | 1756 | X | 32813402 | ‘C/T’ | 0.0008 |
| rs2814862 | DMD | 1756 | X | 32821726 | ‘A/C’ | 0.0001 |
| rs2765385 | DMD | 1756 | X | 32823299 | ‘A/G’ | 0.0002 |
| rs982767 | DMD | 1756 | X | 32824337 | ‘C/T’ | 0.0004 |
| rs2765386 | DMD | 1756 | X | 32825545 | ‘C/T’ | 0.0003 |
| rs2031554 | DMD | 1756 | X | 32833444 | ‘C/T’ | 0.0002 |
| rs2031556 | DMD | 1756 | X | 32837563 | ‘C/T’ | 0.0001 |
| rs6527253 | DMD | 1756 | X | 32838651 | ‘C/T’ | 0.0002 |
| rs6624142 | LOC441496 | 441496 | X | 64188190 | ‘C/T’ | 0.0004 |
| rs10465337 | | | X | 64806428 | ‘A/G’ | 0.0001 |
| rs5918959 | | | X | 64810327 | ‘C/T’ | 0.0001 |
| rs2366551 | | | X | 136746208 | ‘C/T’ | 0.0005 |
| rs2886700 | | | X | 136747068 | ‘C/T’ | 0.0002 |
| rs2366513 | | | X | 136776197 | ‘A/G’ | 0.0001 |
| rs2366517 | | | X | 136785639 | ‘C/T’ | 0.0001 |
| rs5931268 | | | X | 136791119 | ‘G/T’ | 0.0002 |
| rs5931272 | | | X | 136803377 | ‘A/G’ | 0.001 |
| rs1551504 | | | X | 136807832 | ‘A/C’ | 0.001 |
| rs1560303 | | | X | 136813658 | ‘A/G’ | 0.0007 |
| rs6528506 | | | X | 136827208 | ‘C/T’ | 0.0005 |
| rs5929877 | | | X | 136854327 | ‘A/G’ | 0.0004 |
| rs11795896 | | | X | 136865816 | ‘C/T’ | 0.0005 |
| rs12556519 | | | X | 136867643 | ‘G/T’ | 0.0004 |
| rs6635565 | | | X | 136889896 | ‘C/T’ | 0.0008 |
| rs5929883 | | | X | 136902901 | ‘C/T’ | 0.0009 |
| rs585602 | | | X | 136922918 | ‘A/G’ | 0.0009 |
| rs5936254 | | | X | 148064327 | ‘C/T’ | 0.0006 |
| rs764908 | | | X | 148082914 | ‘C/T’ | 0.0001 |
| rs12859656 | | | X | 148095061 | ‘A/G’ | 0.0001 |
| rs1882731 | | | X | 148111861 | ‘C/T’ | 0.0003 |
| rs9698926 | | | X | 149093381 | ‘C/T’ | 0.0001 |
| rs4953260 | PRKCE | 5581 | 2 | 45945370 | ‘C/T’ | 0.0007 |
| rs4953262 | PRKCE | 5581 | 2 | 45952444 | ‘A/G’ | 0.0005 |
| rs3886870 | PRKCE | 5581 | 2 | 45954129 | ‘A/G’ | 0.0001 |
| rs935672 | PRKCE | 5581 | 2 | 45957610 | ‘C/T’ | 0.0003 |
| rs4953266 | PRKCE | 5581 | 2 | 45965664 | ‘A/G’ | 0.0001 |
| rs10175198 | PRKCE | 5581 | 2 | 45965765 | ‘A/G’ | 0.0001 |
| rs10175158 | PRKCE | 5581 | 2 | 45965912 | ‘C/T’ | 0.0005 |
| rs2395845 | | | 2 | 222818820 | ‘A/C’ | 0.0006 |
| rs1370923 | | | 2 | 222825345 | ‘A/G’ | 0.0001 |
| rs13385121 | | | 2 | 222828210 | ‘A/G’ | 0.0003 |
| rs1370920 | | | 2 | 222830630 | ‘A/C’ | 0.0007 |
| rs358830 | | | 4 | 21264567 | ‘A/G’ | 0.0006 |
| rs1394135 | | | 4 | 21274584 | ‘C/T’ | 0.0002 |
| rs1394139 | | | 4 | 21283327 | ‘C/T’ | 0.0001 |
| rs3913663 | | | 4 | 21283866 | ‘G/T’ | 0.0001 |
| rs1503994 | | | 4 | 21289743 | ‘C/T’ | 0.0001 |
| rs10000010 | | | 4 | 21294943 | ‘C/T’ | 0.0001 |
| rs6812187 | | | 4 | 21295968 | ‘C/T’ | 0.0002 |
| rs1105377 | | | 4 | 21300252 | ‘A/G’ | 0.0004 |
| rs12523677 | | | 6 | 138972758 | ‘C/T’ | 0.0009 |
| rs7761956 | | | 6 | 138976745 | ‘A/C’ | 0.001 |
| rs9495159 | | | 6 | 138981368 | ‘A/C’ | 0.0003 |
| rs6931390 | | | 6 | 138983096 | ‘A/G’ | 0.0001 |
| rs10085294 | | | 6 | 138983780 | ‘A/G’ | 0.0004 |
| rs7841080 | | | 8 | 33547819 | ‘A/G’ | 0.0007 |
| rs1530344 | | | 8 | 33562618 | ‘C/T’ | 0.0003 |
| rs7814270 | | | 8 | 33590441 | ‘A/G’ | 0.0001 |
| rs10107668 | | | 8 | 33643721 | ‘C/T’ | 0.0006 |
| rs1579274 | | | 8 | 41778080 | ‘G/T’ | 0.0008 |
| rs10103618 | | | 8 | 41783053 | ‘A/G’ | 0.0004 |
| rs1549064 | | | 8 | 41803645 | ‘A/C’ | 0.0001 |
| rs2102360 | | | 8 | 41807985 | ‘A/G’ | 0.0008 |
| rs10501022 | | | 11 | 25712397 | ‘C/T’ | 0.0003 |
| rs2349308 | | | 11 | 25729898 | ‘A/G’ | 0.0004 |
| rs12274588 | | | 11 | 25733789 | ‘A/G’ | 0.0002 |
| rs1493663 | | | 11 | 25735615 | ‘C/T’ | 0.0002 |
| rs1908162 | | | 11 | 25753546 | ‘A/C’ | 0.0001 |
| rs1018022 | | | 11 | 25763400 | ‘A/G’ | 0.0001 |
| rs813321 | LOC441629 | 441629 | 12 | 10774689 | ‘A/G’ | 0.001 |
| rs753202 | LOC441629 | 441629 | 12 | 10777130 | ‘C/T’ | 0.0001 |
| rs797175 | LOC441629 | 441629 | 12 | 10785837 | ‘A/G’ | 0.0002 |
| rs155010 | PCDH9 | 5101 | 13 | 66489493 | ‘C/T’ | 0.0008 |
| rs260172 | PCDH9 | 5101 | 13 | 66496347 | ‘G/T’ | 0.0008 |
| rs7995254 | PCDH9 | 5101 | 13 | 66501746 | ‘C/T’ | 0.0001 |
| rs260148 | PCDH9 | 5101 | 13 | 66505392 | ‘G/T’ | 0.0002 |
| rs1927812 | PCDH9 | 5101 | 13 | 66596990 | ‘C/T’ | 0.0008 |
| rs17517037 | PCDH9 | 5101 | 13 | 66597199 | ‘C/T’ | 0.0001 |
| rs2209902 | PCDH9 | 5101 | 13 | 66600753 | ‘C/T’ | 0.0001 |
| rs1543618 | PCDH9 | 5101 | 13 | 66606571 | ‘A/G’ | 0.001 |
| rs7149784 | | | 14 | 96127751 | ‘A/G’ | 0.001 |
| rs4905507 | | | 14 | 96135250 | ‘A/C’ | 0.0004 |
| rs1570558 | | | 14 | 96141360 | ‘C/T’ | 0.0002 |
| rs234605 | | | 14 | 96141802 | ‘A/G’ | 0.0001 |
| rs6587312 | TAFA5 | 25817 | 22 | 47458658 | ‘A/G’ | 0.0005 |
| rs132262 | TAFA5 | 25817 | 22 | 47462572 | ‘A/G’ | 0.0001 |
| rs131969 | | | 22 | 47472166 | ‘A/G’ | 0.0001 |
| rs13057753 | | | 22 | 47476180 | ‘C/T’ | 0.0001 |
| rs131003 | | | 22 | 47485954 | ‘G/T’ | 0.0001 |
| rs17177527 | | | 22 | 47487073 | ‘A/G’ | 0.0003 |
| rs10521553 | LHFPL1 | 340596 | X | 111687038 | ‘C/T’ | 0.0009 |
| rs7050419 | LHFPL1 | 340596 | X | 111690411 | ‘C/T’ | 0.0003 |
| rs12687789 | LHFPL1 | 340596 | X | 111702031 | ‘G/T’ | 0.0002 |
| rs2851733 | GRIA3 | 2892 | X | 122316606 | ‘A/G’ | 0.0007 |
| rs592807 | GRIA3 | 2892 | X | 122317191 | ‘C/T’ | 0.0002 |
| rs503118 | GRIA3 | 2892 | X | 122319758 | ‘C/T’ | 0.0003 |
| rs5910006 | GRIA3 | 2892 | X | 122341190 | ‘C/T’ | 0.0002 |
| rs4546784 | LOC392533 | 392533 | X | 122355199 | ‘A/G’ | 0.0002 |
| rs5911634 | LOC392533 | 392533 | X | 122359484 | ‘A/C’ | 0.0003 |
| rs1815919 | LOC392533 | 392533 | X | 122361044 | ‘A/G’ | 0.0006 |
| rs5911644 | LOC392533 | 392533 | X | 122370314 | ‘A/G’ | 0.0006 |
| rs930631 | | | X | 145902706 | ‘C/T’ | 0.0006 |
| rs5951926 | | | X | 145903446 | ‘A/G’ | 0.0002 |
| rs12851378 | | | X | 145938989 | ‘C/T’ | 0.0002 |
| rs12156967 | | | X | 145942822 | ‘C/T’ | 0.0002 |
| rs5951934 | | | X | 145950994 | ‘A/G’ | 0.0008 |
| rs5904725 | | | X | 146024839 | ‘C/T’ | 0.001 |
| rs2780882 | | | 1 | 63117448 | ‘A/C’ | 0.0004 |
| rs2780883 | | | 1 | 63122640 | ‘A/G’ | 0.0005 |
| rs2065585 | | | 1 | 63126422 | ‘A/G’ | 0.0002 |
| rs2050249 | | | 1 | 63127755 | ‘A/C’ | 0.0003 |
| rs7559122 | LOC391353 | 391353 | 2 | 16287758 | ‘A/G’ | 0.0002 |
| rs7560874 | LOC391353 | 391353 | 2 | 16291356 | ‘A/G’ | 0.0002 |
| rs2048874 | FLJ40629 | 150468 | 2 | 113240198 | ‘C/T’ | 0.0002 |
| rs4848300 | | | 2 | 113244137 | ‘C/T’ | 0.0002 |
| rs17561 | IL1A | 3552 | 2 | 113253454 | ‘G/T’ | 0.0002 |
| rs6746923 | | | 2 | 113269657 | ‘A/G’ | 0.0003 |
| rs10496444 | | | 2 | 113269899 | ‘C/T’ | 0.0004 |
| rs4849122 | | | 2 | 113277152 | ‘A/G’ | 0.0002 |
| rs4849123 | | | 2 | 113285270 | ‘C/T’ | 0.0002 |
| rs12469600 | | | 2 | 113288588 | ‘C/T’ | 0.0004 |
| rs7630843 | | | 3 | 198681 | ‘C/T’ | 0.0005 |
| rs1850264 | | | 3 | 201067 | ‘A/G’ | 0.0005 |
| rs7632811 | | | 3 | 209726 | ‘G/T’ | 0.001 |
| rs1516338 | CHL1 | 10752 | 3 | 211759 | ‘C/T’ | 0.0007 |
| rs17329247 | CHL1 | 10752 | 3 | 216913 | ‘A/G’ | 0.0005 |
| rs6769747 | CHL1 | 10752 | 3 | 219949 | ‘A/G’ | 0.0002 |
| rs9809528 | CHL1 | 10752 | 3 | 225758 | ‘A/G’ | 0.0005 |
| rs4685447 | CHL1 | 10752 | 3 | 227068 | ‘A/C’ | 0.0002 |
| rs7831515 | FLJ32440 | 286053 | 8 | 126201998 | ‘C/T’ | 0.0007 |
| rs10094316 | FLJ32440 | 286053 | 8 | 126219985 | ‘C/T’ | 0.0004 |
| rs13253942 | FLJ32440 | 286053 | 8 | 126223831 | ‘A/G’ | 0.0002 |
| rs12544146 | FLJ32440 | 286053 | 8 | 126241522 | ‘A/G’ | 0.0004 |
| rs10093813 | FLJ32440 | 286053 | 8 | 126286445 | ‘C/T’ | 0.0002 |
| rs4330708 | FLJ32440 | 286053 | 8 | 126302381 | ‘G/T’ | 0.0004 |
| rs3955404 | FLJ32440 | 286053 | 8 | 126323441 | ‘C/T’ | 0.0007 |
| rs4749567 | | | 10 | 30885044 | ‘A/G’ | 0.0008 |
| rs4749568 | | | 10 | 30887013 | ‘C/T’ | 0.0004 |
| rs906236 | | | 10 | 30891225 | ‘A/C’ | 0.0002 |
| rs12099631 | | | 12 | 83139088 | ‘A/G’ | 0.0009 |
| rs728084 | | | 12 | 83170810 | ‘A/G’ | 0.0008 |
| rs2279307 | | | 12 | 83190484 | ‘C/T’ | 0.0002 |
| rs1564606 | | | 12 | 83199601 | ‘G/T’ | 0.0005 |
| rs9538278 | | | 13 | 58510378 | ‘A/G’ | 0.0003 |
| rs7333943 | | | 13 | 58515848 | ‘G/T’ | 0.0002 |
| rs6562004 | | | 13 | 58517819 | ‘A/G’ | 0.0006 |
| rs803804 | | | 13 | 70497303 | ‘A/G’ | 0.0006 |
| rs9542557 | | | 13 | 70507666 | ‘A/G’ | 0.0002 |
| rs1395354 | | | 13 | 70514920 | ‘A/G’ | 0.0002 |
| rs2135488 | | | 13 | 70515776 | ‘C/T’ | 0.0006 |
| rs1683378 | FLJ34907 | 284222 | 18 | 10833483 | ‘C/T’ | 0.0009 |
| rs901185 | FLJ34907 | 284222 | 18 | 10844509 | ‘C/T’ | 0.0002 |
| rs11874473 | FLJ34907 | 284222 | 18 | 10853849 | ‘A/C’ | 0.0004 |
| rs11659801 | FLJ34907 | 284222 | 18 | 10858838 | ‘A/G’ | 0.0004 |
| rs196956 | | | 18 | 10882653 | ‘A/G’ | 0.0005 |
| rs264167 | | | 18 | 10886327 | ‘G/T’ | 0.0009 |
| rs264176 | | | 18 | 10891607 | ‘C/T’ | 0.0009 |
| rs12012576 | | | X | 21572835 | ‘A/G’ | 0.0003 |
| rs7878576 | | | X | 21596302 | ‘A/G’ | 0.0004 |
| rs5951469 | | | X | 21608265 | ‘C/T’ | 0.0009 |
| rs2224075 | DMD | 1756 | X | 32596618 | ‘G/T’ | 0.0004 |
| rs1015377 | DMD | 1756 | X | 32610610 | ‘A/C’ | 0.0003 |
| rs5972689 | DMD | 1756 | X | 32619678 | ‘A/G’ | 0.0005 |
| rs5937044 | | | X | 69983714 | ‘A/G’ | 0.001 |
| rs5937060 | | | X | 70024726 | ‘C/T’ | 0.0003 |
| rs3125945 | | | X | 70041757 | ‘A/G’ | 0.0007 |
| rs12841491 | BRODL | 254065 | X | 79835631 | ‘A/G’ | 0.0003 |
| rs1997686 | | | X | 141888741 | ‘C/T’ | 0.0007 |
| rs5908533 | | | X | 141889739 | ‘C/T’ | 0.0004 |
| rs5907387 | | | X | 141893710 | ‘C/T’ | 0.0003 |
| rs5951913 | | | X | 145841872 | ‘A/G’ | 0.0007 |
| rs4460510 | | | X | 145845745 | ‘G/T’ | 0.0003 |
| rs6535510 | | | 4 | 85188903 | ‘C/T’ | 0.0003 |
| rs1395000 | | | 4 | 85197861 | ‘A/G’ | 0.0004 |
| rs1827814 | | | 4 | 85212132 | ‘A/G’ | 0.0006 |
| rs4423888 | | | 4 | 125833487 | ‘A/C’ | 0.0003 |
| rs2318064 | TCBA1 | 154215 | 6 | 124231122 | ‘A/G’ | 0.0008 |
| rs6924068 | TCBA1 | 154215 | 6 | 124232587 | ‘A/G’ | 0.0003 |
| rs11154196 | TCBA1 | 154215 | 6 | 124259412 | ‘A/G’ | 0.0007 |
| rs1373762 | | | 18 | 48008075 | ‘A/G’ | 0.0004 |
| rs1445097 | | | 18 | 48008752 | ‘A/G’ | 0.0003 |
| rs920938 | | | 18 | 48031307 | ‘A/C’ | 0.0004 |
| rs7238445 | | | 18 | 48035542 | ‘A/G’ | 0.0008 |
| rs2839081 | | | 21 | 46265743 | ‘C/T’ | 0.0003 |
| rs2839084 | | | 21 | 46269612 | ‘C/T’ | 0.0009 |
| rs12856241 | | | X | 42617791 | ‘C/T’ | 0.0007 |
| rs11797347 | | | X | 42619916 | ‘A/G’ | 0.0004 |
| rs2497938 | LOC442457 | 442457 | X | 66346039 | ‘C/T’ | 0.0004 |
| rs6625187 | LOC442457 | 442457 | X | 66459416 | ‘C/T’ | 0.0009 |
| rs1716758 | | | X | 117241790 | ‘A/G’ | 0.0007 |
| rs5910338 | | | X | 117242500 | ‘C/T’ | 0.0004 |
| rs5910340 | | | X | 117247923 | ‘C/T’ | 0.0007 |
| rs1781994 | | | X | 117251385 | ‘A/G’ | 0.001 |
| rs13013240 | | | 2 | 154378937 | ‘A/G’ | 0.0004 |
| rs2594264 | FHIT | 2272 | 3 | 60489776 | ‘A/G’ | 0.0007 |
| rs717821 | FHIT | 2272 | 3 | 60490818 | ‘C/T’ | 0.0004 |
| rs4688500 | | | 3 | 64651333 | ‘C/T’ | 0.001 |
| rs10470707 | | | 3 | 64652777 | ‘A/G’ | 0.0004 |
| rs4234678 | | | 3 | 64654405 | ‘C/T’ | 0.0006 |
| rs7648557 | | | 3 | 64658255 | ‘G/T’ | 0.0004 |
| rs9828674 | | | 3 | 64668140 | ‘G/T’ | 0.0004 |
| rs6534743 | | | 4 | 131072373 | ‘A/G’ | 0.0004 |
| rs1470968 | | | 8 | 115879414 | ‘A/C’ | 0.0007 |
| rs1013527 | | | 8 | 115890222 | ‘G/T’ | 0.0005 |
| rs13252246 | | | 8 | 115890469 | ‘A/G’ | 0.0004 |
| rs10505228 | | | 8 | 115908486 | ‘C/T’ | 0.0006 |
| rs7901450 | | | 10 | 120200634 | ‘G/T’ | 0.0004 |
| rs2040322 | NELL1 | 4745 | 11 | 21319903 | ‘A/G’ | 0.0004 |
| rs10833511 | NELL1 | 4745 | 11 | 21321150 | ‘G/T’ | 0.0006 |
| rs6483768 | NELL1 | 4745 | 11 | 21328115 | ‘A/C’ | 0.0008 |
| rs12558663 | | | X | 98465719 | ‘G/T’ | 0.0005 |
| rs5955985 | | | X | 116802803 | ‘A/G’ | 0.0007 |
| rs5910260 | | | X | 116803867 | ‘A/C’ | 0.0005 |
| rs6603347 | KLHL13 | 90293 | X | 116839006 | ‘A/G’ | 0.0005 |
| rs7880254 | | | X | 129289361 | ‘A/G’ | 0.0008 |
| rs2411857 | | | X | 129305419 | ‘C/T’ | 0.0007 |
| rs5977297 | | | X | 129333622 | ‘C/T’ | 0.0006 |
| rs5977301 | | | X | 129344002 | ‘A/C’ | 0.0005 |
| rs4830190 | | | X | 129348212 | ‘A/G’ | 0.0006 |
| rs4926448 | CGI-49 | 51097 | 1 | 243252908 | ‘C/T’ | 0.0006 |
| rs4926440 | CGI-49 | 51097 | 1 | 243255059 | ‘C/T’ | 0.0007 |
| rs6694274 | LOC149134 | 149134 | 1 | 243280207 | ‘A/G’ | 0.0005 |
| rs10027062 | | | 4 | 172681785 | ‘A/G’ | 0.0005 |
| rs12649451 | | | 4 | 172690498 | ‘C/T’ | 0.0009 |
| rs12184555 | PCDH17 | 27253 | 13 | 57180524 | ‘C/T’ | 0.0005 |
| rs10498645 | | | 14 | 95332505 | ‘A/G’ | 0.0009 |
| rs6575549 | | | 14 | 95333063 | ‘C/T’ | 0.0005 |
| rs1957923 | | | 14 | 95344355 | ‘A/G’ | 0.0007 |
| rs4786026 | NPM1P3 | 4872 | 16 | 5355972 | ‘A/G’ | 0.0007 |
| rs9929602 | | | 16 | 5363159 | ‘A/C’ | 0.0006 |
| rs485335 | | | 16 | 5365169 | ‘A/G’ | 0.0005 |
| rs507215 | | | 16 | 5368557 | ‘C/T’ | 0.0005 |
| rs2870478 | | | 19 | 62080707 | ‘A/C’ | 0.0005 |
| rs5917070 | LOC392517 | 392517 | X | 106840233 | ‘A/G’ | 0.0009 |
| rs2300101 | MID2 | 11043 | X | 106947702 | ‘C/T’ | 0.001 |
| rs5916793 | MID2 | 11043 | X | 106953693 | ‘A/C’ | 0.0006 |
| rs5931610 | | | X | 138141139 | ‘A/C’ | 0.0007 |
| rs6418811 | | | X | 138161892 | ‘A/G’ | 0.0006 |
| rs9388813 | | | 6 | 130965020 | ‘A/G’ | 0.0006 |
| rs564127 | | | 7 | 79540870 | ‘C/T’ | 0.0006 |
| rs2396104 | | | 7 | 108701750 | ‘C/T’ | 0.0006 |
| rs7016063 | | | 8 | 55568807 | ‘C/T’ | 0.0006 |
| rs7909332 | | | 10 | 109509005 | ‘A/G’ | 0.0006 |
| rs1023033 | PCDH9 | 5101 | 13 | 66544115 | ‘A/G’ | 0.0009 |
| rs1927822 | PCDH9 | 5101 | 13 | 66547600 | ‘C/T’ | 0.0006 |
| rs10873145 | | | 14 | 61749543 | ‘A/G’ | 0.0006 |
| rs1104708 | | | 14 | 61758158 | ‘A/G’ | 0.0007 |
| rs9951631 | DSC1 | 1823 | 18 | 26995905 | ‘C/T’ | 0.0006 |
| rs6018359 | PRKCBP1 | 23613 | 20 | 45307330 | ‘C/T’ | 0.0006 |
| rs761021 | PRKCBP1 | 23613 | 20 | 45327366 | ‘C/T’ | 0.0006 |
| rs8132319 | NCAM2 | 4685 | 21 | 21349566 | ‘C/T’ | 0.0006 |
| rs7058356 | | | X | 5252012 | ‘A/G’ | 0.0007 |
| rs4826788 | | | X | 5305855 | ‘C/T’ | 0.0008 |
| rs12009051 | | | X | 45883560 | ‘C/T’ | 0.0007 |
| rs2043072 | CNTNAP5 | 129684 | 2 | 124873071 | ‘C/T’ | 0.001 |
| rs2584353 | CNTNAP5 | 129684 | 2 | 124875522 | ‘G/T’ | 0.0007 |
| rs2964911 | | | 5 | 163656859 | ‘C/T’ | 0.0007 |
| rs10250289 | KIAA1862 | 84626 | 7 | 148783329 | ‘A/G’ | 0.0009 |
| rs731489 | KIAA1862 | 84626 | 7 | 148798930 | ‘A/G’ | 0.0007 |
| rs11780975 | | | 8 | 103536662 | ‘A/C’ | 0.0007 |
| rs777801 | | | 8 | 116245300 | ‘C/T’ | 0.0007 |
| rs1888952 | | | 9 | 16248118 | ‘C/T’ | 0.0007 |
| rs10756747 | | | 9 | 16249346 | ‘A/G’ | 0.001 |
| rs10120750 | | | 9 | 87215060 | ‘A/G’ | 0.0007 |
| rs574322 | HNT | 50863 | 11 | 131326210 | ‘C/T’ | 0.0007 |
| rs1022866 | PCDH9 | 5101 | 13 | 66480115 | ‘A/G’ | 0.0007 |
| rs11646540 | PRKCB1 | 5579 | 16 | 24031685 | ‘A/G’ | 0.0007 |
| rs1995171 | | | 16 | 50137027 | ‘A/C’ | 0.0007 |
| rs4784368 | | | 16 | 50151691 | ‘C/T’ | 0.0008 |
| rs1189852 | FLJ34907 | 284222 | 18 | 10817757 | ‘C/T’ | 0.0007 |
| rs7504149 | | | 18 | 63985504 | ‘A/C’ | 0.0009 |
| rs491920 | | | 18 | 63993805 | ‘G/T’ | 0.0007 |
| rs758119 | DXS1283E | 8228 | X | 7697726 | ‘A/G’ | 0.0008 |
| rs1795600 | | | X | 7716680 | ‘C/T’ | 0.0008 |
| rs4829455 | AMOT | 154796 | X | 111870035 | ‘A/C’ | 0.0008 |
| rs5973962 | AMOT | 154796 | X | 111873526 | ‘C/T’ | 0.001 |
| rs620730 | AMOT | 154796 | X | 111879310 | ‘C/T’ | 0.0008 |
| rs10913257 | PAPPA2 | 60676 | 1 | 173510267 | ‘G/T’ | 0.0008 |
| rs7607623 | | | 2 | 35641207 | ‘A/G’ | 0.0008 |
| rs849523 | NRP2 | 8828 | 2 | 206421442 | ‘C/T’ | 0.0008 |
| rs10498133 | PAX3 | 5077 | 2 | 222929810 | ‘G/T’ | 0.0008 |
| rs2134358 | CNTN4 | 152330 | 3 | 2447785 | ‘C/T’ | 0.0008 |
| rs9838361 | HAPIP | 8997 | 3 | 125517439 | ‘G/T’ | 0.0008 |
| rs9819507 | | | 3 | 185249403 | ‘C/T’ | 0.0008 |
| rs2642749 | LOC389293 | 389293 | 5 | 62137231 | ‘A/G’ | 0.0008 |
| rs12523684 | KIAA1900 | 114792 | 6 | 97536824 | ‘A/G’ | 0.0008 |
| rs1933459 | KIAA1900 | 114792 | 6 | 97545010 | ‘A/G’ | 0.0009 |
| rs1019906 | DGKB | 1607 | 7 | 14176024 | ‘C/T’ | 0.0008 |
| rs2194910 | | | 8 | 54250695 | ‘C/T’ | 0.0009 |
| rs7007275 | | | 8 | 54269163 | ‘G/T’ | 0.0008 |
| rs10511739 | | | 9 | 24081342 | ‘A/G’ | 0.001 |
| rs4977917 | | | 9 | 24095508 | ‘A/G’ | 0.0008 |
| rs937872 | | | 13 | 68252838 | ‘A/G’ | 0.0008 |
| rs759290 | PPP5C | 5536 | 19 | 51583951 | ‘C/T’ | 0.0008 |
| rs2288419 | PTPRH | 5794 | 19 | 60385056 | ‘A/G’ | 0.0009 |
| rs2288523 | PTPRH | 5794 | 19 | 60394722 | ‘G/T’ | 0.0008 |
| rs504507 | | | 20 | 874585 | ‘A/G’ | 0.0008 |
| rs530652 | | | 20 | 878560 | ‘C/T’ | 0.0008 |
| rs2824669 | | | 21 | 18457462 | ‘A/C’ | 0.001 |
| rs909260 | | | 21 | 18459878 | ‘A/G’ | 0.0008 |
| rs2186343 | | | 21 | 38258803 | ‘G/T’ | 0.0008 |
| rs1539902 | | | 21 | 38259079 | ‘A/G’ | 0.0008 |
| rs2983097 | | | X | 102472426 | ‘A/G’ | 0.0009 |
| rs7539699 | | | 1 | 244075613 | ‘A/G’ | 0.0009 |
| rs997448 | | | 3 | 64935253 | ‘A/G’ | 0.0009 |
| rs562 | ABCC5 | 10057 | 3 | 185120547 | ‘C/T’ | 0.0009 |
| rs3109915 | | | 4 | 55480475 | ‘A/G’ | 0.0009 |
| rs628572 | | | 6 | 16873481 | ‘A/G’ | 0.0009 |
| rs10097861 | PTK2B | 2185 | 8 | 27244435 | ‘A/G’ | 0.0009 |
| rs1879188 | PTK2B | 2185 | 8 | 27249840 | ‘G/T’ | 0.0009 |
| rs723231 | | | 8 | 126018234 | ‘G/T’ | 0.0009 |
| rs7025486 | | | 9 | 121501957 | ‘A/G’ | 0.0009 |
| rs7320321 | | | 13 | 105089064 | ‘A/G’ | 0.0009 |
| rs158074 | | | 21 | 18303801 | ‘C/T’ | 0.0009 |
| rs157740 | | | 21 | 18325093 | ‘A/G’ | 0.0009 |
| rs4911823 | | | X | 114478198 | ‘C/T’ | 0.001 |
| rs1029307 | | | X | 138041664 | ‘C/T’ | 0.001 |
| rs6683479 | | | 1 | 190196142 | ‘A/G’ | 0.001 |
| rs2551640 | CREB1 | 1385 | 2 | 208233399 | ‘A/G’ | 0.001 |
| rs2244503 | | | 3 | 64920911 | ‘C/T’ | 0.001 |
| rs11926273 | | | 3 | 149211480 | ‘C/T’ | 0.001 |
| rs7613237 | | | 3 | 185223836 | ‘C/T’ | 0.001 |
| rs1461656 | LOC340156 | 340156 | 6 | 2661859 | ‘A/G’ | 0.001 |
| rs1708552 | | | 6 | 67101152 | ‘G/T’ | 0.001 |
| rs911946 | SMOC2 | 64094 | 6 | 168861132 | ‘C/T’ | 0.001 |
| rs4518582 | | | 7 | 135798488 | ‘C/T’ | 0.001 |
| rs7830593 | | | 8 | 23000640 | ‘A/G’ | 0.001 |
| rs10867485 | | | 9 | 80104890 | ‘A/G’ | 0.001 |
| rs3012797 | | | 9 | 135049961 | ‘A/G’ | 0.001 |
| rs2382712 | | | 9 | 135050485 | ‘C/T’ | 0.001 |
| rs7984277 | | | 13 | 57299434 | ‘A/G’ | 0.001 |
| rs2060261 | FLJ39501 | 126410 | 19 | 15482180 | ‘A/G’ | 0.001 |

dbSNP_rs_ID: SNP identification number in NCBI dbSNP database
Sequence_ID: Sequence identification number
Gene_locus: Gene locus and gene id as reported by NCBI dbSNP database build 126
Variants: Alternate SNP alleles or their complementary nucleotides in the position indicated by dbSNP RS ID and basepair position
P-value: P-value based on permutation test
Position: Basepair Position, SNP physical position according to NCBI Human Genome Build 35.1
Gene_content: Genes positioned within 100 Kbp up and downstream (End) from the physical position of the SNPs bordering the haplotype genomic region based on NCBI Human Genome Build 36.1

TABLE 4
Haplotypes with the strongest association with HT based on HaploRec + HPM analysis with 8
SNPs. The analysis is based on 140 HT cases and 182 healthy controls from East Finland.

| | Gene locus | | | | | | | |
|---|---|---|---|---|---|---|---|---|
| | and | | | | | | | |
| dbSNP rs ID | Gene ID | | Chromosome | Position | Variats | Risk Allele | Chi square | P value |
| rs7539699 | | | 1 | 244075613 | ‘A/G’ | G | 24.69 | 6.74E−07 |
| rs10925085 | OR2G3 | 81469 | 1 | 244078202 | ‘C/T’ | A | | |
| rs869111 | OR2G3 | 81469 | 1 | 244078408 | ‘A/G’ | G | | |
| rs10489818 | | | 1 | 117811285 | ‘A/G’ | G | 20.71 | 5.35E−06 |
| rs6661142 | | | 1 | 117818118 | ‘C/T’ | A | | |
| rs4659053 | | | 1 | 117820901 | ‘G/T’ | C | | |
| rs1963278 | | | 1 | 117827285 | ‘A/G’ | A | | |
| rs4261104 | | | 1 | 117831576 | ‘C/T’ | A | | |
| rs1877341 | ALS2CR19 | 117583 | 2 | 206173074 | ‘C/T’ | A | 20.26 | 6.77E−06 |
| rs759450 | ALS2CR19 | 117583 | 2 | 206185110 | ‘A/G’ | G | | |
| rs12474620 | ALS2CR19 | 117583 | 2 | 206199530 | ‘A/G’ | G | | |
| rs992159 | ALS2CR19 | 117583 | 2 | 206217051 | ‘A/C’ | C | | |
| rs2041832 | ALS2CR19 | 117583 | 2 | 206262643 | ‘A/G’ | G | | |
| rs6731822 | FLJ40629 | 150468 | 2 | 113230056 | ‘C/T’ | G | 19.78 | 8.68E−06 |
| rs2048874 | FLJ40629 | 150468 | 2 | 113240198 | ‘C/T’ | G | | |
| rs4848300 | | | 2 | 113244137 | ‘C/T’ | A | | |
| rs17561 | IL1A | 3552 | 2 | 113253454 | ‘G/T’ | C | | |
| rs7648557 | | | 3 | 64658255 | ‘G/T’ | A | 23.65 | 1.16E−06 |
| rs9828674 | | | 3 | 64668140 | ‘G/T’ | C | | |
| rs11936235 | LOC152742 | 152742 | 4 | 13794650 | ‘C/T’ | A | 21.33 | 3.86E−06 |
| rs3846401 | LOC152742 | 152742 | 4 | 13803752 | ‘A/G’ | A | | |
| rs3846407 | LOC152742 | 152742 | 4 | 13808194 | ‘G/T’ | A | | |
| rs7654692 | LOC152742 | 152742 | 4 | 13810072 | ‘A/G’ | G | | |
| rs1426123 | LOC152742 | 152742 | 4 | 13814053 | ‘A/C’ | C | | |
| rs3846413 | LOC152742 | 152742 | 4 | 13816480 | ‘C/T’ | A | | |
| rs4698716 | | | 4 | 13824643 | ‘C/T’ | A | | |
| rs3846415 | | | 4 | 13829206 | ‘A/G’ | A | | |
| rs9640521 | CNTNAP2 | 26047 | 7 | 147429304 | ‘G/T’ | A | 20.75 | 5.24E−06 |
| rs13244714 | CNTNAP2 | 26047 | 7 | 147433108 | ‘A/G’ | A | | |
| rs12673933 | CNTNAP2 | 26047 | 7 | 147441753 | ‘C/T’ | A | | |
| rs6981891 | LOC392222 | 392222 | 8 | 55554449 | ‘A/G’ | A | 22.96 | 1.65E−06 |
| rs16920368 | LOC392222 | 392222 | 8 | 55555406 | ‘A/G’ | A | | |
| rs10504166 | LOC392222 | 392222 | 8 | 55556364 | ‘G/T’ | C | | |
| rs11786806 | | | 8 | 55561942 | ‘C/T’ | A | | |
| rs7830517 | | | 8 | 55566337 | ‘A/G’ | G | | |
| rs10109281 | | | 8 | 55568620 | ‘A/G’ | G | | |
| rs7016063 | | | 8 | 55568807 | ‘C/T’ | A | | |
| rs6473938 | | | 8 | 55586240 | ‘C/T’ | A | | |
| rs7814270 | | | 8 | 33590441 | ‘A/G’ | G | 22.15 | 2.52E−06 |
| rs10107668 | | | 8 | 33643721 | ‘C/T’ | A | | |
| rs6981979 | ANK1 | 286 | 8 | 41731412 | ‘C/T’ | A | 20.49 | 5.98E−06 |
| rs11997827 | ANK1 | 286 | 8 | 41741060 | ‘C/T’ | G | | |
| rs11780780 | ANK1 | 286 | 8 | 41742759 | ‘A/G’ | G | | |
| rs13255458 | ANK1 | 286 | 8 | 41755228 | ‘C/T’ | G | | |
| rs879638 | ANK1 | 286 | 8 | 41770625 | ‘C/T’ | A | | |
| rs1579274 | | | 8 | 41778080 | ‘G/T’ | C | | |
| rs10103618 | | | 8 | 41783053 | ‘A/G’ | A | | |
| rs1549064 | | | 8 | 41803645 | ‘A/C’ | C | | |
| rs16922271 | NCOA6IP | 96764 | 8 | 56885245 | ‘C/T’ | G | 20.14 | 7.21E−06 |
| rs12155521 | | | 8 | 56920362 | ‘G/T’ | A | | |
| rs12676220 | | | 8 | 56923173 | ‘C/T’ | A | | |
| rs574847 | | | 12 | 91259931 | ‘C/T’ | G | 20.55 | 5.81E−06 |
| rs427560 | | | 12 | 91279485 | ‘A/C’ | A | | |
| rs3890018 | | | 12 | 91282738 | ‘G/T’ | A | | |
| rs337653 | | | 12 | 91296145 | ‘C/T’ | A | | |
| rs389714 | | | 12 | 91299920 | ‘A/G’ | G | | |
| rs7328290 | | | 13 | 71394581 | ‘A/G’ | A | 20.93 | 4.75E−06 |
| rs9542777 | | | 13 | 71394590 | ‘A/G’ | A | | |
| rs1571393 | PCDH9 | 5101 | 13 | 66560186 | ‘A/G’ | A | 20.09 | 7.39E−06 |
| rs9529185 | PCDH9 | 5101 | 13 | 66572148 | ‘A/G’ | G | | |
| rs9317636 | PCDH9 | 5101 | 13 | 66573353 | ‘C/T’ | A | | |
| rs17516342 | PCDH9 | 5101 | 13 | 66580629 | ‘C/T’ | A | | |
| rs1927825 | PCDH9 | 5101 | 13 | 66586299 | ‘A/G’ | G | | |
| rs1927826 | PCDH9 | 5101 | 13 | 66588057 | ‘A/G’ | G | | |
| rs9571713 | PCDH9 | 5101 | 13 | 66591183 | ‘C/T’ | A | | |
| rs2875517 | PCDH9 | 5101 | 13 | 66594146 | ‘A/G’ | A | | |
| rs260172 | PCDH9 | 5101 | 13 | 66496347 | ‘G/T’ | A | 19.78 | 8.71E−06 |
| rs7995254 | PCDH9 | 5101 | 13 | 66501746 | ‘C/T’ | A | | |
| rs2147829 | TTC7B | 145567 | 14 | 90233622 | ‘C/T’ | A | 20.28 | 6.70E−06 |
| rs3814841 | TTC7B | 145567 | 14 | 90234219 | ‘C/T’ | G | | |
| rs1742098 | TTC7B | 145567 | 14 | 90238170 | ‘C/T’ | A | | |
| rs1749704 | TTC7B | 145567 | 14 | 90239107 | ‘G/T’ | A | | |
| rs1535321 | TTC7B | 145567 | 14 | 90240579 | ‘C/T’ | A | | |
| rs1749718 | TTC7B | 145567 | 14 | 90253080 | ‘C/T’ | G | | |
| rs1742083 | TTC7B | 145567 | 14 | 90256423 | ‘C/T’ | A | | |
| rs8018904 | TTC7B | 145567 | 14 | 90259730 | ‘G/T’ | C | | |
| rs901185 | FLJ34907 | 284222 | 18 | 10844509 | ‘C/T’ | G | 27.87 | 1.30E−07 |
| rs11874473 | FLJ34907 | 284222 | 18 | 10853849 | ‘A/C’ | A | | |
| rs11659801 | FLJ34907 | 284222 | 18 | 10858838 | ‘A/G’ | A | | |
| rs196956 | | | 18 | 10882653 | ‘A/G’ | G | | |
| rs1189852 | FLJ34907 | 284222 | 18 | 10817757 | ‘C/T’ | A | 20.71 | 5.33E−06 |
| rs9962727 | FLJ34907 | 284222 | 18 | 10822851 | ‘C/T’ | A | | |
| rs9807627 | FLJ34907 | 284222 | 18 | 10831164 | ‘A/G’ | G | | |
| rs1683376 | FLJ34907 | 284222 | 18 | 10832934 | ‘A/G’ | G | | |
| rs1683378 | FLJ34907 | 284222 | 18 | 10833483 | ‘C/T’ | G | | |
| rs901185 | FLJ34907 | 284222 | 18 | 10844509 | ‘C/T’ | G | | |
| rs8099113 | NFATC1 | 4772 | 18 | 75367142 | ‘A/G’ | G | 20.33 | 6.53E−06 |
| rs1078633 | NFATC1 | 4772 | 18 | 75369498 | ‘G/T’ | C | | |
| rs372741 | NFATC1 | 4772 | 18 | 75370277 | ‘C/T’ | A | | |
| rs177820 | NFATC1 | 4772 | 18 | 75377952 | ‘C/T’ | A | | |
| rs2044750 | NFATC1 | 4772 | 18 | 75380738 | ‘A/G’ | G | | |
| rs9518 | NFATC1 | 4772 | 18 | 75389794 | ‘C/T’ | A | | |
| rs1437606 | | | 18 | 27019528 | ‘C/T’ | G | 20.17 | 7.10E−06 |
| rs1469945 | | | 18 | 27023130 | ‘C/T’ | A | | |
| rs2919996 | | | 18 | 27023635 | ‘C/T’ | G | | |
| rs4447498 | | | 18 | 27037686 | ‘C/T’ | G | | |
| rs502716 | | | 20 | 874397 | ‘A/G’ | G | 19.78 | 8.67E−06 |
| rs504507 | | | 20 | 874585 | ‘A/G’ | A | | |
| rs530652 | | | 20 | 878560 | ‘C/T’ | A | | |
| rs480789 | | | 20 | 880618 | ‘C/T’ | A | | |
| rs6140734 | | | 20 | 882313 | ‘A/G’ | G | | |
| rs550408 | | | 20 | 882948 | ‘C/T’ | A | | |
| rs2001902 | | | 22 | 46890123 | ‘A/C’ | A | 20.45 | 6.13E−06 |
| rs133519 | | | 22 | 46897247 | ‘G/T’ | C | | |
| rs9615272 | | | 22 | 46901575 | ‘A/G’ | G | | |
| rs941418 | | | 22 | 46905131 | ‘A/G’ | G | | |
| rs926233 | | | 22 | 46908479 | ‘A/G’ | G | | |
| rs6008577 | | | 22 | 46910609 | ‘A/G’ | A | | |
| rs133530 | | | 22 | 46911963 | ‘A/G’ | G | | |
| rs6649483 | | | X | 148947202 | ‘C/T’ | A | 29.35 | 6.05E−08 |
| rs9778461 | | | X | 149023178 | ‘A/G’ | A | | |
| rs3897225 | | | X | 149042894 | ‘A/C’ | A | | |
| rs12394687 | | | X | 149058726 | ‘C/T’ | A | | |
| rs12398405 | | | X | 149062629 | ‘A/C’ | A | | |
| rs9698926 | | | X | 149093381 | ‘C/T’ | A | | |
| rs9781523 | | | X | 149099941 | ‘A/G’ | G | | |
| rs9284560 | | | X | 149104836 | ‘C/T’ | A | | |
| rs12014072 | | | X | 109436852 | ‘A/C’ | A | 29.27 | 6.31E−08 |
| rs10521528 | KIAA1318 | 57529 | X | 109495297 | ‘A/G’ | A | | |
| rs6567866 | | | X | 109509009 | ‘C/T’ | A | | |
| rs5942641 | | | X | 109549379 | ‘A/G’ | G | | |
| rs1573036 | | | X | 109626213 | ‘A/G’ | G | | |
| rs5942651 | | | X | 109633767 | ‘A/G’ | A | | |
| rs197023 | CHRDL1 | 91851 | X | 109774532 | ‘C/T’ | A | | |
| rs12689346 | CHRDL1 | 91851 | X | 109810107 | ‘C/T’ | A | | |

dbSNP_rs_ID: SNP identification number in NCBI dbSNP database
Sequence_ID: Sequence identification number
Gene_locus: Gene locus and gene id as reported by NCBI dbSNP database build 126
Position: Basepair Position, SNP physical position according to NCBI Human Genome Build 36.1
Variants: Alternate SNP alleles or their complementary nucleotides in the position indicated by dbSNP RS ID and basepair position
Risk_allele: Allele in at-risk haplotype
Chi_square: Chi-squared test based on allele frequencies
P-value: P-value based on the chi-square test

TABLE 5
SNP markers with the strongest association with hypertension in the individual marker analysis. The analysis is based on the
combined data of 110 HT cases and 110 healthy controls from the Ashkenazi Jew population, 114 HT cases and 114 healthy controls
from the East Finnish population, 41 HT cases and 41 healthy controls from the German population and 28 HT cases and 28 healthy
controls from the English population.

| | | Gene locus | | | | | | | |
|---|---|---|---|---|---|---|---|---|---|
| | | and | | | | | | | |
| dbSNP rs ID | Priority date | Gene ID | | Chromosome | Position | Variats | Minor Allele | Allele X2 | Odds ratio |
| RS1721355 | 3 | WDR69 | 164781 | 2 | 228491220 | ‘A/G’ | G | 18.72 | 0.60 |
| RS561264 | 3 | | | 2 | 238994718 | ‘A/C’ | C | 18.27 | 1.78 |
| RS2153184 | 3 | | | 1 | 162470621 | ‘A/G’ | A | 17.82 | 1.70 |
| RS9564765 | 3 | | | 13 | 70431786 | ‘A/G’ | G | 17.79 | 1.74 |
| RS8066575 | 3 | FLJ45831 | 400576 | 17 | 14631647 | ‘C/T’ | G | 17.76 | 0.57 |
| RS6698312 | 3 | | | 1 | 162473439 | ‘G/T’ | A | 17.60 | 1.76 |
| RS2301301 | 3 | HOXD3 | 3232 | 2 | 176740513 | ‘C/T’ | G | 16.94 | 1.66 |
| RS7406978 | 3 | ABR | 29 | 17 | 983909 | ‘C/T’ | A | 16.79 | 1.62 |
| RS2245192 | 3 | | | 7 | 113789771 | ‘C/T’ | G | 16.69 | 0.51 |
| RS747250 | 3 | LOC651311 | 651311 | 11 | 129776888 | ‘G/T’ | A | 16.29 | 1.61 |
| RS1332855 | 3 | | | 9 | 82827607 | ‘G/T’ | C | 16.25 | 0.49 |
| RS10516684 | 3 | | | 4 | 84529735 | ‘C/T’ | A | 16.16 | 0.45 |
| RS11650418 | 3 | ABR | 29 | 17 | 1025021 | ‘G/T’ | A | 16.03 | 0.62 |
| RS2256182 | 3 | | | 8 | 93637241 | ‘C/T’ | A | 15.99 | 0.58 |
| RS590218 | 3 | ZNF516 | 9658 | 18 | 72198871 | ‘A/G’ | G | 15.83 | 0.52 |
| RS16928804 | 3 | | | 9 | 128033773 | ‘A/C’ | A | 15.82 | 0.49 |
| RS4399939 | 3 | | | 4 | 162037658 | ‘C/T’ | A | 15.54 | 0.54 |
| RS13100475 | 3 | | | 3 | 192637284 | ‘A/G’ | A | 15.35 | 0.56 |
| RS9546945 | 3 | | | 13 | 84406708 | ‘C/T’ | G | 15.20 | 1.60 |
| RS1183060 | 3 | | | 9 | 82849539 | ‘C/T’ | G | 15.18 | 0.49 |
| RS2881507 | 3 | | | 1 | 162492876 | ‘A/G’ | A | 15.10 | 1.68 |
| RS7141 | 3 | EFNB3 | 1949 | 17 | 7555326 | ‘A/G’ | G | 15.08 | 1.62 |
| RS1464706 | 3 | | | 3 | 947112 | ‘C/T’ | G | 15.08 | 0.59 |
| RS1369704 | 3 | DTNB | 1838 | 2 | 25477094 | ‘A/G’ | G | 15.00 | 0.62 |
| RS2458686 | 3 | | | 10 | 2548037 | ‘G/T’ | A | 14.98 | 0.52 |
| RS1543680 | 3 | HIST1H4C | 8364 | 6 | 26211156 | ‘A/G’ | A | 14.95 | 0.54 |
| RS707889 | 3 | HFE | 3077 | 6 | 26203910 | ‘C/T’ | A | 14.95 | 0.54 |
| RS261988 | 3 | | | 5 | 95866641 | ‘A/G’ | G | 14.65 | 1.57 |
| RS11088668 | 3 | | | 21 | 18448920 | ‘C/T’ | A | 14.65 | 1.97 |
| RS4902242 | 3 | | | 14 | 63299842 | ‘C/T’ | G | 14.43 | 0.52 |
| RS934083 | 3 | CACNA2D3 | 55799 | 3 | 54765419 | ‘C/T’ | G | 14.42 | 1.56 |
| RS1476240 | 3 | ZPBP | 11055 | 7 | 50003183 | ‘A/G’ | G | 14.32 | 1.73 |
| RS8131179 | 3 | PDE9A | 5152 | 21 | 42955270 | ‘C/T’ | A | 14.07 | 0.62 |
| RS941223 | 3 | | | 12 | 19912392 | ‘A/G’ | G | 13.96 | 1.57 |
| RS7571570 | 3 | DTNB | 1838 | 2 | 25475034 | ‘C/T’ | A | 13.91 | 0.63 |
| RS9854395 | 3 | | | 3 | 47561518 | ‘A/G’ | A | 13.89 | 2.01 |
| RS959678 | 3 | ZPBP | 11055 | 7 | 50031156 | ‘A/G’ | A | 13.83 | 1.71 |
| RS16007 | 3 | CACNA1A | 773 | 19 | 13331316 | ‘A/G’ | A | 13.79 | 4.29 |
| RS2267064 | 3 | LOC648941 | 648941 | 22 | 22874632 | ‘G/T’ | C | 13.77 | 1.73 |
| RS873833 | 3 | CABIN1 | 23523 | 22 | 22757878 | ‘C/T’ | G | 13.77 | 1.73 |
| RS1159673 | 3 | | | 2 | 6623910 | ‘G/T’ | C | 13.75 | 1.71 |
| RS138981 | 3 | | | 22 | 41927759 | ‘A/G’ | A | 13.75 | 1.92 |
| RS4873814 | 3 | | | 8 | 144793335 | ‘A/G’ | G | 13.73 | 1.89 |
| RS1149907 | 3 | | | 10 | 8190108 | ‘A/G’ | A | 13.73 | 1.55 |
| RS1981736 | 3 | | | 2 | 66689823 | ‘A/G’ | A | 13.70 | 1.81 |
| RS2901483 | 3 | | | 2 | 62618776 | ‘A/G’ | A | 13.70 | 0.53 |
| RS4234091 | 3 | LOC651758 | 651758 | 2 | 241559700 | ‘A/G’ | A | 13.67 | 1.72 |
| RS7151518 | 3 | | | 14 | 101104559 | ‘A/G’ | A | 13.66 | 0.64 |
| RS7288568 | 3 | LOC648551 | 648551 | 22 | 47595366 | ‘C/T’ | A | 13.62 | 0.59 |
| RS3020835 | 3 | | | 7 | 141681285 | ‘A/C’ | C | 13.61 | 1.54 |
| RS4611181 | 3 | ZNF195 | 7748 | 11 | 3349321 | ‘C/T’ | G | 13.48 | 1.75 |
| RS4688807 | 3 | PLXND1 | 23129 | 3 | 130791953 | ‘C/T’ | A | 13.46 | 0.62 |
| RS861077 | 3 | | | 2 | 238992522 | ‘A/C’ | C | 13.43 | 1.62 |
| RS425246 | 3 | PLD5 | 200150 | 1 | 240470883 | ‘A/G’ | A | 13.41 | 0.47 |
| RS9506903 | 3 | | | 13 | 22150146 | ‘C/T’ | G | 13.39 | 0.63 |
| RS10492602 | 2 | | | 13 | 57737145 | ‘G/T’ | C | 13.37 | 3.70 |
| RS2152066 | 3 | TEK | 7010 | 9 | 27178862 | ‘G/T’ | A | 13.36 | 0.63 |
| RS1374868 | 3 | | | 3 | 74781680 | ‘A/G’ | A | 13.32 | 1.98 |
| RS8044769 | 3 | FTO | 79068 | 16 | 52396636 | ‘C/T’ | A | 13.30 | 0.65 |
| RS2391671 | 3 | CREB5 | 9586 | 7 | 28518902 | ‘A/G’ | G | 13.29 | 1.63 |
| RS499899 | 3 | | | 6 | 20109726 | ‘C/T’ | A | 13.28 | 1.61 |
| RS1891999 | 3 | | | 9 | 137226410 | ‘G/T’ | A | 13.25 | 1.58 |
| RS13054531 | 3 | | | 22 | 46392057 | ‘A/C’ | A | 13.22 | 0.32 |
| RS4651073 | 3 | XPR1 | 9213 | 1 | 178867222 | ‘A/G’ | G | 13.22 | 0.64 |
| RS1384634 | 3 | ZPBP | 11055 | 7 | 50035023 | ‘C/T’ | G | 13.20 | 1.54 |
| RS6904723 | 3 | BTBD9 | 114781 | 6 | 38544295 | ‘A/C’ | A | 13.20 | 1.53 |
| RS4294708 | 3 | | | 13 | 84403995 | ‘C/T’ | A | 13.15 | 1.65 |
| RS6538861 | 2 | | | 12 | 97274234 | ‘C/T’ | G | 13.15 | 1.88 |
| RS11911479 | 3 | | | 21 | 18463191 | ‘C/T’ | G | 13.04 | 0.54 |
| RS3850701 | 3 | | | 21 | 41988902 | ‘A/C’ | A | 13.03 | 0.30 |
| RS310025 | 3 | | | 16 | 79980481 | ‘A/G’ | A | 12.88 | 0.62 |
| RS6892814 | 3 | STK10 | 6793 | 5 | 171502017 | ‘A/G’ | A | 12.88 | 0.64 |
| RS1833036 | 3 | ESRRG | 2104 | 1 | 214773635 | ‘A/C’ | C | 12.85 | 1.53 |
| RS2837713 | 3 | DSCAM | 1826 | 21 | 40873626 | ‘A/C’ | A | 12.77 | 1.52 |
| RS3898917 | 3 | | | 11 | 5284937 | ‘G/T’ | C | 12.77 | 0.62 |
| RS11685593 | 3 | | | 2 | 127604591 | ‘C/T’ | A | 12.76 | 1.76 |
| RS6028637 | 3 | | | 20 | 37816039 | ‘C/T’ | A | 12.75 | 2.19 |
| RS10507024 | 3 | LOC651534 | 651534 | 12 | 91935819 | ‘A/G’ | A | 12.75 | 2.37 |
| RS2837709 | 3 | DSCAM | 1826 | 21 | 40861610 | ‘A/C’ | A | 12.73 | 1.52 |
| RS3848521 | 3 | | | 19 | 62264525 | ‘A/G’ | G | 12.67 | 1.62 |
| R81812315 | 3 | | | 15 | 25522899 | ‘C/T’ | G | 12.67 | 1.77 |
| RS1874622 | 3 | CRISPLD2 | 83716 | 16 | 83455234 | ‘A/G’ | G | 12.62 | 0.50 |
| RS6803083 | 3 | | | 3 | 79852274 | ‘G/T’ | C | 12.61 | 0.65 |
| RS1399333 | 3 | | | 4 | 10878977 | ‘C/T’ | G | 12.57 | 1.66 |
| RS17254891 | 3 | | | 3 | 61419483 | ‘C/T’ | G | 12.55 | 1.75 |
| RS3751812 | 3 | FTO | 79068 | 16 | 52375961 | ‘G/T’ | A | 12.54 | 1.52 |
| RS10876351 | 3 | | | 12 | 51482966 | ‘A/G’ | G | 12.54 | 1.60 |
| RS9591885 | 3 | | | 13 | 57981372 | ‘C/T’ | G | 12.53 | 3.58 |
| RS2671689 | 3 | | | 17 | 44918120 | ‘C/T’ | G | 12.53 | 1.98 |
| RS8050136 | 3 | FTO | 79068 | 16 | 52373776 | ‘A/C’ | A | 12.51 | 1.52 |
| RS199694 | 3 | ST6GALNAC5 | 81849 | 1 | 77276843 | ‘A/G’ | G | 12.49 | 1.96 |
| RS4399918 | 3 | NLGN1 | 22871 | 3 | 175087027 | ‘C/T’ | G | 12.48 | 0.50 |
| RS936960 | 3 | LIPC | 3990 | 15 | 56539169 | ‘A/C’ | A | 12.46 | 0.43 |
| RS3827256 | 3 | PFKL | 5211 | 21 | 44565251 | ‘A/G’ | A | 12.43 | 1.53 |
| RS17329247 | 2 | CHL1 | 10752 | 3 | 216913 | ‘A/G’ | A | 12.43 | 1.51 |
| RS1425531 | 3 | | | 4 | 143645481 | ‘C/T’ | A | 12.40 | 0.62 |
| RS2007215 | 3 | PTCND2 | 57540 | 1 | 11460564 | ‘A/G’ | A | 12.39 | 0.66 |
| RS2837716 | 3 | DSCAM | 1826 | 21 | 40875564 | ‘A/G’ | A | 12.36 | 1.51 |
| RS1022790 | 3 | | | 20 | 10678759 | ‘A/G’ | G | 12.33 | 1.51 |
| RS632912 | 2 | | | 18 | 8457707 | ‘A/G’ | A | 12.29 | 2.44 |
| RS2748173 | 3 | BTBD9 | 114781 | 6 | 38638120 | ‘A/G’ | G | 12.28 | 1.61 |
| RS711129 | 3 | | | 12 | 76568088 | ‘C/T’ | G | 12.26 | 0.53 |
| RS4735183 | 3 | | | 8 | 93638119 | ‘G/T’ | C | 12.24 | 0.63 |
| RS11733672 | 3 | | | 4 | 4653806 | ‘C/T’ | A | 12.22 | 1.67 |
| RS2824669 | 2 | | | 21 | 18457462 | ‘A/C’ | A | 12.20 | 0.65 |
| RS1605438 | 3 | | | 8 | 132605749 | ‘A/G’ | G | 12.20 | 0.66 |
| RS10513838 | 3 | | | 3 | 190715335 | ‘A/G’ | G | 12.19 | 2.27 |
| RS9614576 | 3 | ARHGAP8 | 23779 | 22 | 43603961 | ‘C/T’ | G | 12.16 | 0.66 |
| RS493524 | 3 | LOC651344 | 651344 | 11 | 78758254 | ‘C/T’ | A | 12.16 | 0.58 |
| RS4865755 | 3 | ITGA2 | 3673 | 5 | 52326944 | ‘C/T’ | G | 12.11 | 0.63 |
| RS4788480 | 3 | ATBF1 | 463 | 16 | 71493187 | ‘C/T’ | A | 12.11 | 1.60 |
| RS2369146 | 3 | | | 1 | 157934819 | ‘A/G’ | A | 12.10 | 0.59 |
| RS1093304 | 3 | KSR2 | 283455 | 12 | 116414534 | ‘C/T’ | A | 12.07 | 1.66 |
| RS462769 | 3 | MGC26885 | 124044 | 16 | 88290764 | ‘A/G’ | A | 12.07 | 1.51 |
| RS10962917 | 3 | | | 9 | 17220086 | ‘A/G’ | A | 12.06 | 0.57 |
| RS1495942 | 3 | | | 1 | 243142007 | ‘A/C’ | A | 12.06 | 2.24 |
| RS220836 | 3 | IGSF4 | 23705 | 11 | 114807081 | ‘A/G’ | G | 12.05 | 1.58 |
| RS10511820 | 1 | LRRN6C | 158038 | 9 | 28045603 | ‘A/C’ | C | 12.03 | 0.65 |
| RS983789 | 3 | | | 1 | 157862306 | ‘G/T’ | A | 12.02 | 1.91 |
| RS2864474 | 3 | TMEM105 | 284186 | 17 | 76916744 | ‘A/G’ | G | 12.01 | 0.61 |
| RS7630843 | 2 | | | 3 | 198681 | ‘C/T’ | G | 12.01 | 1.58 |
| RS3813587 | 3 | | | 19 | 22591133 | ‘A/C’ | C | 12.00 | 1.61 |
| RS2249963 | 3 | | | 8 | 11512635 | ‘C/T’ | G | 12.00 | 0.66 |
| RS1955716 | 3 | FBXO33 | 254170 | 14 | 38969200 | ‘C/T’ | G | 11.99 | 0.60 |
| RS1038853 | 3 | | | 4 | 108397270 | ‘A/C’ | C | 11.98 | 1.50 |
| RS4809656 | 3 | | | 20 | 45903393 | ‘G/T’ | C | 11.98 | 2.32 |
| RS2360090 | 3 | | | 2 | 195053028 | ‘A/G’ | G | 11.96 | 1.74 |
| RS6974985 | 3 | | | 7 | 149034703 | ‘C/T’ | A | 11.96 | 0.58 |
| RS9695286 | 3 | | | 9 | 109812888 | ‘C/T’ | A | 11.94 | 1.50 |
| RS6604634 | 3 | ESRRG | 2104 | 1 | 214787878 | ‘A/G’ | A | 11.93 | 1.50 |
| RS6989616 | 3 | ST18 | 9705 | 8 | 53272861 | ‘G/T’ | A | 11.92 | 0.36 |
| RS2166512 | 3 | | | 2 | 176779427 | ‘A/G’ | G | 11.92 | 1.52 |
| RS2785910 | 3 | | | 6 | 96439227 | ‘C/T’ | G | 11.92 | 1.52 |
| RS252682 | 3 | | | 5 | 106702415 | ‘A/G’ | G | 11.91 | 0.64 |
| RS1556867 | 3 | | | 1 | 162480310 | ‘C/T’ | A | 11.91 | 1.67 |
| RS6778227 | 3 | | | 3 | 182516536 | ‘C/T’ | G | 11.90 | 1.51 |
| RS1579303 | 3 | | | 5 | 180427946 | ‘A/G’ | A | 11.90 | 0.61 |
| RS7802349 | 3 | | | 7 | 85105110 | ‘C/T’ | G | 11.87 | 0.38 |
| RS11138526 | 2 | | | 9 | 81965277 | ‘G/T’ | A | 11.84 | 2.16 |
| RS2322606 | 3 | PTK2B | 2185 | 8 | 27242840 | ‘A/G’ | A | 11.84 | 1.68 |
| RS7501939 | 3 | TCF2 | 6928 | 17 | 33175269 | ‘C/T’ | A | 11.84 | 1.52 |
| RS4922157 | 3 | | | 8 | 20206493 | ‘C/T’ | G | 11.83 | 1.52 |
| RS6923737 | 3 | BTBD9 | 114781 | 6 | 38591542 | ‘C/T’ | G | 11.83 | 0.66 |
| RS9309828 | 3 | | | 3 | 79843464 | ‘A/G’ | A | 11.82 | 0.63 |
| RS6746082 | 3 | DTNB | 1838 | 2 | 25512748 | ‘A/C’ | C | 11.82 | 0.62 |
| RS5026446 | 3 | | | 18 | 74163927 | ‘C/T’ | A | 11.80 | 0.48 |
| RS7203175 | 3 | CDH13 | 1012 | 16 | 82261890 | ‘C/T’ | G | 11.80 | 2.10 |
| RS10245474 | 3 | ATXN7L4 | 222255 | 7 | 105249944 | ‘A/C’ | A | 11.79 | 0.62 |
| RS7613818 | 3 | CAST1 | 26059 | 3 | 55995199 | ‘A/G’ | G | 11.78 | 0.67 |
| RS9690428 | 3 | | | 7 | 52755728 | ‘C/T’ | G | 11.77 | 0.65 |
| RS956037 | 3 | TRAV16 | 28667 | 14 | 21528535 | ‘C/T’ | G | 11.73 | 0.21 |
| RS199689 | 3 | ST6GALNAC5 | 81849 | 1 | 77267267 | ‘A/C’ | C | 11.72 | 1.57 |
| RS7008482 | 3 | C8orf36 | 286053 | 8 | 126336812 | ‘G/T’ | C | 11.71 | 1.52 |
| RS10215277 | 3 | LOC641864 | 641864 | 7 | 141492186 | ‘C/T’ | A | 11.71 | 0.62 |
| RS12120303 | 3 | | | 1 | 66742956 | ‘A/G’ | A | 11.69 | 0.54 |
| RS12128593 | 3 | | | 1 | 66747467 | ‘C/T’ | G | 11.69 | 0.54 |
| RS6826645 | 3 | | | 4 | 44959803 | ‘A/G’ | A | 11.69 | 1.56 |
| RS10187702 | 3 | LOC652214 | 652214 | 2 | 58723279 | ‘C/T’ | G | 11.67 | 1.71 |
| RS12534779 | 3 | | | 7 | 135472243 | ‘A/G’ | G | 11.65 | 1.50 |
| RS936495 | 3 | | | 6 | 89209198 | ‘A/C’ | C | 11.61 | 0.51 |
| RS2267796 | 3 | GRIN2A | 2903 | 16 | 9884214 | ‘G/T’ | C | 11.58 | 0.62 |
| RS2607605 | 3 | | | 8 | 24700639 | ‘C/T’ | G | 11.57 | 1.73 |
| RS4945348 | 3 | LOC651344 | 651344 | 11 | 78797081 | ‘C/T’ | G | 11.57 | 0.54 |
| RS165774 | 3 | COMT | 1312 | 22 | 18332561 | ‘A/G’ | A | 11.56 | 0.63 |
| RS11993467 | 3 | PSD3 | 23362 | 8 | 18701941 | ‘C/T’ | A | 11.56 | 1.49 |
| RS2838818 | 3 | ADARB1 | 104 | 21 | 45465445 | ‘A/G’ | G | 11.55 | 1.49 |
| RS9686666 | 3 | | | 5 | 23398686 | ‘A/G’ | G | 11.54 | 1.49 |
| RS3787011 | 3 | RNF126 | 55658 | 19 | 612080 | ‘A/G’ | A | 11.54 | 2.47 |
| RS6447440 | 3 | | | 4 | 44955705 | ‘C/T’ | G | 11.54 | 1.55 |
| RS2167644 | 3 | LRRN6C | 158038 | 9 | 28076344 | ‘A/G’ | G | 11.52 | 0.62 |
| RS208003 | 3 | | | 7 | 19821959 | ‘C/T’ | A | 11.52 | 0.60 |
| RS7771891 | 3 | C6orf195 | 154386 | 6 | 2570303 | ‘A/G’ | G | 11.52 | 1.66 |
| RS2794515 | 3 | | | 1 | 157913168 | ‘A/G’ | A | 11.52 | 0.62 |
| RS9363388 | 3 | | | 6 | 66308721 | ‘C/T’ | G | 11.51 | 0.63 |
| RS3847437 | 3 | | | 10 | 11492266 | ‘C/T’ | A | 11.51 | 2.56 |
| RS7333943 | 2 | | | 13 | 58515848 | ‘G/T’ | C | 11.51 | 1.77 |
| RS11794056 | 3 | SNX30 | 401548 | 9 | 114644464 | ‘A/G’ | G | 11.51 | 0.63 |
| RS3760578 | 3 | SLC14A1 | 6563 | 18 | 41556974 | ‘A/G’ | A | 11.48 | 1.49 |
| RS11940185 | 3 | | | 4 | 19506456 | ‘A/G’ | A | 11.48 | 1.49 |
| RS3748971 | 3 | ECEL1P2 | 347694 | 2 | 232958927 | ‘A/G’ | A | 11.44 | 2.08 |
| RS1005142 | 3 | SV2B | 9899 | 15 | 89585011 | ‘C/T’ | G | 11.42 | 2.61 |
| RS10864069 | 3 | | | 1 | 212013892 | ‘A/C’ | A | 11.41 | 1.50 |
| RS220860 | 3 | IGSF4 | 23705 | 11 | 114799274 | ‘A/C’ | C | 11.41 | 1.60 |
| RS1515441 | 3 | SPATA16 | 83893 | 3 | 174317984 | ‘A/G’ | A | 11.40 | 2.09 |
| RS7335330 | 3 | LHFP | 10186 | 13 | 38905199 | ‘C/T’ | A | 11.40 | 1.69 |
| RS11919819 | 3 | SPATA16 | 83893 | 3 | 174315997 | ‘G/T’ | A | 11.40 | 1.93 |
| RS707896 | 3 | | | 6 | 26224403 | ‘A/G’ | A | 11.40 | 0.57 |
| RS6016142 | 3 | | | 20 | 37734221 | ‘C/T’ | A | 11.40 | 2.04 |
| RS1670533 | 3 | LOC285498 | 285498 | 4 | 1068187 | ‘C/T’ | G | 11.37 | 1.65 |
| RS12650866 | 3 | | | 4 | 84514285 | ‘A/C’ | A | 11.37 | 0.59 |
| RS7651591 | 3 | GRM7 | 2917 | 3 | 6898647 | ‘C/T’ | G | 11.36 | 1.98 |
| RS2655074 | 3 | | | 11 | 11157434 | ‘G/T’ | A | 11.36 | 1.51 |
| RS5768405 | 3 | | | 22 | 46941844 | ‘C/T’ | G | 11.33 | 1.53 |
| RS10899922 | 3 | C10orf136 | 414260 | 10 | 43661970 | ‘A/G’ | G | 11.30 | 1.51 |
| RS509063 | 3 | TRAF3IP1 | 26146 | 2 | 238960439 | ‘C/T’ | A | 11.30 | 1.64 |
| RS1123003 | 3 | | | 4 | 141658315 | ‘C/T’ | G | 11.29 | 1.70 |
| RS7791608 | 3 | KIAA1862 | 84626 | 7 | 149052706 | ‘A/G’ | G | 11.29 | 0.52 |
| RS767460 | 3 | CNTN4 | 152330 | 3 | 2716787 | ‘A/G’ | G | 11.29 | 0.63 |
| RS12615237 | 3 | | | 2 | 44131231 | ‘C/T’ | G | 11.28 | 0.59 |
| RS6831180 | 3 | | | 4 | 84523758 | ‘C/T’ | A | 11.27 | 0.60 |
| RS2897074 | 3 | | | 4 | 155157135 | ‘A/C’ | A | 11.26 | 1.49 |
| RS2283458 | 3 | SLCO3A1 | 28232 | 15 | 90490116 | ‘C/T’ | A | 11.26 | 0.67 |
| RS6586906 | 3 | | | 8 | 20222955 | ‘A/G’ | G | 11.25 | 1.57 |
| RS3822292 | 3 | TACR3 | 6870 | 4 | 104776161 | ‘A/G’ | G | 11.24 | 1.92 |
| RS10485483 | 3 | CDS2 | 8760 | 20 | 5106354 | ‘A/C’ | A | 11.24 | 0.51 |
| RS1411850 | 3 | | | 6 | 66305145 | ‘C/T’ | G | 11.24 | 0.63 |
| RS8051575 | 3 | GAN | 8139 | 16 | 79958316 | ‘G/T’ | A | 11.24 | 1.48 |
| RS532040 | 3 | | | 11 | 129081186 | ‘C/T’ | A | 11.23 | 1.64 |
| RS10922232 | 3 | | | 1 | 187789366 | ‘G/T’ | C | 11.22 | 1.48 |
| RS10495029 | 3 | ESRRG | 2104 | 1 | 214792606 | ‘C/T’ | G | 11.21 | 0.66 |
| RS1510510 | 3 | | | 2 | 239164074 | ‘G/T’ | A | 11.21 | 1.89 |
| RS1476880 | 3 | | | 4 | 24345356 | ‘G/T’ | A | 11.21 | 1.49 |
| RS3760352 | 3 | ASGR2 | 433 | 17 | 6960602 | ‘C/T’ | G | 11.20 | 0.64 |
| RS1870943 | 3 | | | 12 | 88192283 | ‘C/T’ | G | 11.20 | 2.06 |
| RS6550169 | 3 | LOC651301 | 651301 | 3 | 32888097 | ‘C/T’ | A | 11.19 | 1.48 |
| RS7770868 | 3 | BTBD9 | 114781 | 6 | 38572604 | ‘A/C’ | A | 11.18 | 1.48 |
| RS977576 | 3 | | | 5 | 52592967 | ‘C/T’ | G | 11.17 | 1.48 |
| RS6980380 | 3 | PRKAG2 | 51422 | 7 | 151018453 | ‘C/T’ | G | 11.17 | 1.71 |
| RS11768400 | 3 | | | 7 | 84893471 | ‘A/G’ | G | 11.17 | 0.67 |
| RS10773557 | 3 | | | 12 | 127638497 | ‘A/C’ | A | 11.16 | 0.67 |
| RS6136703 | 3 | SLC24A3 | 57419 | 20 | 19320443 | ‘C/T’ | A | 11.15 | 2.23 |
| RS2001902 | 2 | | | 22 | 46948268 | ‘A/C’ | C | 11.15 | 1.55 |
| RS1986437 | 3 | | | 12 | 81067405 | ‘A/G’ | A | 11.14 | 0.61 |
| RS10484432 | 3 | | | 6 | 26116855 | ‘A/G’ | A | 11.14 | 0.61 |
| RS10223320 | 3 | | | 5 | 158506964 | ‘C/T’ | G | 11.14 | 1.48 |
| RS2291347 | 3 | ADAMTS8 | 11095 | 11 | 129791976 | ‘A/G’ | G | 11.13 | 1.48 |
| RS11665875 | 3 | | | 19 | 6951401 | ‘C/T’ | G | 11.12 | 1.48 |
| RS7923262 | 3 | | | 10 | 71895542 | ‘A/G’ | G | 11.12 | 0.66 |
| RS3858054 | 3 | | | 9 | 8243589 | ‘C/T’ | G | 11.11 | 1.49 |
| RS459920 | 3 | C16orf55 | 124045 | 16 | 88258328 | ‘C/T’ | A | 11.11 | 1.48 |
| RS6872241 | 3 | | | 5 | 151061730 | ‘C/T’ | G | 11.10 | 1.56 |
| RS13105217 | 3 | | | 4 | 65064629 | ‘C/T’ | G | 11.10 | 0.64 |
| RS10513039 | 3 | | | 5 | 9974028 | ‘A/G’ | A | 11.10 | 1.56 |
| RS2492624 | 3 | | | 9 | 29928824 | ‘A/G’ | G | 11.09 | 0.66 |
| RS4547623 | 3 | | | 22 | 36322650 | ‘A/G’ | A | 11.08 | 1.50 |
| RS17862309 | 3 | GRM8 | 2918 | 7 | 126598442 | ‘C/T’ | G | 11.07 | 0.12 |
| RS138957 | 3 | KIAA0153 | 23170 | 22 | 41914173 | ‘G/T’ | C | 11.06 | 1.64 |
| RS4145462 | 3 | MPZL1 | 9019 | 1 | 165985123 | ‘A/G’ | A | 11.06 | 0.12 |
| RS9506776 | 3 | LOC650912 | 650912 | 13 | 21518850 | ‘C/T’ | A | 11.05 | 1.52 |
| RS1926324 | 3 | DCAMKL1 | 9201 | 13 | 35582443 | ‘C/T’ | A | 11.03 | 1.57 |
| RS2269903 | 3 | CHN2 | 1124 | 7 | 29213934 | ‘A/C’ | C | 11.02 | 1.99 |
| RS410202 | 3 | | | 4 | 44946963 | ‘C/T’ | G | 11.01 | 1.54 |
| RS2560623 | 3 | | | 5 | 116742235 | ‘A/G’ | A | 11.01 | 0.48 |
| RS1037973 | 3 | GAN | 8139 | 16 | 79957577 | ‘C/T’ | A | 11.00 | 0.67 |
| RS12038863 | 3 | | | 1 | 178843066 | ‘A/C’ | C | 11.00 | 0.49 |
| R82934477 | 3 | CRISPLD2 | 83716 | 16 | 83480748 | ‘A/C’ | A | 10.97 | 0.67 |
| RS9558678 | 3 | | | 13 | 105554332 | ‘C/T’ | G | 10.95 | 0.56 |
| RS1327904 | 3 | C20orf26 | 26074 | 20 | 20168568 | ‘A/C’ | C | 10.94 | 1.51 |
| RS2458291 | 3 | ATP6V1C1 | 528 | 8 | 104138327 | ‘C/T’ | A | 10.94 | 1.67 |
| RS181246 | 3 | | | 17 | 53561087 | ‘G/T’ | A | 10.93 | 0.64 |
| RS1282129 | 3 | | | 1 | 111424262 | ‘A/G’ | G | 10.92 | 1.49 |
| RS7943619 | 3 | | | 11 | 105863808 | ‘C/T’ | G | 10.91 | 1.54 |
| RS2164498 | 3 | LOC649035 | 649035 | 12 | 31250477 | ‘A/G’ | A | 10.91 | 0.55 |
| RS9790415 | 3 | | | 4 | 141648328 | ‘A/G’ | G | 10.90 | 1.68 |
| RS1391619 | 3 | | | 11 | 5412505 | ‘C/T’ | G | 10.90 | 0.59 |
| RS1332339 | 3 | | | 9 | 25946931 | ‘A/G’ | G | 10.89 | 0.52 |
| RS12184120 | 3 | | | 7 | 92556975 | ‘A/G’ | A | 10.89 | 0.50 |
| RS3922855 | 3 | | | 15 | 24625657 | ‘C/T’ | G | 10.89 | 0.63 |
| RS911745452 | 3 | | | 5 | 83182789 | ‘C/T’ | A | 10.87 | 1.70 |
| RS12026602 | 3 | | | 1 | 11440984 | ‘C/T’ | G | 10.85 | 0.68 |
| RS950942 | 3 | | | 13 | 70429810 | ‘C/T’ | A | 10.85 | 1.48 |
| RS2045065 | 3 | LOC647947 | 647947 | 4 | 1042488 | ‘C/T’ | A | 10.85 | 1.63 |
| RS1439354 | 3 | | | 4 | 44296521 | ‘G/T’ | C | 10.85 | 0.64 |
| RS2299554 | 3 | GRM8 | 2918 | 7 | 126644390 | ‘C/T’ | G | 10.84 | 0.36 |
| RS2284218 | 3 | CRHR2 | 1395 | 7 | 30680858 | ‘C/T’ | G | 10.84 | 0.67 |
| RS1463342 | 3 | FLJ39822 | 151258 | 2 | 165520674 | ‘A/G’ | A | 10.83 | 0.61 |
| RS2447523 | 3 | | | 11 | 33418920 | ‘A/G’ | G | 10.83 | 0.61 |
| RS1206810 | 3 | EYA2 | 2139 | 20 | 45128769 | ‘C/T’ | A | 10.83 | 0.65 |
| RS13251222 | 3 | | | 8 | 79207857 | ‘A/G’ | G | 10.83 | 0.63 |
| RS5583190 | 3 | CACNA2D3 | 55799 | 3 | 54702117 | ‘A/C’ | C | 10.83 | 1.47 |
| RS1331205 | 3 | | | 6 | 66326120 | ‘A/G’ | G | 10.83 | 0.64 |
| RS1264215 | 3 | HEPH | 9843 | X | 65337334 | ‘C/T’ | A | 12.22 | 0.00 |
| RS5845127 | 2 | | | X | 7785325 | ‘A/G’ | A | 16.73 | 0.38 |
| RS5955922 | 3 | | | X | 17970012 | ‘C/T’ | G | 15.78 | 2.36 |
| RS3788776 | 3 | ODZ1 | 10178 | X | 123512044 | ‘A/G’ | G | 15.96 | 2.25 |
| RS2178544 | 3 | | | X | 7934954 | ‘G/T’ | C | 11.41 | 0.52 |
| RS6527728 | 3 | CXorf15 | 55787 | X | 16729886 | ‘A/G’ | A | 23.11 | 2.79 |
| RS6522746 | 3 | | | X | 93236536 | ‘C/T’ | A | 14.02 | 2.10 |
| RS596987 | 3 | | | X | 144193420 | ‘A/G’ | G | 11.43 | 0.55 |
| RS995895 | 3 | | | X | 144258291 | ‘A/G’ | A | 14.58 | 0.52 |
| RS5905817 | 3 | | | X | 44180283 | ‘A/C’ | A | 14.25 | 0.51 |
| RS7063947 | 3 | | | X | 141196819 | ‘A/G’ | A | 11.02 | 1.65 |
| RS4898198 | 3 | | | X | 24977352 | ‘A/C’ | C | 11.37 | 0.61 |
| RS1531812 | 3 | | | X | 5712016 | ‘C/T’ | A | 16.62 | 0.54 |
| RS5986723 | 3 | | | X | 24965806 | ‘A/G’ | G | 11.34 | 0.61 |
| RS5961851 | 3 | | | X | 5722793 | ‘A/G’ | G | 18.56 | 1.85 |
| RS11091940 | 3 | | | X | 93198011 | ‘A/G’ | G | 12.59 | 1.64 |
| RS5970648 | 3 | | | X | 22750361 | ‘C/T’ | G | 11.45 | 1.56 |
| RS1458368 | 3 | DMD | 1756 | X | 31730435 | ‘A/G’ | A | 10.93 | 1.57 |
| RS5936438 | 3 | AFF2 | 2334 | X | 147694315 | ‘A/G’ | A | 12.74 | 0.62 |
| RS1361680 | 3 | | | X | 93217606 | ‘A/G’ | A | 13.32 | 1.62 |
| RS5983336 | 3 | | | X | 93209681 | ‘A/C’ | C | 12.98 | 1.61 |
| RS10522062 | 3 | | | X | 93207413 | ‘A/G’ | C | 12.75 | 1.60 |
| RS4503212 | 2 | RGN | 9104 | X | 46826402 | ‘A/G’ | G | 17.15 | 0.58 |
| RS3850163 | 3 | | | X | 28355558 | ‘G/T’ | C | 12.06 | 1.57 |
| RS3863537 | 3 | | | X | 13030051 | ‘C/T’ | G | 11.18 | 0.65 |
| RS5962469 | 3 | IL1RAPL2 | 26280 | X | 104285901 | ‘A/G’ | A | 13.36 | 0.63 |
| RS6616567 | 3 | IL1RAPL2 | 26280 | X | 104244418 | ‘C/T’ | A | 12.44 | 0.64 |
| RS5953334 | 3 | LOC139163 | 139163 | X | 49330138 | ‘C/T’ | C | 12.44 | 0.64 |
| RS5931268 | 2 | | | X | 136893265 | ‘G/T’ | A | 13.33 | 0.64 |
| RS5961861 | 3 | | | X | 5738176 | ‘C/T’ | G | 16.23 | 1.64 |
| RS731426 | 3 | CXorf6 | 10046 | X | 149395757 | ‘C/T’ | A | 13.01 | 1.56 |
| RS5905269 | 2 | | | X | 115402180 | ‘A/C’ | A | 10.89 | 0.67 |
| RS2366513 | 2 | | | X | 136878343 | ‘A/G’ | G | 17.83 | 1.67 |
| RS2366517 | 2 | | | X | 136887785 | ‘C/T’ | G | 17.47 | 1.66 |
| RS5918294 | 3 | | | X | 41854429 | ‘C/T’ | A | 19.01 | 1.70 |
| RS1007490 | 3 | | | X | 22745174 | ‘C/T’ | G | 11.19 | 1.50 |
| RS2886700 | 2 | | | X | 136849214 | ‘C/T’ | G | 18.51 | 1.68 |
| RS1293468 | 3 | | | X | 122036209 | ‘C/T’ | A | 11.87 | 0.66 |
| RS1293545 | 3 | | | X | 121956842 | ‘A/G’ | G | 11.85 | 0.66 |
| RS5935799 | 3 | GLRA2 | 2742 | X | 14641184 | ‘A/G’ | A | 11.44 | 1.50 |
| RS909659 | 3 | | | X | 143894286 | ‘C/T’ | G | 11.98 | 1.51 |
| RS2128519 | 3 | | | X | 5626964 | ‘C/T’ | G | 10.91 | 1.48 |
| RS2269584 | 3 | PPEF1 | 5475 | X | 18689642 | ‘A/G’ | A | 10.98 | 0.68 |
| RS4825236 | 3 | PPEF1 | 5475 | X | 18642674 | ‘C/T’ | G | 10.87 | 0.68 |
| RS4825420 | 3 | | | X | 116062304 | ‘G/T’ | A | 10.83 | 1.48 |
| RS7059239 | 3 | PPEF1 | 5475 | X | 18622368 | ‘A/G’ | A | 11.49 | 1.49 |
| RS5909201 | 3 | PPEF1 | 5475 | X | 18623637 | ‘C/T’ | A | 11.49 | 1.49 |

dbSNP_rs_ID: SNP identification number in NCBI dbSNP database
Gene_locus: Gene locus and gene id as reported by NCBI dbSNP database build 126
Priority_date: SNP listed in 1: US 11/245,248 2: US 60/819,014 3: US 60/867,454
Sequence_ID: Sequence identification number
Position: Basepair Position, SNP physical position according to NCBI Human Genome Build 36.1
Variants: Alternate SNP alleles or their complementary nucleotides in the position indicated by dbSNP RS ID and basepair position
Minor Allele: SNP allele or its complementary nucleotide that is less common in the control population.
Allele_X2: Chi-squared test based on allele frequencies
Odds ratio: Calculated for the minor allele.
Gene_content: Genes positioned within 100 Kbp up and downstream from the physical position of the SNPs based on NCBI Human Genome Build 36.

TABLE 6
SNP markers with the strongest association with hypertension in the regression analysis with an
additive genotype model and T2D as a covariate. The analysis is based on the combined data of 110 HT cases
and 110 healthy controls from the Ashkenazi Jew population, 114 HT cases and 114 healthy controls from
the East Finnish population, 41 HT cases and 41 healthy controls from the German population and 28 HT
cases and 28 healthy controls from the English population.

| | | Gene locus | | | | | | |
|---|---|---|---|---|---|---|---|---|
| | | and | | | | | | |
| dbSNP rs ID | Priority date | Gene ID | | Chromosome | Position | Variats | Coefficient | P value |
| RS2245192 | 3 | | | 7 | 113789771 | ‘C/T’ | −0.83 | 1.23E−05 |
| RS2458291 | 3 | ATP6V1C1 | 528 | 8 | 104138327 | ‘C/T’ | −0.81 | 2.01E−05 |
| RS7406978 | 3 | ABR | 29 | 17 | 983909 | ‘C/T’ | −0.58 | 2.06E−05 |
| RS934083 | 3 | CACNA2D3 | 55799 | 3 | 54765419 | ‘C/T’ | 0.57 | 2.64E−05 |
| RS11088668 | 3 | | | 21 | 18448920 | ‘C/T’ | −0.84 | 3.24E−05 |
| RS2256182 | 3 | | | 8 | 93637241 | ‘C/T’ | 0.69 | 4.09E−05 |
| RS6474169 | 3 | ADAM18 | 8749 | 8 | 39697120 | ‘G/T’ | 0.58 | 5.24E−05 |
| RS711129 | 3 | | | 12 | 76568088 | ‘C/T’ | −0.89 | 5.85E−05 |
| RS4891635 | 3 | | | 18 | 63741526 | ‘C/T’ | 0.99 | 6.71E−05 |
| RS261988 | 3 | | | 5 | 95866641 | ‘A/G’ | 0.57 | 6.75E−05 |
| RS165774 | 3 | COMT | 1312 | 22 | 18332561 | ‘A/G’ | 0.61 | 7.53E−05 |
| RS1369704 | 3 | DTNB | 1838 | 2 | 25477094 | ‘A/G’ | −0.59 | 9.82E−05 |
| RS1022790 | 3 | | | 20 | 10678759 | ‘A/G’ | 0.55 | 1.07E−04 |
| RS189947 | 3 | | | 21 | 17556641 | ‘A/C’ | 0.58 | 1.17E−04 |
| RS6892814 | 3 | STK10 | 6793 | 5 | 171502017 | ‘A/G’ | 0.55 | 1.19E−04 |
| RS3900775 | 3 | SMOC2 | 64094 | 6 | 168613353 | ‘C/T’ | −0.74 | 1.28E−04 |
| RS11650418 | 3 | ABR | 29 | 17 | 1025021 | ‘G/T’ | 0.54 | 1.30E−04 |
| RS6444191 | 3 | ST6GAL1 | 6480 | 3 | 188182304 | ‘A/G’ | −0.55 | 1.32E−04 |
| RS7141 | 3 | EFNB3 | 1949 | 17 | 7555326 | ‘A/G’ | 0.57 | 1.40E−04 |
| RS6828802 | 3 | | | 4 | 292934 | ‘C/T’ | 0.56 | 1.47E−04 |
| RS2329727 | 3 | | | 7 | 51368212 | ‘A/G’ | 1.00 | 1.48E−04 |
| RS2901483 | 3 | | | 2 | 62618776 | ‘A/G’ | 0.78 | 1.55E−04 |
| RS290048 | 3 | | | 2 | 77381720 | ‘A/G’ | −1.45 | 1.69E−04 |
| RS17254891 | 3 | | | 3 | 61419483 | ‘C/T’ | 0.70 | 1.70E−04 |
| RS6872241 | 3 | | | 5 | 151061730 | ‘C/T’ | 0.62 | 1.72E−04 |
| RS1721355 | 3 | WDR69 | 164781 | 2 | 228491220 | ‘A/G’ | −0.52 | 1.72E−04 |
| RS1384634 | 3 | ZPBP | 11055 | 7 | 50035023 | ‘C/T’ | 0.52 | 1.74E−04 |
| RS1488547 | 3 | NLGN1 | 22871 | 3 | 175008462 | ‘C/T’ | 0.56 | 1.86E−04 |
| RS351211 | 3 | | | 15 | 72363918 | ‘G/T’ | −0.64 | 1.94E−04 |
| RS7648607 | 3 | MITF | 4286 | 3 | 69919626 | ‘A/C’ | −0.51 | 1.97E−04 |
| RS9564765 | 3 | | | 13 | 70431786 | ‘A/G’ | 0.56 | 1.99E−04 |
| RS17699211 | 3 | | | 12 | 3903752 | ‘C/T’ | 0.81 | 2.08E−04 |
| RS2447523 | 3 | | | 11 | 33418920 | ‘A/G’ | −0.64 | 2.28E−04 |
| RS4873814 | 3 | | | 8 | 144793335 | ‘A/G’ | 0.76 | 2.30E−04 |
| RS1901388 | 3 | ADAM18 | 8749 | 8 | 39672418 | ‘C/T’ | 0.53 | 2.35E−04 |
| RS7539199 | 3 | | | 1 | 34855853 | ‘A/G’ | 0.78 | 2.39E−04 |
| RS6980380 | 3 | PRKAG2 | 51422 | 7 | 151018453 | ‘C/T’ | 0.71 | 2.43E−04 |
| RS2249963 | 3 | | | 8 | 11512635 | ‘C/T’ | −0.50 | 2.50E−04 |
| RS7305776 | 3 | | | 12 | 76122239 | ‘A/G’ | 0.55 | 2.53E−04 |
| RS12140392 | 3 | | | 1 | 186420880 | ‘A/C’ | −0.61 | 2.55E−04 |
| RS6574791 | 3 | | | 14 | 19800970 | ‘C/T’ | −0.79 | 2.66E−04 |
| RS4143444 | 3 | | | 6 | 91103018 | ‘C/T’ | −0.83 | 2.67E−04 |
| RS13074723 | 3 | NLGN1 | 22871 | 3 | 175004791 | ‘A/G’ | −0.55 | 2.85E−04 |
| RS6466963 | 3 | LOC401398 | 401398 | 7 | 124359524 | ‘A/G’ | −0.53 | 2.89E−04 |
| RS9863894 | 3 | NLGN1 | 22871 | 3 | 174960398 | ‘C/T’ | 0.55 | 2.94E−04 |
| RS4246861 | 3 | | | 9 | 25589995 | ‘C/T’ | 0.62 | 2.96E−04 |
| RS2852217 | 3 | GRIK4 | 2900 | 11 | 120152436 | ‘C/T’ | 0.56 | 2.98E−04 |
| RS10833533 | 3 | | | 11 | 3214412 | ‘A/G’ | −0.65 | 3.04E−04 |
| RS2610725 | 3 | | | 6 | 89322376 | ‘C/T’ | −0.51 | 3.09E−04 |
| RS3130559 | 3 | PSORS1C1 | 170679 | 6 | 31205280 | ‘C/T’ | −0.56 | 3.16E−04 |
| RS583190 | 3 | CACNA2D3 | 55799 | 3 | 54702117 | ‘A/C’ | 0.47 | 3.24E−04 |
| RS9309828 | 3 | | | 3 | 79843464 | ‘A/G’ | 0.56 | 3.27E−04 |
| RS10519722 | 3 | LOC644055 | 644055 | 2 | 6298399 | ‘A/G’ | 0.50 | 3.27E−04 |
| RS2173086 | 3 | KIAA1040 | 23041 | 12 | 61165237 | ‘A/G’ | 0.70 | 3.28E−04 |
| RS10245474 | 3 | ATXN7L4 | 222255 | 7 | 105249944 | ‘A/C’ | 0.59 | 3.29E−04 |
| RS6604634 | 3 | ESRRG | 2104 | 1 | 214787878 | ‘A/G’ | −0.49 | 3.35E−04 |
| RS6128804 | 3 | | | 20 | 58403830 | ‘A/G’ | 0.62 | 3.47E−04 |
| RS11025056 | 3 | | | 11 | 19186741 | ‘A/G’ | −0.90 | 3.49E−04 |
| RS1874622 | 3 | CRISPLD2 | 83716 | 16 | 83455234 | ‘A/G’ | −0.83 | 3.50E−04 |
| RS1399333 | 3 | | | 4 | 10878977 | ‘C/T’ | 0.60 | 3.54E−04 |
| RS17523117 | 3 | | | 5 | 124691426 | ‘A/G’ | −0.52 | 3.54E−04 |
| RS10962917 | 3 | | | 9 | 17220086 | ‘A/G’ | 0.69 | 3.55E−04 |
| RS2723167 | 3 | | | 2 | 113337681 | ‘C/T’ | −0.48 | 3.56E−04 |
| RS13000621 | 3 | | | 2 | 181301492 | ‘A/G’ | 0.49 | 3.56E−04 |
| RS7318557 | 3 | LHFP | 10186 | 13 | 38818286 | ‘C/T’ | −0.54 | 3.58E−04 |
| RS1744493 | 3 | | | 6 | 165577651 | ‘C/T’ | −0.53 | 3.60E−04 |
| RS7601055 | 3 | KIAA1486 | 57624 | 2 | 226028326 | ‘C/T’ | −0.96 | 3.63E−04 |
| RS10506851 | 3 | PPFIA2 | 8499 | 12 | 80662290 | ‘A/G’ | −0.68 | 3.71E−04 |
| RS6074018 | 3 | MANBAL | 63905 | 20 | 35358710 | ‘A/G’ | −0.62 | 3.76E−04 |
| RS9572943 | 3 | | | 13 | 71751202 | ‘C/T’ | −0.72 | 3.78E−04 |
| RS6890771 | 3 | | | 5 | 180014372 | ‘C/T’ | 0.49 | 3.95E−04 |
| RS4688807 | 3 | PLXND1 | 23129 | 3 | 130791953 | ‘C/T’ | 0.54 | 4.05E−04 |
| RS7125888 | 3 | AMPD3 | 272 | 11 | 10466848 | ‘C/T’ | −0.50 | 4.09E−04 |
| RS8010116 | 3 | | | 14 | 19793535 | ‘C/T’ | −0.76 | 4.23E−04 |
| RS12631548 | 3 | CAST1 | 26059 | 3 | 56011031 | ‘C/T’ | −0.68 | 4.34E−04 |
| RS2861598 | 3 | NLGN1 | 22871 | 3 | 175027506 | ‘C/T’ | 0.52 | 4.35E−04 |
| RS2454043 | 3 | ATP6V1C1 | 528 | 8 | 104139431 | ‘G/T’ | 0.52 | 4.39E−04 |
| RS6436553 | 3 | KIAA1486 | 57624 | 2 | 226002129 | ‘A/G’ | −0.95 | 4.39E−04 |
| RS4865755 | 3 | ITGA2 | 3673 | 5 | 52326944 | ‘C/T’ | −0.57 | 4.40E−04 |
| RS6852347 | 3 | PPP3CA | 5530 | 4 | 102436590 | ‘C/T’ | 0.49 | 4.48E−04 |
| RS11911479 | 3 | | | 21 | 18463191 | ‘C/T’ | −0.72 | 4.50E−04 |
| RS6500316 | 3 | | | 16 | 49108242 | ‘A/G’ | −0.51 | 4.52E−04 |
| RS7440788 | 3 | | | 4 | 59837784 | ‘C/T’ | 0.71 | 4.53E−04 |
| RS1425531 | 3 | | | 4 | 143645481 | ‘C/T’ | 0.56 | 4.59E−04 |
| RS204505 | 3 | | | 9 | 117927770 | ‘C/T’ | −0.49 | 4.63E−04 |
| RS1838733 | 3 | PDE4D | 5144 | 5 | 58569149 | ‘A/G’ | −0.54 | 4.63E−04 |
| RS846491 | 3 | KATNAL1 | 84056 | 13 | 29743131 | ‘C/T’ | 0.81 | 4.70E−04 |
| RS2165857 | 3 | ABR | 29 | 17 | 954904 | ‘C/T’ | −0.49 | 4.72E−04 |
| RS697550 | 3 | | | 5 | 14125453 | ‘G/T’ | 0.61 | 4.79E−04 |
| RS12133017 | 3 | C1orf125 | 126859 | 1 | 177708960 | ‘C/T’ | 0.49 | 4.81E−04 |
| RS4447608 | 3 | | | 2 | 113322428 | ‘C/T’ | −0.47 | 4.84E−04 |
| RS2044961 | 3 | LRRN6C | 158038 | 9 | 28154645 | ‘A/G’ | −0.48 | 4.84E−04 |
| RS477558 | 3 | | | 1 | 18092414 | ‘A/G’ | −0.47 | 4.86E−04 |
| RS984923 | 3 | | | 8 | 24690660 | ‘A/G’ | 0.50 | 4.91E−04 |
| RS6882366 | 3 | | | 5 | 95890449 | ‘C/T’ | −0.51 | 4.91E−04 |
| RS4735183 | 3 | | | 8 | 93638119 | ‘G/T’ | −0.54 | 4.94E−04 |
| RS10872824 | 3 | | | 10 | 133356838 | ‘A/G’ | 0.60 | 4.96E−04 |
| RS1928863 | 3 | | | 9 | 12240170 | ‘A/G’ | −0.47 | 4.97E−04 |
| RS11791609 | 3 | | | 9 | 12246453 | ‘A/G’ | 0.48 | 4.97E−04 |
| RS2040859 | 3 | PIK3C2A | 5286 | 11 | 17104753 | ‘C/T’ | −0.56 | 5.00E−04 |
| RS17140205 | 3 | | | 5 | 115970039 | ‘A/G’ | −0.50 | 5.11E−04 |
| RS6595959 | 3 | LOC649922 | 649922 | 5 | 97938006 | ‘C/T’ | 0.65 | 5.15E−04 |
| RS767460 | 3 | CNTN4 | 152330 | 3 | 2716787 | ‘A/G’ | −0.55 | 5.25E−04 |
| RS6885761 | 3 | | | 5 | 154595473 | ‘A/G’ | 0.99 | 5.28E−04 |
| RS11685593 | 3 | | | 2 | 127604591 | ‘C/T’ | −0.61 | 5.31E−04 |
| RS6550169 | 3 | LOC651301 | 651301 | 3 | 32888097 | ‘C/T’ | −0.47 | 5.34E−04 |
| RS6926970 | 3 | ENPP1 | 5167 | 6 | 132208983 | ‘A/C’ | 0.66 | 5.35E−04 |
| RS827228 | 3 | ARHGAP15 | 55843 | 2 | 143626189 | ‘C/T’ | 0.48 | 5.38E−04 |
| RS1332855 | 3 | | | 9 | 82827607 | ‘G/T’ | −0.70 | 5.40E−04 |
| RS2934477 | 3 | CRISPLD2 | 83716 | 16 | 83480748 | ‘A/C’ | 0.47 | 5.44E−04 |
| RS8066575 | 3 | FLJ45831 | 400576 | 17 | 14631647 | ‘C/T’ | −0.53 | 5.45E−04 |
| RS6038092 | 3 | | | 20 | 5137472 | ‘A/G’ | −0.81 | 5.46E−04 |
| RS2225213 | 3 | C1orf125 | 126859 | 1 | 177725444 | ‘C/T’ | 0.48 | 5.48E−04 |
| RS2283379 | 3 | RGS6 | 9628 | 14 | 71998582 | ‘G/T’ | −0.62 | 5.55E−04 |
| RS1155847 | 3 | PRSS7 | 5651 | 21 | 18626167 | ‘C/T’ | −0.84 | 5.57E−04 |
| RS7008482 | 3 | C8orf36 | 286053 | 8 | 126336812 | ‘G/T’ | 0.48 | 5.60E−04 |
| RS12038863 | 3 | | | 1 | 178843066 | ‘A/C’ | −0.88 | 5.60E−04 |
| RS1971877 | 3 | | | 2 | 6292967 | ‘A/G’ | 0.54 | 5.64E−04 |
| RS7334289 | 3 | | | 13 | 37000990 | ‘C/T’ | 0.68 | 5.70E−04 |
| RS7335400 | 3 | | | 13 | 37001152 | ‘A/C’ | −0.68 | 5.70E−04 |
| RS1544452 | 3 | | | 7 | 124168925 | ‘A/G’ | −0.51 | 5.72E−04 |
| RS590218 | 3 | ZNF516 | 9658 | 18 | 72198871 | ‘A/G’ | −0.67 | 5.77E−04 |
| RS111524 | 3 | HSH2D | 84941 | 19 | 16126494 | ‘C/T’ | −0.57 | 5.78E−04 |
| RS12487554 | 3 | SEC22L2 | 26984 | 3 | 124425472 | ‘A/G’ | 0.49 | 5.85E−04 |
| RS1429376 | 3 | XDH | 7498 | 2 | 31442065 | ‘A/C’ | 0.58 | 5.87E−04 |
| RS1795502 | 3 | LIN7A | 8825 | 12 | 79791804 | ‘A/C’ | −0.63 | 5.88E−04 |
| RS1163665 | 3 | LIN7A | 8825 | 12 | 79813991 | ‘C/T’ | 0.63 | 5.88E−04 |
| RS2301301 | 3 | HOXD3 | 3232 | 2 | 176740513 | ‘C/T’ | 0.47 | 5.96E−04 |
| RS1383750 | 3 | LOC401398 | 401398 | 7 | 124488002 | ‘C/T’ | 0.48 | 5.98E−04 |
| RS827226 | 3 | ARHGAP15 | 55843 | 2 | 143639083 | ‘C/T’ | −0.47 | 6.00E−04 |
| RS7901709 | 3 | | | 10 | 110273057 | ‘C/T’ | −0.48 | 6.01E−04 |
| RS1833036 | 3 | ESRRG | 2104 | 1 | 214773635 | ‘A/C’ | 0.47 | 6.05E−04 |
| RS10743601 | 3 | STK38L | 23012 | 12 | 27300343 | ‘A/G’ | 0.60 | 6.10E−04 |
| RS4807030 | 3 | | | 19 | 5340941 | ‘A/G’ | 0.52 | 6.13E−04 |
| RS8063120 | 3 | | | 16 | 80352553 | ‘A/G’ | −0.49 | 6.16E−04 |
| RS1425533 | 3 | | | 4 | 143640625 | ‘C/T’ | 0.57 | 6.18E−04 |
| RS11199496 | 3 | | | 10 | 122439906 | ‘C/T’ | −0.51 | 6.18E−04 |
| RS2391671 | 3 | CREB5 | 9586 | 7 | 28518902 | ‘A/G’ | 0.52 | 6.19E−04 |
| RS2703833 | 3 | KCTD8 | 386617 | 4 | 44036287 | ‘C/T’ | 0.62 | 6.28E−04 |
| RS4277860 | 3 | | | 5 | 67511708 | ‘A/G’ | 0.73 | 6.29E−04 |
| RS11582225 | 3 | | | 1 | 162108767 | ‘C/T’ | −0.67 | 6.39E−04 |
| RS3863537 | 3 | | | X | 13030051 | ‘C/T’ | −0.43 | 6.39E−04 |
| RS6040345 | 3 | | | 20 | 11011477 | ‘A/G’ | 0.49 | 6.45E−04 |
| RS2025245 | 3 | | | 13 | 37001577 | ‘A/G’ | 0.67 | 6.45E−04 |
| RS2322606 | 3 | PTK2B | 2185 | 8 | 27242840 | ‘A/G’ | −0.48 | 6.65E−04 |
| RS1347744 | 3 | | | 4 | 166502051 | ‘A/G’ | −0.47 | 6.66E−04 |
| RS2167644 | 3 | LRRN6C | 158038 | 9 | 28076344 | ‘A/G’ | −0.54 | 6.68E−04 |
| RS12615237 | 3 | | | 2 | 44131231 | ‘C/T’ | −0.63 | 6.70E−04 |
| RS3750010 | 3 | FLJ10324 | 55698 | 7 | 4867717 | ‘A/G’ | 0.92 | 6.78E−04 |
| RS249740 | 3 | | | 5 | 141874445 | ‘C/T’ | −0.61 | 6.79E−04 |
| RS6870276 | 3 | | | 5 | 85590471 | ‘A/G’ | −0.48 | 6.83E−04 |
| RS4657284 | 3 | | | 1 | 161707341 | ‘A/G’ | −0.70 | 6.86E−04 |
| RS2306245 | 3 | LOC653983 | 653983 | 4 | 852156 | ‘A/G’ | 0.49 | 6.95E−04 |
| RS2072824 | 3 | JARID2 | 3720 | 6 | 15616089 | ‘C/T’ | 0.67 | 6.97E−04 |
| RS4465845 | 3 | | | 20 | 4402065 | ‘A/G’ | −0.64 | 7.02E−04 |
| RS10937103 | 3 | ATP11B | 23200 | 3 | 184096377 | ‘A/G’ | −0.52 | 7.02E−04 |
| RS4902242 | 3 | | | 14 | 63299842 | ‘C/T’ | −0.71 | 7.06E−04 |
| RS277037 | 3 | | | 8 | 132689881 | ‘C/T’ | 0.47 | 7.10E−04 |
| RS744651 | 3 | | | 17 | 56918030 | ‘C/T’ | −0.51 | 7.13E−04 |
| RS1324997 | 3 | | | 13 | 52282657 | ‘A/G’ | 0.68 | 7.14E−04 |
| RS9506903 | 3 | | | 13 | 22150146 | ‘C/T’ | −0.51 | 7.18E−04 |
| RS688630 | 3 | TCTEX1D1 | 200132 | 1 | 66995554 | ‘A/G’ | 0.46 | 7.22E−04 |
| RS1158717 | 3 | | | 6 | 115901074 | ‘C/T’ | −0.62 | 7.24E−04 |
| RS6965360 | 3 | | | 7 | 88818990 | ‘A/G’ | −0.98 | 7.26E−04 |
| RS2717229 | 3 | PDIA5 | 10954 | 3 | 124362066 | ‘C/T’ | 0.46 | 7.32E−04 |
| RS4651073 | 3 | XPR1 | 9213 | 1 | 178867222 | ‘A/G’ | −0.48 | 7.42E−04 |
| RS364612 | 3 | | | 8 | 94124310 | ‘C/T’ | −0.63 | 7.43E−04 |
| RS3774051 | 3 | EHHADH | 1962 | 3 | 186424419 | ‘A/G’ | 0.58 | 7.44E−04 |
| RS10223320 | 3 | | | 5 | 158506964 | ‘C/T’ | 0.46 | 7.46E−04 |
| RS10521767 | 3 | | | X | 130284232 | ‘C/T’ | 0.59 | 7.61E−04 |
| RS4777700 | 3 | LOC283682 | 283682 | 15 | 92118937 | ‘A/G’ | −0.65 | 7.63E−04 |
| RS4731214 | 3 | | | 7 | 124230141 | ‘A/G’ | −0.47 | 7.75E−04 |
| RS11132149 | 3 | ODZ3 | 55714 | 4 | 183907038 | ‘A/G’ | −0.47 | 7.77E−04 |
| RS6677410 | 3 | BLZF1 | 8548 | 1 | 167619919 | ‘C/T’ | −0.46 | 7.86E−04 |
| RS4283967 | 3 | | | 7 | 124327771 | ‘C/T’ | 0.47 | 7.92E−04 |
| RS987848 | 3 | | | 13 | 38779740 | ‘C/T’ | 0.51 | 7.96E−04 |
| RS3816599 | 3 | TSPAN13 | 27075 | 7 | 16782067 | ‘A/G’ | −0.51 | 7.97E−04 |
| RS903027 | 3 | MGC34646 | 157807 | 8 | 62571982 | ‘G/T’ | −0.66 | 8.04E−04 |
| RS859170 | 3 | | | 21 | 17549488 | ‘C/T’ | 0.50 | 8.09E−04 |
| RS373747 | 3 | | | 22 | 18535192 | ‘C/T’ | −0.46 | 8.10E−04 |
| RS2377098 | 3 | ESRRG | 2104 | 1 | 214782137 | ‘G/T’ | 0.48 | 8.13E−04 |
| RS246107 | 3 | P4HA2 | 8974 | 5 | 131574567 | ‘A/G’ | −0.49 | 8.14E−04 |
| RS189816 | 3 | ARHGAP15 | 55843 | 2 | 143607496 | ‘C/T’ | −0.49 | 8.19E−04 |
| RS7201164 | 3 | | | 16 | 61224753 | ‘A/G’ | −0.57 | 8.23E−04 |
| RS521331 | 3 | | | 10 | 8264856 | ‘C/T’ | 0.53 | 8.28E−04 |
| RS1335579 | 3 | TCEA2 | 6919 | 20 | 62161757 | ‘A/G’ | −0.49 | 8.32E−04 |
| RS11651563 | 3 | FOXK2 | 3607 | 17 | 78106672 | ‘C/T’ | 1.25 | 8.33E−04 |
| RS13080275 | 3 | CNTN4 | 152330 | 3 | 2721456 | ‘C/T’ | 0.50 | 8.34E−04 |
| RS6961292 | 3 | POT1 | 25913 | 7 | 124290425 | ‘A/G’ | 0.47 | 8.36E−04 |
| RS10515283 | 1 | | | 5 | 98121571 | ‘C/T’ | −0.53 | 8.38E−04 |
| RS1282129 | 3 | | | 1 | 111424262 | ‘A/G’ | 0.48 | 8.38E−04 |
| RS660048 | 3 | | | 3 | 76142189 | ‘C/T’ | 0.71 | 8.42E−04 |
| RS1812315 | 3 | | | 15 | 25522899 | ‘C/T’ | 0.61 | 8.50E−04 |
| RS6778227 | 3 | | | 3 | 182516536 | ‘C/T’ | 0.45 | 8.55E−04 |
| RS10264288 | 3 | LOC401398 | 401398 | 7 | 124361763 | ‘A/G’ | 0.47 | 8.61E−04 |
| RS11761669 | 3 | LOC401398 | 401398 | 7 | 124373537 | ‘A/G’ | 0.47 | 8.61E−04 |
| RS12112909 | 3 | LOC401398 | 401398 | 7 | 124372070 | ‘C/T’ | −0.47 | 8.61E−04 |
| RS11618001 | 3 | | | 13 | 66975757 | ‘A/G’ | −0.77 | 8.63E−04 |
| RS12538333 | 3 | POT1 | 25913 | 7 | 124295593 | ‘C/T’ | 0.47 | 8.64E−04 |
| RS6912194 | 3 | | | 6 | 115923850 | ‘A/G’ | −0.61 | 8.65E−04 |
| RS3848521 | 3 | | | 19 | 62264525 | ‘A/G’ | 0.53 | 8.65E−04 |
| RS2897074 | 3 | | | 4 | 155157135 | ‘A/C’ | −0.45 | 8.75E−04 |
| RS25890 | 3 | | | 5 | 131465461 | ‘A/G’ | −0.49 | 8.75E−04 |
| RS873833 | 3 | CABIN1 | 23523 | 22 | 22757878 | ‘C/T’ | 0.53 | 8.82E−04 |
| RS2267064 | 3 | LOC648941 | 648941 | 22 | 22874632 | ‘G/T’ | 0.53 | 8.82E−04 |
| RS462769 | 3 | MGC26885 | 124044 | 16 | 88290764 | ‘A/G’ | −0.46 | 8.86E−04 |
| RS774508 | 3 | | | 2 | 155155961 | ‘C/T’ | 0.83 | 8.93E−04 |
| RS1010491 | 3 | SP140 | 11262 | 2 | 230868765 | ‘A/G’ | 1.04 | 8.94E−04 |
| RS12666427 | 3 | POT1 | 25913 | 7 | 124266588 | ‘A/G’ | −0.47 | 8.98E−04 |
| RS4377885 | 3 | POT1 | 25913 | 7 | 124320916 | ‘C/T’ | −0.47 | 8.98E−04 |
| RS10228682 | 3 | POT1 | 25913 | 7 | 124325272 | ‘C/T’ | −0.47 | 8.98E−04 |
| RS1904975 | 3 | | | 7 | 124340038 | ‘A/G’ | −0.47 | 8.98E−04 |
| RS6973812 | 3 | POT1 | 25913 | 7 | 124293986 | ‘C/T’ | 0.47 | 8.98E−04 |
| RS13029963 | 3 | | | 2 | 122601190 | ‘A/G’ | 0.57 | 9.09E−04 |
| RS589281 | 3 | CACNA2D3 | 55799 | 3 | 54722284 | ‘C/T’ | 0.45 | 9.11E−04 |
| RS1673130 | 3 | | | 19 | 9996687 | ‘C/T’ | −0.47 | 9.15E−04 |
| RS10953026 | 3 | PFTK1 | 5218 | 7 | 90388127 | ‘A/G’ | 0.50 | 9.22E−04 |
| RS2717272 | 3 | | | 3 | 183954066 | ‘C/T’ | 0.45 | 9.22E−04 |
| RS1550357 | 3 | | | 5 | 124505300 | ‘C/T’ | 0.45 | 9.29E−04 |
| RS9558678 | 3 | | | 13 | 105554332 | ‘C/T’ | −0.66 | 9.37E−04 |
| RS9977890 | 3 | DSCAM | 1826 | 21 | 40921882 | ‘C/T’ | −0.51 | 9.37E−04 |
| RS6604632 | 3 | ESRRG | 2104 | 1 | 214774201 | ‘A/G’ | −0.45 | 9.43E−04 |
| RS2305913 | 3 | FBF1 | 85302 | 17 | 71434536 | ‘A/G’ | −0.48 | 9.45E−04 |
| RS3893376 | 3 | | | 4 | 15351172 | ‘C/T’ | −0.51 | 9.46E−04 |
| RS2834939 | 3 | | | 21 | 35754778 | ‘A/G’ | 0.50 | 9.47E−04 |
| RS2278089 | 3 | NMI | 9111 | 2 | 151854918 | ‘A/C’ | −0.47 | 9.59E−04 |
| RS7959334 | 3 | TMTC1 | 83857 | 12 | 29764061 | ‘C/T’ | 0.44 | 9.61E−04 |
| RS896169 | 3 | | | 7 | 131500210 | ‘A/C’ | −0.50 | 9.64E−04 |
| RS10116548 | 3 | | | 9 | 12224642 | ‘C/T’ | −0.46 | 9.68E−04 |
| RS12128593 | 3 | | | 1 | 66747467 | ‘C/T’ | −0.68 | 9.75E−04 |
| RS499899 | 3 | | | 6 | 20109726 | ‘C/T’ | −0.51 | 9.80E−04 |
| RS11695594 | 3 | ERBB4 | 2066 | 2 | 212035677 | ‘C/T’ | 0.50 | 9.80E−04 |
| RS6961441 | 3 | LOC401398 | 401398 | 7 | 124378107 | ‘A/G’ | −0.46 | 9.88E−04 |
| RS1871770 | 3 | LOC401398 | 401398 | 7 | 124418075 | ‘G/T’ | −0.46 | 9.88E−04 |
| RS7787605 | 3 | LOC401398 | 401398 | 7 | 124447218 | ‘A/G’ | 0.46 | 9.88E−04 |
| RS1893833 | 3 | | | 18 | 73221461 | ‘C/T’ | −0.51 | 9.97E−04 |
| RS7625913 | 3 | | | 3 | 76167310 | ‘A/C’ | 0.67 | 9.98E−04 |

dbSNP_rs_ID: SNP identification number in NCBI dbSNP database
Gene_locus: Gene locus and gene id as reported by NCBI dbSNP database build 126
Priority_date: SNP listed in 1: US 11/245,248 2: US 60/819,014 3: US 60/867,454
Sequence_ID: Sequence identification number
Position: Basepair Position, SNP physical position according to NCBI Human Genome Build 36.1
Variants: Alternate SNP alleles or their complementary nucleotides in the position indicated by dbSNP RS ID and basepair position
Minor Allele: SNP allele or its complementary nucleotide that is less common in the control population.
Allele_X2: Chi-squared test based on allele frequencies
Coefficient: Coefficient w of the model glm(z~w + r, family = binomial(link = logit)) in R where z is hypertension status, w is genotype (0, 1, 2) and r is T2D status
P value: P value of the coefficient w
Gene_content: Genes positioned within 100 Kbp up and downstream from the physical position of the SNPs based on NCBI Human Genome Build 36.1

TABLE 7
SNP markers with the strongest association with hypertension
in the regression analysis with a recessive genotype model and T2D
as a covariate. The analysis is based on the combined data of 110
HT cases and 110 healthy controls from the Ashkenazi Jew population,
114 HT cases and 114 healthy controls from the East Finnish population,
41 HT cases and 41 healthy controls from the German population and
28 HT cases and 28 healthy controls from the English population.

| | | Gene locus | | | | | | |
|---|---|---|---|---|---|---|---|---|
| | | and | | | | | | P |
| dbSNP rs ID | Priority date | Gene ID | | Chromosome | Position | Variats | Coefficient | value |
| RS10509557 | 3 | STAMBPL1 | 57559 | 10 | 90653819 | ‘C/T’ | −1.70 | 1.34E−06 |
| RS11088668 | 3 | | | 21 | 18448920 | ‘C/T’ | −1.00 | 1.15E−05 |
| RS1568447 | 3 | | | 17 | 70348607 | ‘A/G’ | −1.11 | 1.41E−05 |
| RS2458291 | 3 | ATP6V1C1 | 528 | 8 | 104138327 | ‘C/T’ | −0.93 | 1.92E−05 |
| RS8043993 | 3 | TMC7 | 79905 | 16 | 18956879 | ‘A/G’ | −0.85 | 1.96E−05 |
| RS1425531 | 3 | | | 4 | 143645481 | ‘C/T’ | 0.85 | 2.10E−05 |
| RS261988 | 3 | | | 5 | 95866641 | ‘A/G’ | 1.11 | 2.35E−05 |
| RS6474131 | 3 | LOC651362 | 651362 | 8 | 39344125 | ‘A/C’ | 0.99 | 2.53E−05 |
| RS6934805 | 3 | | | 6 | 168581640 | ‘A/G’ | 0.84 | 3.79E−05 |
| RS10088400 | 3 | ADAM5 | 255926 | 8 | 39350791 | ‘A/C’ | 0.96 | 4.07E−05 |
| RS2723167 | 3 | | | 2 | 113337681 | ‘C/T’ | −0.90 | 4.37E−05 |
| RS4481638 | 3 | LOC286094 | 286094 | 8 | 136363511 | ‘A/G’ | −1.22 | 5.48E−05 |
| RS717576 | 3 | STAMBPL1 | 57559 | 10 | 90657573 | ‘C/T’ | −1.30 | 5.69E−05 |
| RS7213057 | 3 | FLJ22222 | 79701 | 17 | 77972228 | ‘C/T’ | 1.78 | 6.70E−05 |
| RS12513906 | 3 | | | 5 | 80267341 | ‘G/T’ | −3.07 | 7.24E−05 |
| RS6435367 | 3 | | | 2 | 207804849 | ‘A/G’ | −1.11 | 7.94E−05 |
| RS1163665 | 3 | LIN7A | 8825 | 12 | 79813991 | ‘C/T’ | 0.86 | 8.03E−05 |
| RS11650418 | 3 | ABR | 29 | 17 | 1025021 | ‘G/T’ | 0.79 | 8.18E−05 |
| RS7539199 | 3 | | | 1 | 34855853 | ‘A/G’ | 0.92 | 8.19E−05 |
| RS1953352 | 3 | | | 14 | 55266912 | ‘A/G’ | 1.03 | 8.48E−05 |
| RS11690643 | 3 | | | 2 | 228500725 | ‘C/T’ | −0.78 | 8.65E−05 |
| RS1425533 | 3 | | | 4 | 143640625 | ‘C/T’ | 0.79 | 9.37E−05 |
| RS6128804 | 3 | | | 20 | 58403830 | ‘A/G’ | 0.81 | 1.08E−04 |
| RS10962917 | 3 | | | 9 | 17220086 | ‘A/G’ | 0.82 | 1.12E−04 |
| RS1335579 | 3 | TCEA2 | 6919 | 20 | 62161757 | ‘A/G’ | −0.78 | 1.24E−04 |
| RS4447608 | 3 | | | 2 | 113322428 | ‘C/T’ | −0.83 | 1.35E−04 |
| RS827228 | 3 | ARHGAP15 | 55843 | 2 | 143626189 | ‘C/T’ | 0.76 | 1.41E−04 |
| RS599367 | 3 | | | 1 | 20306989 | ‘C/T’ | −1.31 | 1.47E−04 |
| RS3127084 | 3 | NRAP | 4892 | 10 | 115366641 | ‘A/G’ | −0.94 | 1.51E−04 |
| RS7252391 | 3 | IGSF4C | 199731 | 19 | 48834611 | ‘A/G’ | 1.70 | 1.52E−04 |
| RS9533785 | 3 | | | 13 | 43661038 | ‘A/G’ | −0.80 | 1.56E−04 |
| RS17699211 | 3 | | | 12 | 3903752 | ‘C/T’ | 0.91 | 1.62E−04 |
| RS10954695 | 3 | PCLO | 27445 | 7 | 82395164 | ‘A/G’ | 0.75 | 1.62E−04 |
| RS12960602 | 3 | | | 18 | 501960 | ‘C/T’ | 0.75 | 1.69E−04 |
| RS1524909 | 3 | | | 2 | 156090205 | ‘A/G’ | −1.29 | 1.72E−04 |
| RS521331 | 3 | | | 10 | 8264856 | ‘C/T’ | 0.74 | 1.79E−04 |
| RS883509 | 3 | TSPAN5 | 10098 | 4 | 99782699 | ‘C/T’ | 1.06 | 1.87E−04 |
| RS1009283 | 3 | | | 2 | 240842754 | ‘A/G’ | 0.73 | 1.87E−04 |
| RS976714 | 3 | PCLO | 27445 | 7 | 82419795 | ‘C/T’ | 0.74 | 1.88E−04 |
| RS9863894 | 3 | NLGN1 | 22871 | 3 | 174960398 | ‘C/T’ | 0.73 | 1.90E−04 |
| RS6595959 | 3 | LOC649922 | 649922 | 5 | 97938006 | ‘C/T’ | 0.82 | 1.90E−04 |
| RS6474169 | 3 | ADAM18 | 8749 | 8 | 39697120 | ‘G/T’ | 0.78 | 2.02E−04 |
| RS7147000 | 3 | | | 14 | 104078860 | ‘A/G’ | −1.24 | 2.02E−04 |
| RS990060 | 3 | | | 6 | 89131055 | ‘C/T’ | 0.96 | 2.03E−04 |
| RS9344790 | 3 | | | 6 | 89116996 | ‘A/G’ | 0.96 | 2.03E−04 |
| RS10954696 | 3 | PCLO | 27445 | 7 | 82420782 | ‘C/T’ | 0.74 | 2.05E−04 |
| RS2523647 | 3 | | | 6 | 31557757 | ‘C/T’ | −1.62 | 2.05E−04 |
| RS2256182 | 3 | | | 8 | 93637241 | ‘C/T’ | 0.72 | 2.09E−04 |
| RS7081359 | 3 | MGMT | 4255 | 10 | 131402923 | ‘C/T’ | −0.72 | 2.11E−04 |
| RS873833 | 3 | CABIN1 | 23523 | 22 | 22757878 | ‘C/T’ | 1.64 | 2.15E−04 |
| RS2267064 | 3 | LOC648941 | 648941 | 22 | 22874632 | ‘G/T’ | 1.64 | 2.15E−04 |
| RS7334289 | 3 | | | 13 | 37000990 | ‘C/T’ | 0.81 | 2.19E−04 |
| RS6823763 | 3 | | | 4 | 77945322 | ‘A/G’ | 2.01 | 2.19E−04 |
| RS204505 | 3 | | | 9 | 117927770 | ‘C/T’ | −0.73 | 2.19E−04 |
| RS6074018 | 3 | MANBAL | 63905 | 20 | 35358710 | ‘A/G’ | −0.73 | 2.27E−04 |
| RS3863537 | 3 | | | X | 13030051 | ‘C/T’ | −0.94 | 2.28E−04 |
| RS7059239 | 3 | PPEF1 | 5475 | X | 18622368 | ‘A/G’ | −0.75 | 2.34E−04 |
| RS5909201 | 3 | PPEF1 | 5475 | X | 18623637 | ‘C/T’ | −0.75 | 2.34E−04 |
| RS2260849 | 3 | ABR | 29 | 17 | 943785 | ‘C/T’ | 0.77 | 2.38E−04 |
| RS1871164 | 3 | | | 5 | 152771658 | ‘C/T’ | −0.84 | 2.41E−04 |
| RS1158717 | 3 | | | 6 | 115901074 | ‘C/T’ | −0.77 | 2.42E−04 |
| RS2025245 | 3 | | | 13 | 37001577 | ‘A/G’ | 0.80 | 2.50E−04 |
| RS10116548 | 3 | | | 9 | 12224642 | ‘C/T’ | −0.76 | 2.66E−04 |
| RS4471434 | 3 | KIRREL3 | 84623 | 11 | 125892601 | ‘C/T’ | 0.97 | 2.73E−04 |
| RS10978931 | 3 | | | 9 | 109386549 | ‘A/G’ | −0.90 | 2.74E−04 |
| RS1017035 | 3 | PSCD3 | 9265 | 7 | 6174894 | ‘A/G’ | −1.33 | 2.78E−04 |
| RS12581363 | 3 | | | 12 | 86455427 | ‘C/T’ | 0.72 | 2.80E−04 |
| RS2377098 | 3 | ESRRG | 2104 | 1 | 214782137 | ‘G/T’ | 0.76 | 2.91E−04 |
| RS276855 | 3 | | | 15 | 37318605 | ‘A/G’ | −0.89 | 2.93E−04 |
| RS798646 | 3 | | | 7 | 23586259 | ‘C/T’ | −0.75 | 2.96E−04 |
| RS758439 | 3 | AFF2 | 2334 | X | 147872587 | ‘A/G’ | −0.94 | 2.98E−04 |
| RS6912194 | 3 | | | 6 | 115923850 | ‘A/G’ | −0.76 | 3.01E−04 |
| RS12599856 | 3 | TMC7 | 79905 | 16 | 18929949 | ‘A/G’ | −0.71 | 3.02E−04 |
| RS7463107 | 3 | ST18 | 9705 | 8 | 53350840 | ‘C/T’ | −1.42 | 3.08E−04 |
| RS2306245 | 3 | LOC653983 | 653983 | 4 | 852156 | ‘A/G’ | 0.93 | 3.18E−04 |
| RS6870276 | 3 | | | 5 | 85590471 | ‘A/G’ | −0.83 | 3.19E−04 |
| RS7805656 | 3 | LOC392670 | 392670 | 7 | 50148762 | ‘C/T’ | −0.89 | 3.34E−04 |
| RS17523117 | 3 | | | 5 | 124691426 | ‘A/G’ | −0.71 | 3.34E−04 |
| RS2359682 | 3 | LOC391475 | 391475 | 2 | 207288554 | ‘A/G’ | 0.71 | 3.40E−04 |
| RS6467917 | 3 | PCLO | 27445 | 7 | 82407593 | ‘A/G’ | 0.79 | 3.50E−04 |
| RS527713 | 3 | | | 6 | 94863650 | ‘C/T’ | −1.03 | 3.51E−04 |
| RS4432885 | 3 | LOC649120 | 649120 | 5 | 84156628 | ‘C/T’ | −1.09 | 3.55E−04 |
| RS857160 | 3 | C1orf168 | 199920 | 1 | 57014455 | ‘A/G’ | −1.36 | 3.58E−04 |
| RS2158232 | 3 | | | 16 | 17862049 | ‘G/T’ | −0.88 | 3.59E−04 |
| RS3130559 | 3 | PSORS1C1 | 170679 | 6 | 31205280 | ‘C/T’ | −0.71 | 3.62E−04 |
| RS1833036 | 3 | ESRRG | 2104 | 1 | 214773635 | ‘A/C’ | 0.75 | 3.63E−04 |
| RS4844078 | 3 | AFF2 | 2334 | X | 147873742 | ‘A/G’ | −0.93 | 3.63E−04 |
| RS10245474 | 3 | ATXN7L4 | 222255 | 7 | 105249944 | ‘A/C’ | 0.71 | 3.64E−04 |
| RS456509 | 3 | | | 5 | 98022934 | ‘A/G’ | 0.77 | 3.71E−04 |
| RS2409472 | 3 | | | 21 | 33421296 | ‘A/G’ | 0.98 | 3.74E−04 |
| RS11618001 | 3 | | | 13 | 66975757 | ‘A/G’ | −0.89 | 3.84E−04 |
| RS288193 | 3 | FBXL17 | 64839 | 5 | 107346510 | ‘A/G’ | −0.71 | 3.92E−04 |
| RS7557557 | 3 | BARD1 | 580 | 2 | 215342872 | ‘C/T’ | 0.84 | 3.93E−04 |
| RS4807030 | 3 | | | 19 | 5340941 | ‘A/G’ | 0.69 | 3.93E−04 |
| RS2062960 | 3 | LOC647489 | 647489 | 18 | 67533577 | ‘A/G’ | −0.77 | 3.95E−04 |
| RS2011050 | 3 | | | 5 | 151077474 | ‘A/G’ | 1.12 | 3.95E−04 |
| RS351211 | 3 | | | 15 | 72363918 | ‘G/T’ | −2.11 | 3.99E−04 |
| RS2341919 | 3 | AFF2 | 2334 | X | 147870833 | ‘G/T’ | −0.92 | 3.99E−04 |
| RS2055598 | 3 | CNTN5 | 53942 | 11 | 98794981 | ‘A/G’ | 1.32 | 4.00E−04 |
| RS6561018 | 3 | MGC40178 | 122046 | 13 | 30433891 | ‘A/G’ | −0.73 | 4.02E−04 |
| RS4245178 | 3 | TMPRSS13 | 84000 | 11 | 117293523 | ‘C/T’ | 1.12 | 4.12E−04 |
| RS3816272 | 3 | IMP-1 | 10642 | 17 | 44475466 | ‘C/T’ | −1.10 | 4.28E−04 |
| RS10831742 | 3 | MICAL2 | 9645 | 11 | 12126901 | ‘A/G’ | 2.34 | 4.30E−04 |
| RS9937539 | 3 | TMC7 | 79905 | 16 | 18941493 | ‘A/C’ | −0.70 | 4.35E−04 |
| RS6677410 | 3 | BLZF1 | 8548 | 1 | 167619919 | ‘C/T’ | −0.71 | 4.44E−04 |
| RS10464988 | 3 | LOC286094 | 286094 | 8 | 136363842 | ‘C/T’ | −0.94 | 4.51E−04 |
| RS942233 | 3 | | | 13 | 79599849 | ‘A/G’ | 0.84 | 4.55E−04 |
| RS9309828 | 3 | | | 3 | 79843464 | ‘A/G’ | 0.68 | 4.59E−04 |
| RS8063120 | 3 | | | 16 | 80352553 | ‘A/G’ | −0.70 | 4.59E−04 |
| RS725613 | 3 | KIAA0350 | 23274 | 16 | 11077184 | ‘A/C’ | −0.94 | 4.64E−04 |
| RS3816599 | 3 | TSPAN13 | 27075 | 7 | 16782067 | ‘A/G’ | −0.77 | 4.69E−04 |
| RS4260345 | 3 | THRB | 7068 | 3 | 24231702 | ‘C/T’ | 0.88 | 4.71E−04 |
| RS744651 | 3 | | | 17 | 56918030 | ‘C/T’ | −0.69 | 4.72E−04 |
| RS6547369 | 3 | | | 2 | 81477485 | ‘A/C’ | −1.01 | 4.75E−04 |
| RS578130 | 3 | ARRB1 | 408 | 11 | 74681211 | ‘C/T’ | −0.68 | 4.80E−04 |
| RS2173086 | 3 | KIAA1040 | 23041 | 12 | 61165237 | ‘A/G’ | 0.74 | 4.86E−04 |
| RS896169 | 3 | | | 7 | 131500210 | ‘A/C’ | −1.20 | 4.89E−04 |
| RS1447549 | 3 | | | 2 | 133334690 | ‘C/T’ | 1.58 | 4.89E−04 |
| RS39617 | 3 | TRIO | 7204 | 5 | 14263897 | ‘G/T’ | 0.68 | 4.95E−04 |
| RS6579891 | 3 | | | 5 | 151054802 | ‘C/T’ | 1.54 | 5.01E−04 |
| RS6461076 | 3 | DGKB | 1607 | 7 | 14225771 | ‘G/T’ | 0.96 | 5.01E−04 |
| RS890027 | 3 | | | 8 | 5844762 | ‘A/G’ | −1.47 | 5.06E−04 |
| RS1582029 | 3 | | | 9 | 17233746 | ‘A/G’ | 0.77 | 5.10E−04 |
| RS6504593 | 3 | IMP-1 | 10642 | 17 | 44487818 | ‘C/T’ | −0.79 | 5.17E−04 |
| RS1010032 | 3 | | | 3 | 59531242 | ‘C/T’ | 1.15 | 5.19E−04 |
| RS6806589 | 3 | | | 3 | 76231050 | ‘C/T’ | 1.04 | 5.19E−04 |
| RS1110968 | 3 | | | 4 | 165711289 | ‘A/C’ | −0.94 | 5.21E−04 |
| RS1895391 | 3 | SV2C | 22987 | 5 | 75481681 | ‘A/G’ | −0.73 | 5.32E−04 |
| RS1500106 | 3 | | | 12 | 124315518 | ‘C/T’ | 0.71 | 5.33E−04 |
| RS1488547 | 3 | NLGN1 | 22871 | 3 | 175008462 | ‘C/T’ | 0.67 | 5.35E−04 |
| RS3091629 | 3 | PKIG | 11142 | 20 | 42598785 | ‘G/T’ | 0.79 | 5.38E−04 |
| RS7909235 | 3 | CTNNA3 | 29119 | 10 | 68044090 | ‘G/T’ | −0.68 | 5.45E−04 |
| RS2243512 | 3 | | | 12 | 103343237 | ‘C/T’ | 0.71 | 5.45E−04 |
| RS725027 | 3 | ATP2B2 | 491 | 3 | 10402190 | ‘A/G’ | 0.71 | 5.47E−04 |
| RS10906855 | 3 | | | 10 | 15277222 | ‘A/G’ | 0.70 | 5.58E−04 |
| RS582447 | 3 | GRB14 | 2888 | 2 | 165074838 | ‘G/T’ | 0.70 | 5.59E−04 |
| RS6633148 | 3 | PPEF1 | 5475 | X | 18734999 | ‘C/T’ | −0.77 | 5.63E−04 |
| RS13029963 | 3 | | | 2 | 122601190 | ‘A/G’ | 0.71 | 5.71E−04 |
| RS6659761 | 3 | | | 1 | 173450562 | ‘C/T’ | −0.71 | 5.73E−04 |
| RS12495441 | 3 | | | 3 | 86459666 | ‘C/T’ | −0.70 | 5.76E−04 |
| RS51774234 | 3 | | | 10 | 30257521 | ‘C/T’ | 2.00 | 6.11E−04 |
| RS1998957 | 3 | TPP2 | 7174 | 13 | 102064784 | ‘A/G’ | −0.69 | 6.18E−04 |
| RS4955834 | 3 | | | 3 | 55198023 | ‘C/T’ | −0.72 | 6.27E−04 |
| RS7359067 | 3 | | | 14 | 55464946 | ‘C/T’ | −1.54 | 6.35E−04 |
| RS2901483 | 3 | | | 2 | 62618776 | ‘A/G’ | 0.76 | 6.47E−04 |
| RS939543 | 3 | TMEM104 | 54868 | 17 | 70303779 | ‘A/G’ | −0.76 | 6.61E−04 |
| RS10872824 | 3 | | | 10 | 133356838 | ‘A/G’ | 0.71 | 6.62E−04 |
| RS984923 | 3 | | | 8 | 24690660 | ‘A/G’ | 1.00 | 6.68E−04 |
| RS2072954 | 3 | PLCB4 | 5332 | 20 | 9388840 | ‘C/T’ | 1.10 | 6.72E−04 |
| RS7318557 | 3 | LHFP | 10186 | 13 | 38818286 | ‘C/T’ | −0.67 | 6.75E−04 |
| RS1073768 | 3 | | | 20 | 35310424 | ‘A/G’ | 0.72 | 6.78E−04 |
| RS873634 | 3 | HCN2 | 610 | 19 | 539305 | ‘G/T’ | 1.22 | 6.79E−04 |
| RS6663840 | 3 | GlyBP | 9731 | 1 | 3733179 | ‘A/G’ | −0.67 | 6.88E−04 |
| RS4585212 | 3 | | | 3 | 175925548 | ‘A/G’ | 0.73 | 6.93E−04 |
| RS12141192 | 3 | KIF1B | 23095 | 1 | 10353719 | ‘C/T’ | 0.69 | 6.95E−04 |
| RS983789 | 3 | | | 1 | 157862306 | ‘G/T’ | −0.72 | 6.97E−04 |
| RS9391970 | 3 | | | 6 | 2765877 | ‘A/G’ | 0.66 | 7.06E−04 |
| RS2595042 | 3 | | | 8 | 29979947 | ‘A/G’ | −0.80 | 7.07E−04 |
| RS5917222 | 3 | | | X | 38509735 | ‘C/T’ | −0.66 | 7.11E−04 |
| RS4237333 | 3 | | | 10 | 73307232 | ‘C/T’ | 0.75 | 7.16E−04 |
| RS7646664 | 3 | ATP2B2 | 491 | 3 | 10404108 | ‘C/T’ | 0.68 | 7.25E−04 |
| RS4481619 | 3 | ST18 | 9705 | 8 | 53335359 | ‘C/T’ | −1.55 | 7.39E−04 |
| RS959678 | 3 | ZPBP | 11055 | 7 | 50031156 | ‘A/G’ | −0.68 | 7.40E−04 |
| RS2697144 | 3 | SLC6A1 | 6529 | 3 | 11026099 | ‘A/G’ | 1.41 | 7.43E−04 |
| RS1673130 | 3 | | | 19 | 9996687 | ‘C/T’ | −0.67 | 7.51E−04 |
| RS26999 | 3 | MCTP1 | 79772 | 5 | 94261262 | ‘C/T’ | −0.73 | 7.56E−04 |
| RS354286 | 3 | TRIO | 7204 | 5 | 14288406 | ‘A/G’ | 0.65 | 7.57E−04 |
| RS1366315 | 3 | | | 5 | 67292325 | ‘A/G’ | −0.67 | 7.57E−04 |
| RS5908660 | 3 | | | X | 142334080 | ‘A/G’ | −1.06 | 7.58E−04 |
| RS2925725 | 3 | LOC651419 | 651419 | 5 | 6313651 | ‘A/C’ | −0.71 | 7.62E−04 |
| RS2838808 | 3 | ADARB1 | 104 | 21 | 45445534 | ‘C/T’ | 1.17 | 7.65E−04 |
| RS5934075 | 3 | | | X | 13077796 | ‘A/G’ | −0.87 | 7.67E−04 |
| RS244120 | 3 | PKIG | 11142 | 20 | 42629119 | ‘A/G’ | 0.77 | 7.68E−04 |
| RS7169075 | 3 | LRRC28 | 123355 | 15 | 97694447 | ‘A/G’ | 1.54 | 7.77E−04 |
| RS9804335 | 3 | | | 10 | 130137163 | ‘A/G’ | −0.67 | 7.85E−04 |
| RS9292501 | 3 | ADAMTS12 | 81792 | 5 | 33639167 | ‘A/G’ | 0.79 | 7.94E−04 |
| RS6710189 | 3 | | | 2 | 112506263 | ‘A/G’ | 1.42 | 7.96E−04 |
| RS32549 | 3 | TRIO | 7204 | 5 | 14266135 | ‘A/G’ | 0.66 | 8.03E−04 |
| RS9695286 | 3 | | | 9 | 109812888 | ‘C/T’ | −0.73 | 8.12E−04 |
| RS6500316 | 3 | | | 16 | 49108242 | ‘A/G’ | −0.99 | 8.35E−04 |
| RS570657 | 3 | GRB14 | 2888 | 2 | 165064589 | ‘C/T’ | 0.68 | 8.35E−04 |
| RS7406978 | 3 | ABR | 29 | 17 | 983909 | ‘C/T’ | −0.71 | 8.41E−04 |
| RS1928863 | 3 | | | 9 | 12240170 | ‘A/G’ | −0.69 | 8.43E−04 |
| RS6073964 | 3 | | | 20 | 35342708 | ‘A/G’ | −0.66 | 8.43E−04 |
| RS532040 | 3 | | | 11 | 129081186 | ‘C/T’ | −0.67 | 8.43E−04 |
| RS6121666 | 3 | CDH4 | 1002 | 20 | 59535349 | ‘A/G’ | −0.80 | 8.45E−04 |
| RS2068259 | 3 | C20orf74 | 57186 | 20 | 20341219 | ‘A/C’ | −0.66 | 8.47E−04 |
| RS1838733 | 3 | PDE4D | 5144 | 5 | 58569149 | ‘A/G’ | −0.65 | 8.52E−04 |
| RS4107736 | 3 | | | 8 | 29995506 | ‘A/G’ | −0.84 | 8.59E−04 |
| RS5911500 | 3 | | | X | 115726187 | ‘C/T’ | −0.85 | 8.60E−04 |
| RS1022790 | 3 | | | 20 | 10678759 | ‘A/G’ | 0.79 | 8.61E−04 |
| RS11733672 | 3 | | | 4 | 4653806 | ‘C/T’ | −0.66 | 8.83E−04 |
| RS9342944 | 3 | RIMS1 | 22999 | 6 | 73131044 | ‘C/T’ | −0.65 | 8.85E−04 |
| RS901538 | 3 | | | 11 | 98312568 | ‘C/T’ | −0.88 | 8.86E−04 |
| RS462769 | 3 | MGC26885 | 124044 | 16 | 88290764 | ‘A/G’ | −0.68 | 8.95E−04 |
| RS4952002 | 3 | LYCAT | 253558 | 2 | 30713511 | ‘A/G’ | −0.68 | 8.97E−04 |
| RS10494494 | 3 | | | 1 | 174140762 | ‘C/T’ | −0.67 | 9.15E−04 |
| RS13149290 | 3 | LOC152485 | 152485 | 4 | 146970416 | ‘C/T’ | −0.66 | 9.17E−04 |
| RS4709122 | 3 | RPS6KA2 | 6196 | 6 | 166907733 | ‘C/T’ | −0.67 | 9.21E−04 |
| RS7393306 | 3 | C10orf92 | 54777 | 10 | 134483343 | ‘A/G’ | −0.74 | 9.26E−04 |
| RS7571570 | 3 | DTNB | 1838 | 2 | 25475034 | ‘C/T’ | 0.65 | 9.29E−04 |
| RS917684 | 3 | | | 4 | 10961129 | ‘A/G’ | 1.96 | 9.30E−04 |
| RS7560587 | 3 | KIAA1679 | 80731 | 2 | 138118624 | ‘C/T’ | −1.13 | 9.32E−04 |
| RS3027363 | 3 | GLRA2 | 2742 | X | 14519924 | ‘A/G’ | −0.85 | 9.37E−04 |
| RS4846217 | 3 | | | 1 | 10374386 | ‘C/T’ | 0.66 | 9.52E−04 |
| RS893911 | 3 | | | 15 | 64715542 | ‘C/T’ | −0.68 | 9.56E−04 |
| RS1880832 | 3 | | | 17 | 36137258 | ‘A/G’ | 0.78 | 9.74E−04 |
| RS1559621 | 3 | | | 2 | 59994112 | ‘A/G’ | 1.31 | 9.77E−04 |
| RS9819838 | 3 | LOC344595 | 344595 | 3 | 108418493 | ‘A/C’ | −1.10 | 9.79E−04 |
| RS6902101 | 3 | | | 6 | 140626077 | ‘A/G’ | −0.71 | 9.87E−04 |
| RS921449 | 3 | | | 8 | 35086721 | ‘C/T’ | −0.96 | 9.96E−04 |
| RS4478858 | 3 | | | 1 | 31656512 | ‘A/G’ | −0.77 | 9.97E−04 |

dbSNP_rs_ID: SNP identification number in NCBI dbSNP database
Gene_locus: Gene locus and gene id as reported by NCBI dbSNP database build 126
Priority_date: SNP listed in 1: US 11/245,248 2: US 60/819,014 3: US 60/867,454
Sequence_ID: Sequence identification number
Position: Basepair Position, SNP physical position according to NCBI Human Genome Build 36.1
Variants: Alternate SNP alleles or their complementary nucleotides in the position indicated by dbSNP RS ID and basepair position
Minor Allele: SNP allele or its complementary nucleotide that is less common in the control population.
Allele_X2: Chi-squared test based on allele frequencies
Coefficient: Coefficient w of the model g1m(z~w + r, family = binomial(link = logit)) in R where z is hypertension status, w is genotype (0, 1, 1<--2) and r is T2D status
P value: P value of the coefficient w
Gene_content: Genes positioned within 100 Kbp up and downstream from the physical position of the SNPs based on NCBI Human Genome Build 36.1

TABLE 8
SNP markers with the strongest association with hypertension
in the regression analysis with a dominant genotype model and T2D as
a covariate. The analysis is based on the combined data of 110 HT
cases and 110 healthy controls from the Ashkenazi Jew population,
114 HT cases and 114 healthy controls from the East Finnish population,
41 HT cases and 41 healthy controls from the German population
and 28 HT cases and 28 healthy controls from the English population.

| | | Gene locus | | | | | | |
|---|---|---|---|---|---|---|---|---|
| | | and | | | | | | |
| dbSNP rs ID | Priority date | Gene ID | | Chromosome | Position | Variats | Coefficient | P value |
| RS165774 | 3 | COMT | 1312 | 22 | 18332561 | ‘A/G’ | 1.80 | 2.45E−06 |
| RS6444191 | 3 | ST6GAL1 | 6480 | 3 | 188182304 | ‘A/G’ | −0.98 | 6.23E−06 |
| RS4813231 | 3 | | | 20 | 16551596 | ‘A/G’ | −1.13 | 1.61E−05 |
| RS10241873 | 3 | ZNF212 | 7988 | 7 | 148571806 | ‘A/C’ | 1.14 | 2.06E−05 |
| RS934083 | 3 | CACNA2D3 | 55799 | 3 | 54765419 | ‘C/T’ | 0.94 | 3.47E−05 |
| RS2242278 | 3 | SPON2 | 10417 | 4 | 1155516 | ‘A/G’ | −1.53 | 4.59E−05 |
| RS711129 | 3 | | | 12 | 76568088 | ‘C/T’ | −0.96 | 5.54E−05 |
| RS4891635 | 3 | | | 18 | 63741526 | ‘C/T’ | 1.10 | 6.50E−05 |
| RS2245192 | 3 | | | 7 | 113789771 | ‘C/T’ | −0.86 | 6.58E−05 |
| RS1795502 | 3 | LIN7A | 8825 | 12 | 79791804 | ‘A/C’ | −0.86 | 8.03E−05 |
| RS4760980 | 3 | | | 12 | 126628924 | ‘A/C’ | −0.80 | 8.25E−05 |
| RS4865755 | 3 | ITGA2 | 3673 | 5 | 52326944 | ‘C/T’ | −0.76 | 8.69E−05 |
| RS3900775 | 3 | SMOC2 | 64094 | 6 | 168613353 | ‘C/T’ | −0.84 | 9.57E−05 |
| RS6980380 | 3 | PRKAG2 | 51422 | 7 | 151018453 | ‘C/T’ | 0.83 | 9.85E−05 |
| RS6604634 | 3 | ESRRG | 2104 | 1 | 214787878 | ‘A/G’ | −0.91 | 1.24E−04 |
| RS7008482 | 3 | C8orf36 | 286053 | 8 | 126336812 | ‘G/T’ | 0.75 | 1.29E−04 |
| RS6038092 | 3 | | | 20 | 5137472 | ‘A/G’ | −0.96 | 1.38E−04 |
| RS7305776 | 3 | | | 12 | 76122239 | ‘A/G’ | 0.75 | 1.47E−04 |
| RS827226 | 3 | ARHGAP15 | 55843 | 2 | 143639083 | ‘C/T’ | −0.76 | 1.48E−04 |
| RS10119193 | 3 | | | 9 | 109336507 | ‘C/T’ | 0.87 | 1.53E−04 |
| RS4723924 | 3 | CDC2L5 | 8621 | 7 | 39997013 | ‘C/T’ | 0.76 | 1.57E−04 |
| RS720295 | 3 | PSAP | 5660 | 10 | 73267358 | ‘C/T’ | −0.78 | 1.62E−04 |
| RS290048 | 3 | | | 2 | 77381720 | ‘A/G’ | −1.45 | 1.69E−04 |
| RS6726521 | 3 | | | 2 | 236019083 | ‘A/G’ | −1.00 | 1.71E−04 |
| RS2329727 | 3 | | | 7 | 51368212 | ‘A/G’ | 1.05 | 1.76E−04 |
| RS3827256 | 3 | PFKL | 5211 | 21 | 44565251 | ‘A/G’ | −1.02 | 1.86E−04 |
| RS2216374 | 3 | | | 2 | 207798149 | ‘A/G’ | 1.07 | 1.91E−04 |
| RS10506851 | 3 | PPFIA2 | 8499 | 12 | 80662290 | ‘A/G’ | −0.79 | 2.05E−04 |
| RS7406978 | 3 | ABR | 29 | 17 | 983909 | ‘C/T’ | −0.86 | 2.11E−04 |
| RS11652097 | 3 | LOC649173 | 649173 | 17 | 42671716 | ‘C/T’ | −1.20 | 2.11E−04 |
| RS2675494 | 3 | EPHB2 | 2048 | 1 | 23099753 | ‘C/T’ | −0.89 | 2.17E−04 |
| RS7335400 | 3 | | | 13 | 37001152 | ‘A/C’ | −0.81 | 2.19E−04 |
| RS10495026 | 3 | ESRRG | 2104 | 1 | 214767553 | ‘C/T’ | −0.74 | 2.21E−04 |
| RS7141 | 3 | EFNB3 | 1949 | 17 | 7555326 | ‘A/G’ | 0.72 | 2.22E−04 |
| RS897407 | 3 | | | 8 | 136498130 | ‘A/G’ | 1.30 | 2.26E−04 |
| RS2182703 | 3 | | | 1 | 22899076 | ‘A/G’ | −0.71 | 2.27E−04 |
| RS11861084 | 3 | FANCA | 2175 | 16 | 88403211 | ‘A/C’ | 0.97 | 2.59E−04 |
| RS1369704 | 3 | DTNB | 1838 | 2 | 25477094 | ‘A/G’ | −0.71 | 2.61E−04 |
| RS11791609 | 3 | | | 9 | 12246453 | ‘A/G’ | 0.74 | 2.67E−04 |
| RS583190 | 3 | CACNA2D3 | 55799 | 3 | 54702117 | ‘A/C’ | 0.82 | 2.75E−04 |
| RS6816464 | 3 | | | 4 | 19537700 | ‘C/T’ | 0.71 | 2.76E−04 |
| RS10008492 | 3 | | | 4 | 38442115 | ‘C/T’ | −0.83 | 2.79E−04 |
| RS846491 | 3 | KATNAL1 | 84056 | 13 | 29743131 | ‘C/T’ | 0.89 | 2.99E−04 |
| RS6890771 | 3 | | | 5 | 180014372 | ‘C/T’ | 0.86 | 3.06E−04 |
| RS2852217 | 3 | GRIK4 | 2900 | 11 | 120152436 | ‘C/T’ | 0.70 | 3.08E−04 |
| RS149999 | 3 | | | 3 | 139027068 | ‘C/T’ | −1.03 | 3.10E−04 |
| RS9564765 | 3 | | | 13 | 70431786 | ‘A/G’ | 0.69 | 3.10E−04 |
| RS3780087 | 3 | AGPAT5 | 55326 | 8 | 6555609 | ‘C/T’ | 0.91 | 3.11E−04 |
| RS1880845 | 3 | | | 12 | 104377662 | ‘A/G’ | −1.38 | 3.14E−04 |
| RS1384634 | 3 | ZPBP | 11055 | 7 | 50035023 | ‘C/T’ | 0.86 | 3.14E−04 |
| RS1874622 | 3 | CRISPLD2 | 83716 | 16 | 83455234 | ‘A/G’ | −0.90 | 3.17E−04 |
| RS549065 | 3 | | | 6 | 94860908 | ‘G/T’ | 1.03 | 3.18E−04 |
| RS16956762 | 3 | | | 15 | 29539275 | ‘A/G’ | −1.17 | 3.21E−04 |
| RS9616080 | 3 | | | 22 | 45396830 | ‘C/T’ | −1.78 | 3.22E−04 |
| RS2137490 | 3 | | | 3 | 86393288 | ‘A/C’ | 0.74 | 3.39E−04 |
| RS1322280 | 3 | | | 9 | 10572246 | ‘C/T’ | −1.70 | 3.43E−04 |
| RS7590833 | 3 | HDAC4 | 9759 | 2 | 239948271 | ‘A/G’ | 0.70 | 3.45E−04 |
| RS6038010 | 3 | SLC23A2 | 9962 | 20 | 4811786 | ‘C/T’ | 0.73 | 3.45E−04 |
| RS7237611 | 3 | CD226 | 10666 | 18 | 65700586 | ‘A/G’ | −0.70 | 3.47E−04 |
| RS2072824 | 3 | JARID2 | 3720 | 6 | 15616089 | ‘C/T’ | 0.77 | 3.48E−04 |
| RS4900672 | 3 | | | 14 | 44967080 | ‘A/G’ | −0.93 | 3.53E−04 |
| RS7601055 | 3 | KIAA1486 | 57624 | 2 | 226028326 | ‘C/T’ | −1.02 | 3.55E−04 |
| RS2703833 | 3 | KCTD8 | 386617 | 4 | 44036287 | ‘C/T’ | 0.74 | 3.57E−04 |
| RS189816 | 3 | ARHGAP15 | 55843 | 2 | 143607496 | ‘C/T’ | −0.70 | 3.74E−04 |
| RS246107 | 3 | P4HA2 | 8974 | 5 | 131574567 | ‘A/G’ | −0.69 | 3.90E−04 |
| RS3848521 | 3 | | | 19 | 62264525 | ‘A/G’ | 0.69 | 3.94E−04 |
| RS4422314 | 3 | | | 3 | 166030858 | ‘A/G’ | −0.90 | 4.03E−04 |
| RS12128593 | 3 | | | 1 | 66747467 | ‘C/T’ | −0.82 | 4.03E−04 |
| RS320379 | 3 | | | 1 | 211743714 | ‘A/G’ | 0.70 | 4.14E−04 |
| RS1110277 | 3 | SLC23A2 | 9962 | 20 | 4802682 | ‘A/G’ | 0.69 | 4.28E−04 |
| RS5936438 | 3 | AFF2 | 2334 | X | 147694315 | ‘A/G’ | 0.92 | 4.29E−04 |
| RS4855268 | 3 | | | 3 | 166063217 | ‘C/T’ | −0.90 | 4.30E−04 |
| RS6436553 | 3 | KIAA1486 | 57624 | 2 | 226002129 | ‘A/G’ | −1.00 | 4.34E−04 |
| RS2637988 | 3 | IL1RN | 3557 | 2 | 113593250 | ‘A/G’ | −0.74 | 4.60E−04 |
| RS2051089 | 3 | | | 6 | 7569571 | ‘C/T’ | −0.68 | 4.61E−04 |
| RS921924 | 3 | | | 12 | 123787936 | ‘A/G’ | 0.78 | 4.62E−04 |
| RS12038863 | 3 | | | 1 | 178843066 | ‘A/C’ | −0.94 | 4.64E−04 |
| RS4663588 | 3 | | | 2 | 236042298 | ‘A/G’ | −0.99 | 4.67E−04 |
| RS767460 | 3 | CNTN4 | 152330 | 3 | 2716787 | ‘A/G’ | −0.68 | 4.74E−04 |
| RS9321194 | 3 | C6orf191 | 253582 | 6 | 130216831 | ‘C/T’ | −0.67 | 4.74E−04 |
| RS10120248 | 3 | | | 9 | 109897328 | ‘A/G’ | 1.53 | 4.75E−04 |
| RS1412435 | 3 | | | 9 | 109904257 | ‘C/T’ | 1.53 | 4.75E−04 |
| RS7991284 | 3 | | | 13 | 20584944 | ‘A/G’ | −0.82 | 4.78E−04 |
| RS2041670 | 3 | KIAA0350 | 23274 | 16 | 11082153 | ‘C/T’ | 1.02 | 4.79E−04 |
| RS1399333 | 3 | | | 4 | 10878977 | ‘C/T’ | 0.69 | 4.91E−04 |
| RS2305913 | 3 | FBF1 | 85302 | 17 | 71434536 | ‘A/G’ | −0.69 | 4.92E−04 |
| RS7624656 | 3 | PCOLCE2 | 26577 | 3 | 144068492 | ‘C/T’ | 0.94 | 4.96E−04 |
| RS4626202 | 3 | | | 4 | 19592202 | ‘C/T’ | 0.73 | 4.98E−04 |
| RS10515283 | 1 | | | 5 | 98121571 | ‘C/T’ | −0.69 | 5.06E−04 |
| RS1348530 | 3 | | | 4 | 162413018 | ‘C/T’ | 0.78 | 5.10E−04 |
| RS460879 | 3 | PCOLN3 | 5119 | 16 | 88240390 | ‘C/T’ | 0.78 | 5.10E−04 |
| RS11985201 | 3 | | | 8 | 39558078 | ‘A/G’ | −0.92 | 5.10E−04 |
| RS2283712 | 3 | PPEF1 | 5475 | X | 18752705 | ‘A/G’ | 0.87 | 5.12E−04 |
| RS9514497 | 3 | | | 13 | 105569397 | ‘A/G’ | −0.74 | 5.14E−04 |
| RS1449994 | 3 | | | 3 | 36136098 | ‘G/T’ | −0.68 | 5.18E−04 |
| RS1332855 | 3 | | | 9 | 82827607 | ‘G/T’ | −0.79 | 5.18E−04 |
| RS10521767 | 3 | | | X | 130284232 | ‘C/T’ | 0.88 | 5.20E−04 |
| RS477558 | 3 | | | 1 | 18092414 | ‘A/G’ | −0.73 | 5.23E−04 |
| RS5930664 | 3 | | | X | 125213018 | ‘A/G’ | 0.97 | 5.34E−04 |
| RS1387389 | 3 | PBX1 | 5087 | 1 | 162956386 | ‘C/T’ | −1.13 | 5.47E−04 |
| RS6075078 | 3 | | | 20 | 16519466 | ‘G/T’ | −0.77 | 5.52E−04 |
| RS1721355 | 3 | WDR69 | 164781 | 2 | 228491220 | ‘A/G’ | −0.70 | 5.55E−04 |
| RS3743024 | 3 | MAPKBP1 | 23005 | 15 | 39906263 | ‘C/T’ | −0.68 | 5.55E−04 |
| RS12487554 | 3 | SEC22L2 | 26984 | 3 | 124425472 | ‘A/G’ | 0.69 | 5.56E−04 |
| RS183007 | 3 | PPEF1 | 5475 | X | 18746756 | ‘C/T’ | 0.77 | 5.63E−04 |
| RS263350 | 3 | | | 5 | 95885359 | ‘C/T’ | −0.69 | 5.64E−04 |
| RS1641394 | 3 | LOC649004 | 649004 | 2 | 150315150 | ‘A/G’ | −1.19 | 5.69E−04 |
| RS2773857 | 3 | | | 9 | 12362356 | ‘A/G’ | 0.79 | 5.71E−04 |
| RS2655074 | 3 | | | 11 | 11157434 | ‘G/T’ | −1.05 | 5.72E−04 |
| RS11199496 | 3 | | | 10 | 122439906 | ‘C/T’ | −0.68 | 5.74E−04 |
| RS4143444 | 3 | | | 6 | 91103018 | ‘C/T’ | −0.84 | 5.76E−04 |
| RS11025056 | 3 | | | 11 | 19186741 | ‘A/G’ | −0.93 | 5.78E−04 |
| RS6984551 | 3 | LOC648814 | 648814 | 8 | 9148232 | ‘C/T’ | 0.68 | 5.80E−04 |
| RS2167644 | 3 | LRRN6C | 158038 | 9 | 28076344 | ‘A/G’ | −0.68 | 5.86E−04 |
| RS9639874 | 3 | HECW1 | 23072 | 7 | 43381531 | ‘A/G’ | −1.07 | 5.88E−04 |
| RS9558678 | 3 | | | 13 | 105554332 | ‘C/T’ | −0.76 | 5.89E−04 |
| RS7148858 | 3 | CLMN | 79789 | 14 | 94818468 | ‘C/T’ | −0.69 | 5.90E−04 |
| RS2191031 | 3 | | | 3 | 45885874 | ‘C/T’ | −0.97 | 5.95E−04 |
| RS261966 | 3 | | | 5 | 95875343 | ‘A/G’ | −0.75 | 5.96E−04 |
| RS7835480 | 3 | | | 8 | 131593803 | ‘A/G’ | −0.66 | 5.97E−04 |
| RS1544452 | 3 | | | 7 | 124168925 | ‘A/G’ | −0.68 | 5.98E−04 |
| RS2838817 | 3 | ADARB1 | 104 | 21 | 45456126 | ‘C/T’ | −1.26 | 6.01E−04 |
| RS12359135 | 3 | | | 10 | 94833525 | ‘C/T’ | −0.79 | 6.01E−04 |
| RS4873814 | 3 | | | 8 | 144793335 | ‘A/G’ | 0.76 | 6.03E−04 |
| RS7991184 | 3 | LOC642172 | 642172 | 13 | 86898647 | ‘A/C’ | −0.88 | 6.11E−04 |
| RS152439 | 3 | | | 5 | 141904579 | ‘C/T’ | 0.94 | 6.13E−04 |
| RS9518797 | 3 | TPP2 | 7174 | 13 | 102060880 | ‘C/T’ | 0.69 | 6.18E−04 |
| RS2303518 | 3 | MAPKBP1 | 23005 | 15 | 39897267 | ‘A/C’ | −0.67 | 6.20E−04 |
| RS6520049 | 3 | | | 22 | 45374973 | ‘C/T’ | −1.54 | 6.23E−04 |
| RS6604632 | 3 | ESRRG | 2104 | 1 | 214774201 | ‘A/G’ | −0.67 | 6.44E−04 |
| RS5979317 | 3 | MID1 | 4281 | X | 10462458 | ‘C/T’ | 0.83 | 6.47E−04 |
| RS1398882 | 3 | | | 17 | 39103977 | ‘C/T’ | −0.79 | 6.52E−04 |
| RS2327935 | 3 | HECA | 51696 | 6 | 139532786 | ‘A/G’ | −1.04 | 6.63E−04 |
| RS9945206 | 3 | | | 18 | 11222515 | ‘C/T’ | −0.95 | 6.65E−04 |
| RS9545836 | 3 | | | 13 | 81171954 | ‘A/G’ | −1.28 | 6.73E−04 |
| RS4399918 | 3 | NLGN1 | 22871 | 3 | 175087027 | ‘C/T’ | −0.86 | 6.76E−04 |
| RS10492519 | 3 | FLJ30707 | 220108 | 13 | 50722329 | ‘A/G’ | 0.68 | 6.77E−04 |
| RS25890 | 3 | | | 5 | 131465461 | ‘A/G’ | −0.67 | 6.87E−04 |
| RS10513560 | 3 | PPM1L | 151742 | 3 | 162193492 | ‘A/G’ | 0.96 | 7.04E−04 |
| RS6517708 | 3 | | | 21 | 16972413 | ‘A/G’ | 1.07 | 7.19E−04 |
| RS1406076 | 3 | HS3ST1 | 9957 | 4 | 11012802 | ‘C/T’ | −1.23 | 7.19E−04 |
| RS288649 | 3 | | | 16 | 61163939 | ‘C/T’ | 0.67 | 7.29E−04 |
| RS9922975 | 3 | | | 16 | 26847848 | ‘A/G’ | 0.78 | 7.38E−04 |
| RS13074723 | 3 | NLGN1 | 22871 | 3 | 175004791 | ‘A/G’ | −0.66 | 7.43E−04 |
| RS7565864 | 3 | | | 2 | 44135829 | ‘A/G’ | 0.81 | 7.45E−04 |
| RS1873773 | 3 | | | 4 | 54894535 | ‘A/G’ | −0.66 | 7.56E−04 |
| RS10738168 | 3 | | | 9 | 10570326 | ‘G/T’ | −1.36 | 7.61E−04 |
| RS11095604 | 3 | | | X | 13053820 | ‘C/T’ | 0.87 | 7.67E−04 |
| RS11794056 | 3 | SNX30 | 401548 | 9 | 114644464 | ‘A/G’ | −0.66 | 7.68E−04 |
| RS6843684 | 3 | | | 4 | 174859417 | ‘A/G’ | −0.71 | 7.71E−04 |
| RS4651073 | 3 | XPR1 | 9213 | 1 | 178867222 | ‘A/G’ | −0.66 | 7.72E−04 |
| RS4412655 | 3 | PLEKHA6 | 22874 | 1 | 202477373 | ‘A/C’ | 0.89 | 7.79E−04 |
| RS2391335 | 3 | EFNB2 | 1948 | 13 | 105969986 | ‘G/T’ | −0.82 | 7.87E−04 |
| RS1293427 | 3 | ZNF218 | 128553 | 20 | 51171008 | ‘A/G’ | 0.97 | 7.88E−04 |
| RS6885761 | 3 | | | 5 | 154595473 | ‘A/G’ | 0.99 | 7.90E−04 |
| RS592048 | 3 | TNS3 | 64759 | 7 | 47544477 | ‘C/T’ | 0.67 | 8.01E−04 |
| RS2246815 | 3 | DPYS | 1807 | 8 | 105509638 | ‘A/G’ | −1.17 | 8.06E−04 |
| RS987848 | 3 | | | 13 | 38779740 | ‘C/T’ | 0.67 | 8.11E−04 |
| RS6083269 | 3 | | | 20 | 23733807 | ‘A/G’ | −0.97 | 8.13E−04 |
| RS10494301 | 3 | KCNN3 | 3782 | 1 | 152967842 | ‘A/G’ | −0.96 | 8.19E−04 |
| RS4242670 | 3 | TEK | 7010 | 9 | 27190738 | ‘A/C’ | −1.69 | 8.24E−04 |
| RS11651563 | 3 | FOXK2 | 3607 | 17 | 78106672 | ‘C/T’ | 1.25 | 8.33E−04 |
| RS2097585 | 3 | | | 8 | 18063184 | ‘C/T’ | 0.84 | 8.37E−04 |
| RS1921931 | 3 | | | X | 13079476 | ‘A/G’ | 0.86 | 8.42E−04 |
| RS6041592 | 3 | | | 20 | 12673486 | ‘A/G’ | −0.66 | 8.44E−04 |
| RS11199460 | 3 | | | 10 | 122402920 | ‘A/G’ | −0.76 | 8.59E−04 |
| RS306364 | 3 | | | 4 | 131871491 | ‘A/G’ | −0.88 | 8.59E−04 |
| RS2183869 | 3 | APTX | 54840 | 9 | 32973442 | ‘A/G’ | −0.81 | 8.61E−04 |
| RS189947 | 3 | | | 21 | 17556641 | ‘A/C’ | 1.09 | 8.63E−04 |
| RS193762 | 3 | | | 16 | 11219674 | ‘A/C’ | 1.07 | 8.67E−04 |
| RS8084310 | 3 | | | 18 | 8940504 | ‘A/G’ | 0.72 | 8.70E−04 |
| RS894911 | 3 | CDH12 | 1010 | 5 | 22467224 | ‘C/T’ | −0.65 | 8.71E−04 |
| RS945864 | 3 | | | 9 | 1248905 | ‘C/T’ | −0.67 | 8.78E−04 |
| RS1476240 | 3 | ZPBP | 11055 | 7 | 50003183 | ‘A/G’ | 0.66 | 8.83E−04 |
| RS1531812 | 3 | | | X | 5712016 | ‘C/T’ | 1.07 | 8.86E−04 |
| RS10082730 | 3 | | | 12 | 44288969 | ‘C/T’ | 0.79 | 8.86E−04 |
| RS6534677 | 3 | | | 4 | 129441351 | ‘C/T’ | −0.84 | 8.96E−04 |
| RS10491285 | 3 | LOC648089 | 648089 | 5 | 126367985 | ‘A/G’ | 0.64 | 9.06E−04 |
| RS354694 | 3 | ARHGAP15 | 55843 | 2 | 143641987 | ‘C/T’ | −0.65 | 9.11E−04 |
| RS4657284 | 3 | | | 1 | 161707341 | ‘A/G’ | −0.74 | 9.18E−04 |
| RS4669621 | 3 | ATP6V1C2 | 245973 | 2 | 10803549 | ‘C/T’ | −0.70 | 9.20E−04 |
| RS7190823 | 3 | FANCA | 2175 | 16 | 88393544 | ‘C/T’ | 0.67 | 9.23E−04 |
| RS4476727 | 3 | | | 5 | 3340958 | ‘A/G’ | 1.17 | 9.24E−04 |
| RS1250126 | 3 | | | 4 | 1181042 | ‘C/T’ | −1.18 | 9.24E−04 |
| RS1822454 | 3 | PTPRM | 5797 | 18 | 7848157 | ‘A/G’ | 0.69 | 9.34E−04 |
| RS926073 | 3 | | | 21 | 35778797 | ‘A/G’ | 0.74 | 9.36E−04 |
| RS2447523 | 3 | | | 11 | 33418920 | ‘A/G’ | −0.68 | 9.42E−04 |
| RS7905355 | 3 | | | 10 | 125914585 | ‘A/G’ | −0.65 | 9.56E−04 |
| RS4680 | 3 | COMT | 1312 | 22 | 18331271 | ‘A/G’ | 0.74 | 9.67E−04 |
| RS9298628 | 3 | | | 8 | 42725148 | ‘C/T’ | 1.79 | 9.71E−04 |
| RS4313076 | 3 | | | 7 | 9351828 | ‘A/C’ | 1.17 | 9.72E−04 |
| RS688630 | 3 | TCTEX1D1 | 200132 | 1 | 66995554 | ‘A/G’ | 0.67 | 9.73E−04 |
| RS10810351 | 3 | | | 9 | 1510510 | ‘A/G’ | −0.97 | 9.91E−04 |
| RS9495378 | 3 | | | 6 | 139564988 | ‘A/G’ | −1.03 | 9.96E−04 |
| RS6632802 | 3 | | | X | 16260152 | ‘C/T’ | 0.68 | 9.96E−04 |
| RS6882366 | 3 | | | 5 | 95890449 | ‘C/T’ | −1.03 | 9.98E−04 |
| RS1715843 | 3 | | | 2 | 228511974 | ‘C/T’ | 0.65 | 9.99E−04 |

dbSNP_rs_ID: SNP identification number in NCBI dbSNP database
Gene_locus: Gene locus and gene id as reported by NCBI dbSNP database build 126
Priority_date: SNP listed in 1: US 11/245,248 2: US 60/819,014 3: US 60/867,454
Sequence_ID: Sequence identification number
Position: Basepair Position, SNP physical position according to NCBI Human Genome Build 36.1
Variants: Alternate SNP alleles or their complementary nucleotides in the position indicated by dbSNP RS ID and basepair position
Minor Allele: SNP allele or its complementary nucleotide that is less common in the control population.
Allele_x2: Chi-squared test based on allele frequencies
Coefficient: Coefficient w of the model glm(z~w + r, family = binomial (link = logit)) in R where z is hypertension status, w is genotype (0, 0<--1, 1<--2) and r is T2D status
P value: P value of the coefficient w
Gene_content: Genes positioned within 100 Kbp up and downstream from the physical position of the SNPs based on NCBI Human Genome Build 36.1

TABLE 9
Haplotype genomic regions with the strongest association
with hypertension in the haplotype sharing analysis (HaploRec + HPM)
with 5 SNPs. The analysis is based on the combined data of 110 HT
cases and 110 healthy controls from the Ashkenazi Jew population,
114 HT cases and 114 healthy controls from the East Finnish population,
41 HT cases and 41 healthy controls from the German population
and 28 HT cases and 28 healthy controls from the English population.

| | | Gene locus | | | | | |
|---|---|---|---|---|---|---|---|
| | | and | | | | | |
| dbSNP rs ID | Priority date | Gene ID | | Chromosome | Position | Variats | P value |
| RS2122952 | 3 | | | 2 | 195004717 | ‘A/G’ | 0.0002 |
| RS2060798 | 3 | | | 2 | 195008975 | ‘G/T’ | <0.0001 |
| RS1451703 | 3 | | | 2 | 195009566 | ‘A/G’ | 0.0009 |
| RS2128663 | 3 | ZPBP | 11055 | 7 | 50023480 | ‘C/T’ | 0.0008 |
| RS959678 | 3 | ZPBP | 11055 | 7 | 50031156 | ‘A/G’ | <0.0001 |
| RS1384634 | 3 | ZPBP | 11055 | 7 | 50035023 | ‘C/T’ | <0.0001 |
| RS12718237 | 3 | ZPBP | 11055 | 7 | 50081500 | ‘C/T’ | <0.0001 |
| RS1870029 | 3 | LOC392670 | 392670 | 7 | 50114262 | ‘C/T’ | 0.0005 |
| RS2249963 | 3 | | | 8 | 11512635 | ‘C/T’ | <0.0001 |
| RS1017804 | 3 | | | 8 | 11515365 | ‘A/C’ | 0.0003 |
| RS7028628 | 3 | | | 9 | 137223172 | ‘G/T’ | 0.0006 |
| RS1891999 | 3 | | | 9 | 137226410 | ‘G/T’ | 0.0001 |
| RS11794621 | 3 | | | 9 | 137231415 | ‘C/T’ | <0.0001 |
| RS1891996 | 3 | | | 9 | 137233596 | ‘A/G’ | 0.0003 |
| RS10506851 | 3 | PPFIA2 | 8499 | 12 | 80662290 | ‘A/G’ | <0.0001 |
| RS4519318 | 3 | | | 15 | 96252471 | ‘A/G’ | <0.0001 |
| RS1075440 | 3 | FTO | 79068 | 16 | 52348407 | ‘A/G’ | 0.0003 |
| RS8050136 | 3 | FTO | 79068 | 16 | 52373776 | ‘A/C’ | <0.0001 |
| RS3751812 | 3 | FTO | 79068 | 16 | 52375961 | ‘G/T’ | 0.0002 |
| RS9319757 | 3 | | | 18 | 64003719 | ‘C/T’ | <0.0001 |
| RS8139003 | 3 | | | 22 | 47590619 | ‘A/G’ | 0.0007 |
| RS7288568 | 3 | LOC648551 | 648551 | 22 | 47595366 | ‘C/T’ | <0.0001 |
| RS1531812 | 3 | | | X | 5712016 | ‘C/T’ | 0.0004 |
| RS5961851 | 3 | | | X | 5722793 | ‘A/G’ | <0.0001 |
| RS5961861 | 3 | | | X | 5738176 | ‘C/T’ | <0.0001 |
| RS6529882 | 3 | | | X | 5746495 | ‘A/G’ | <0.0001 |
| RS5961868 | 3 | | | X | 5750117 | ‘G/T’ | <0.0001 |
| RS3788776 | 3 | ODZ1 | 10178 | X | 123512044 | ‘A/G’ | <0.0001 |
| RS2843518 | 3 | ODZ1 | 10178 | X | 123515881 | ‘C/T’ | 0.0003 |
| RS11260476 | 3 | ODZ1 | 10178 | X | 123517696 | ‘C/T’ | <0.0001 |
| RS2858438 | 3 | ODZ1 | 10178 | X | 123529425 | ‘C/T’ | 0.0004 |
| RS2283740 | 3 | CXorf6 | 10046 | X | 149387455 | ‘C/T’ | 0.0005 |
| RS2073043 | 3 | CXorf6 | 10046 | X | 149392677 | ‘A/G’ | <0.0001 |
| RS547771 | 3 | CXorf6 | 10046 | X | 149394072 | ‘A/G’ | <0.0001 |
| RS731426 | 3 | CXorf6 | 10046 | X | 149395757 | ‘C/T’ | <0.0001 |
| RS523773 | 3 | CXorf6 | 10046 | X | 149396625 | ‘A/G’ | <0.0001 |
| RS477252 | 3 | CXorf6 | 10046 | X | 149398941 | ‘A/G’ | 0.0001 |
| RS10915318 | 3 | | | 1 | 4004999 | ‘A/G’ | 0.0001 |
| RS12749761 | 3 | | | 1 | 4011047 | ‘A/G’ | 0.0004 |
| RS3820742 | 3 | ACVR1 | 90 | 2 | 158344587 | ‘C/T’ | 0.0001 |
| RS10497190 | 1 | ACVR1 | 90 | 2 | 158347486 | ‘C/T’ | 0.0006 |
| RS2160871 | 3 | ATP2B2 | 491 | 3 | 10421826 | ‘A/G’ | 0.0006 |
| RS34904 | 3 | ATP2B2 | 491 | 3 | 10426267 | ‘A/G’ | 0.0001 |
| RS34914 | 3 | ATP2B2 | 491 | 3 | 10432314 | ‘A/G’ | 0.0001 |
| RS11719939 | 3 | ATP2B2 | 491 | 3 | 10432572 | ‘A/G’ | 0.0003 |
| RS9849596 | 3 | | | 3 | 79839174 | ‘A/G’ | 0.0001 |
| RS9309828 | 3 | | | 3 | 79843464 | ‘A/G’ | 0.0001 |
| RS6803083 | 3 | | | 3 | 79852274 | ‘G/T’ | 0.0002 |
| RS6548651 | 3 | | | 3 | 79867106 | ‘A/G’ | 0.0005 |
| RS7639547 | 3 | | | 3 | 104505765 | ‘A/G’ | 0.0001 |
| RS159977 | 3 | | | 5 | 152708309 | ‘A/C’ | 0.0001 |
| RS159978 | 3 | | | 5 | 152717768 | ‘A/G’ | 0.0006 |
| RS1337420 | 3 | GRIK2 | 2898 | 6 | 102203016 | ‘C/T’ | 0.0001 |
| RS12193068 | 3 | GRIK2 | 2898 | 6 | 102208930 | ‘A/C’ | 0.0006 |
| RS217510 | 3 | CREB5 | 9586 | 7 | 28484580 | ‘C/T’ | 0.0007 |
| RS217517 | 3 | CREB5 | 9586 | 7 | 28488170 | ‘A/G’ | 0.0001 |
| RS6988809 | 3 | | | 8 | 40925672 | ‘C/T’ | 0.0006 |
| RS17571033 | 3 | | | 8 | 40929479 | ‘A/G’ | 0.0001 |
| RS884540 | 3 | | | 9 | 8250902 | ‘A/G’ | 0.0001 |
| RS1027584 | 3 | | | 9 | 8258051 | ‘A/G’ | 0.0009 |
| RS7910196 | 3 | FRMD4A | 55691 | 10 | 13850065 | ‘A/G’ | 0.0004 |
| RS2049745 | 3 | FRMD4A | 55691 | 10 | 13857950 | ‘A/G’ | 0.0001 |
| RS2042707 | 3 | | | 13 | 22146799 | ‘C/T’ | 0.0001 |
| RS9506903 | 3 | | | 13 | 22150146 | ‘C/T’ | 0.0003 |
| RS2258026 | 3 | ABR | 29 | 17 | 957228 | ‘A/G’ | 0.001 |
| RS7406978 | 3 | ABR | 29 | 17 | 983909 | ‘C/T’ | 0.0001 |
| RS2440766 | 3 | ABR | 29 | 17 | 989709 | ‘A/G’ | 0.0003 |
| RS7207116 | 3 | ABR | 29 | 17 | 1015143 | ‘A/G’ | 0.0005 |
| RS11655015 | 3 | ABR | 29 | 17 | 1018374 | ‘C/T’ | 0.0009 |
| RS11088668 | 3 | | | 21 | 18448920 | ‘C/T’ | 0.0001 |
| RS2824664 | 3 | | | 21 | 18450015 | ‘C/T’ | 0.0001 |
| RS2284006 | 3 | CACNG2 | 10369 | 22 | 35399975 | ‘C/T’ | 0.0001 |
| RS3850163 | 3 | | | X | 28355558 | ‘G/T’ | 0.0001 |
| RS878007 | 3 | DMD | 1756 | X | 31760187 | ‘A/G’ | 0.0001 |
| RS206061 | 3 | | | X | 41841584 | ‘A/G’ | 0.0006 |
| RS432284 | 3 | | | X | 41846701 | ‘A/G’ | 0.0003 |
| RS206056 | 3 | | | X | 41846775 | ‘C/T’ | 0.0001 |
| RS5918294 | 3 | | | X | 41854429 | ‘C/T’ | 0.0001 |
| RS12853682 | 3 | CXorf43 | 139324 | X | 83570446 | ‘C/T’ | 0.0001 |
| RS5924105 | 3 | | | X | 86889097 | ‘A/G’ | 0.001 |
| RS12557304 | 3 | | | X | 86910552 | ‘C/T’ | 0.0001 |
| RS2208908 | 3 | | | X | 86915504 | ‘A/G’ | 0.0003 |
| RS7051454 | 3 | | | X | 120029242 | ‘A/C’ | 0.0001 |
| RS596987 | 3 | | | X | 144193420 | ‘A/G’ | 0.0001 |
| RS580628 | 3 | | | X | 144250244 | ‘A/C’ | 0.0002 |
| RS481091 | 3 | | | X | 144251889 | ‘A/G’ | 0.0003 |
| RS995895 | 3 | | | X | 144258291 | ‘A/G’ | 0.0001 |
| RS13100475 | 3 | | | 3 | 192637284 | ‘A/G’ | 0.0002 |
| RS4863179 | 3 | | | 4 | 190635394 | ‘A/C’ | 0.0002 |
| RS386936 | 3 | LOC442237 | 442237 | 6 | 97264417 | ‘C/T’ | 0.0002 |
| RS1334327 | 3 | LOC442237 | 442237 | 6 | 97269908 | ‘A/G’ | 0.001 |
| RS38557 | 3 | CACNA2D1 | 781 | 7 | 81720845 | ‘A/G’ | 0.0002 |
| RS4518686 | 3 | | | 8 | 126539999 | ‘A/G’ | 0.0002 |
| RS10490913 | 3 | | | 10 | 120144426 | ‘C/T’ | 0.0004 |
| RS853925 | 3 | | | 10 | 120144856 | ‘A/G’ | 0.0005 |
| RS853943 | 3 | | | 10 | 120154241 | ‘C/T’ | 0.0004 |
| RS10886243 | 3 | | | 10 | 120171437 | ‘G/T’ | 0.0002 |
| RS1013620 | 3 | | | 10 | 120177712 | ‘A/G’ | 0.0002 |
| RS10886244 | 3 | | | 10 | 120182316 | ‘A/G’ | 0.0007 |
| RS220838 | 3 | IGSF4 | 23705 | 11 | 114819312 | ‘A/G’ | 0.0005 |
| RS314474 | 3 | IGSF4 | 23705 | 11 | 114826343 | ‘A/G’ | 0.0005 |
| RS10502202 | 3 | IGSF4 | 23705 | 11 | 114829700 | ‘A/G’ | 0.0002 |
| RS10047420 | 3 | IGSF4 | 23705 | 11 | 114834362 | ‘A/G’ | 0.0002 |
| RS10891859 | 3 | IGSF4 | 23705 | 11 | 114840831 | ‘A/G’ | 0.0002 |
| RS314494 | 3 | IGSF4 | 23705 | 11 | 114841812 | ‘A/G’ | 0.0003 |
| RS7983414 | 3 | | | 13 | 26355842 | ‘A/G’ | 0.0005 |
| RS7994792 | 3 | | | 13 | 26356054 | ‘G/T’ | 0.0002 |
| RS1950771 | 3 | | | 14 | 94334655 | ‘A/G’ | 0.0002 |
| RS10136233 | 3 | | | 14 | 94334883 | ‘A/G’ | 0.0002 |
| RS2691239 | 3 | | | 19 | 56243624 | ‘A/G’ | 0.0006 |
| RS1880413 | 3 | KLK13 | 26085 | 19 | 56252279 | ‘C/T’ | 0.0002 |
| RS7059234 | 3 | | | X | 24948077 | ‘C/T’ | 0.001 |
| RS5986723 | 3 | | | X | 24965806 | ‘A/G’ | 0.0002 |
| RS2188616 | 3 | | | X | 24975597 | ‘C/T’ | 0.0003 |
| RS6673711 | 3 | LOC126917 | 126917 | 1 | 19108104 | ‘C/T’ | 0.0003 |
| RS1513089 | 2 | LOC642216 | 642216 | 5 | 17965365 | ‘A/C’ | 0.0003 |
| RS7014552 | 3 | TSNARE1 | 203062 | 8 | 143408330 | ‘A/G’ | 0.0003 |
| RS3858054 | 3 | | | 9 | 8243589 | ‘C/T’ | 0.0003 |
| RS10976882 | 3 | | | 9 | 8247948 | ‘A/G’ | 0.0005 |
| RS2167644 | 3 | LRRN6C | 158038 | 9 | 28076344 | ‘A/G’ | 0.0005 |
| RS10968337 | 3 | LRRN6C | 158038 | 9 | 28076683 | ‘C/T’ | 0.0003 |
| RS4305993 | 3 | LRRN6C | 158038 | 9 | 28078526 | ‘C/T’ | 0.0003 |
| RS4611181 | 3 | ZNF195 | 7748 | 11 | 3349321 | ‘C/T’ | 0.0003 |
| RS1150935 | 3 | | | 12 | 27493452 | ‘C/T’ | 0.0003 |
| RS306594 | 3 | | | 12 | 27500523 | ‘G/T’ | 0.0005 |
| RS1000703 | 3 | | | 12 | 93780350 | ‘A/G’ | 0.0008 |
| RS892492 | 3 | | | 12 | 93784834 | ‘C/T’ | 0.0005 |
| RS7961204 | 3 | | | 12 | 93801511 | ‘C/T’ | 0.0003 |
| RS10777647 | 3 | | | 12 | 93804844 | ‘C/T’ | 0.0003 |
| RS12588192 | 3 | MAMDC1 | 161357 | 14 | 46651013 | ‘G/T’ | 0.0003 |
| RS8037284 | 3 | | | 15 | 58176069 | ‘C/T’ | 0.0009 |
| RS10048054 | 3 | | | 15 | 58185128 | ‘C/T’ | 0.0003 |
| RS8063120 | 3 | | | 16 | 80352553 | ‘A/G’ | 0.0003 |
| RS10445097 | 3 | | | 16 | 80357386 | ‘G/T’ | 0.0009 |
| RS873634 | 3 | HCN2 | 610 | 19 | 539305 | ‘G/T’ | 0.0003 |
| RS2047373 | 3 | DMD | 1756 | X | 31676566 | ‘C/T’ | 0.0003 |
| RS2061426 | 3 | | | X | 124373041 | ‘A/G’ | 0.0003 |
| RS5904833 | 3 | FMR1NB | 158521 | X | 146892840 | ‘A/G’ | 0.0003 |
| RS764631 | 3 | FMR1NB | 158521 | X | 146895941 | ‘C/T’ | 0.0003 |
| RS12499689 | 3 | | | 4 | 13879456 | ‘C/T’ | 0.0005 |
| RS3111813 | 3 | | | 4 | 13884425 | ‘A/C’ | 0.0004 |
| RS7034341 | 3 | SUSD1 | 64420 | 9 | 113854649 | ‘C/T’ | 0.0004 |
| RS220836 | 3 | IGSF4 | 23705 | 11 | 114807081 | ‘A/G’ | 0.0004 |
| RS10488707 | 3 | IGSF4 | 23705 | 11 | 114807162 | ‘A/G’ | 0.0007 |
| RS1749704 | 2 | TTC7B | 145567 | 14 | 90239107 | ‘G/T’ | 0.0007 |
| RS1535321 | 2 | TTC7B | 145567 | 14 | 90240579 | ‘C/T’ | 0.0004 |
| RS1749718 | 2 | TTC7B | 145567 | 14 | 90253080 | ‘C/T’ | 0.0005 |
| RS1742083 | 2 | TTC7B | 145567 | 14 | 90256423 | ‘C/T’ | 0.0008 |
| RS8047519 | 3 | | | 16 | 49026539 | ‘C/T’ | 0.0004 |
| RS1548914 | 3 | | | 16 | 49036872 | ‘A/C’ | 0.0006 |
| RS1232143 | 3 | | | X | 8798982 | ‘A/G’ | 0.0004 |
| RS1458368 | 3 | DMD | 1756 | X | 31730435 | ‘A/G’ | 0.0004 |
| RS5928121 | 3 | DMD | 1756 | X | 32862842 | ‘A/G’ | 0.0004 |
| RS5931268 | 2 | | | X | 136893265 | ‘G/T’ | 0.0004 |
| RS962429 | 3 | C1orf125 | 126859 | 1 | 177719318 | ‘C/T’ | 0.0005 |
| RS3748971 | 3 | ECEL1P2 | 347694 | 2 | 232958927 | ‘A/G’ | 0.0005 |
| RS1873038 | 3 | NLGN1 | 22871 | 3 | 175020335 | ‘A/G’ | 0.0005 |
| RS13105217 | 3 | | | 4 | 65064629 | ‘C/T’ | 0.0005 |
| RS10962912 | 3 | | | 9 | 17209242 | ‘C/T’ | 0.0005 |
| RS11046835 | 3 | | | 12 | 23211061 | ‘C/T’ | 0.0005 |
| RS1870943 | 3 | | | 12 | 88192283 | ‘C/T’ | 0.0005 |
| RS9936750 | 3 | | | 16 | 53729375 | ‘C/T’ | 0.0005 |
| RS1486735 | 3 | | | 16 | 53737593 | ‘A/G’ | 0.0009 |
| RS8072734 | 3 | | | 17 | 14642128 | ‘A/G’ | 0.0005 |
| RS9622650 | 3 | | | 22 | 36315673 | ‘C/T’ | 0.0005 |
| RS7291493 | 3 | | | 22 | 47615371 | ‘A/G’ | 0.0005 |
| RS812452 | 3 | | | X | 7123308 | ‘C/T’ | 0.0005 |
| RS12861247 | 3 | STS | 412 | X | 7184199 | ‘A/G’ | 0.0009 |
| RS4825236 | 3 | PPEF1 | 5475 | X | 18642674 | ‘C/T’ | 0.0008 |
| RS2269584 | 3 | PPEF1 | 5475 | X | 18689642 | ‘A/G’ | 0.0007 |
| RS2269586 | 3 | PPEF1 | 5475 | X | 18690101 | ‘A/G’ | 0.0005 |
| RS5925675 | 3 | | | X | 22742435 | ‘A/G’ | 0.0005 |
| RS1935074 | 3 | | | X | 80063759 | ‘C/T’ | 0.0005 |
| RS10482585 | 3 | LOC648118 | 648118 | X | 80073549 | ‘C/T’ | 0.0007 |
| RS592807 | 2 | GRIA3 | 2892 | X | 122419337 | ‘C/T’ | 0.0006 |
| RS503118 | 2 | GRIA3 | 2892 | X | 122421904 | ‘C/T’ | 0.0005 |
| RS585602 | 2 | | | X | 137025064 | ‘A/G’ | 0.0005 |
| RS5919819 | 3 | | | X | 144161129 | ‘G/T’ | 0.0005 |
| RS1488547 | 3 | NLGN1 | 22871 | 3 | 175008462 | ‘C/T’ | 0.0006 |
| RS9290481 | 3 | NLGN1 | 22871 | 3 | 175013923 | ‘A/G’ | 0.0009 |
| RS9296444 | 3 | | | 6 | 44728485 | ‘C/T’ | 0.0008 |
| RS1021129 | 3 | | | 6 | 44735522 | ‘C/T’ | 0.0006 |
| RS1377050 | 3 | LRRN6C | 158038 | 9 | 28090836 | ‘A/G’ | 0.0006 |
| RS1416836 | 3 | | | 9 | 109806784 | ‘C/T’ | 0.0009 |
| RS9695286 | 3 | | | 9 | 109812888 | ‘C/T’ | 0.0006 |
| RS1747839 | 3 | LOC650079 | 650079 | 9 | 138155232 | ‘G/T’ | 0.0006 |
| RS2767431 | 3 | LOC647525 | 647525 | 10 | 134472497 | ‘C/T’ | 0.0006 |
| RS2387069 | 3 | C10orf92 | 54777 | 10 | 134475893 | ‘A/G’ | 0.0006 |
| RS12818362 | 3 | | | 12 | 92915037 | ‘C/T’ | 0.0006 |
| RS984429 | 3 | | | 18 | 47941339 | ‘A/C’ | 0.0006 |
| RS2837705 | 3 | DSCAM | 1826 | 21 | 40852671 | ‘C/T’ | 0.0007 |
| RS2837709 | 3 | DSCAM | 1826 | 21 | 40861610 | ‘A/C’ | 0.0006 |
| RS2837713 | 3 | DSCAM | 1826 | 21 | 40873626 | ‘A/C’ | 0.001 |
| RS2837716 | 3 | DSCAM | 1826 | 21 | 40875564 | ‘A/G’ | 0.001 |
| RS2007215 | 3 | PTCHD2 | 57540 | 1 | 11460564 | ‘A/G’ | 0.0007 |
| RS4846012 | 3 | PTCHD2 | 57540 | 1 | 11480513 | ‘G/T’ | 0.0007 |
| RS2053671 | 3 | KCNJ3 | 3760 | 2 | 155345484 | ‘G/T’ | 0.0007 |
| RS6433574 | 3 | | | 2 | 176806772 | ‘A/G’ | 0.0007 |
| RS6816464 | 3 | | | 4 | 19537700 | ‘C/T’ | 0.0007 |
| RS4464568 | 3 | | | 4 | 19542167 | ‘C/T’ | 0.0007 |
| RS2086735 | 3 | | | 4 | 65006948 | ‘G/T’ | 0.0007 |
| RS4865755 | 3 | ITGA2 | 3673 | 5 | 52326944 | ‘C/T’ | 0.0007 |
| RS17237251 | 3 | | | 5 | 67447959 | ‘C/T’ | 0.0007 |
| RS2888306 | 3 | | | 5 | 67448240 | ‘C/T’ | 0.001 |
| RS6890771 | 3 | | | 5 | 180014372 | ‘C/T’ | 0.001 |
| RS7705017 | 3 | | | 5 | 180026648 | ‘C/T’ | 0.0007 |
| RS9405675 | 3 | | | 6 | 389600 | ‘A/G’ | 0.0007 |
| RS7755154 | 3 | | | 6 | 2558943 | ‘C/T’ | 0.0007 |
| RS10215999 | 3 | | | 7 | 13640063 | ‘C/T’ | 0.0007 |
| RS10513402 | 1 | | | 9 | 124204757 | ‘C/T’ | 0.0007 |
| RS10899922 | 3 | C10orf136 | 414260 | 10 | 43661970 | ‘A/G’ | 0.0007 |
| RS1463632 | 3 | | | 16 | 53741402 | ‘A/G’ | 0.0007 |
| RS1734920 | 3 | | | 21 | 40266781 | ‘G/T’ | 0.0007 |
| RS8131179 | 3 | PDE9A | 5152 | 21 | 42955270 | ‘C/T’ | 0.0009 |
| RS2284958 | 3 | PDE9A | 5152 | 21 | 42961700 | ‘C/T’ | 0.0007 |
| RS373747 | 3 | | | 22 | 18535192 | ‘C/T’ | 0.0007 |
| RS5955922 | 3 | | | X | 17970012 | ‘C/T’ | 0.001 |
| RS6527831 | 3 | | | X | 18015188 | ‘A/C’ | 0.0007 |
| RS5955936 | 3 | | | X | 18046471 | ‘A/G’ | 0.0008 |
| RS2050909 | 3 | | | X | 137313831 | ‘A/G’ | 0.0007 |
| RS10187702 | 3 | LOC652214 | 652214 | 2 | 58723279 | ‘C/T’ | 0.0008 |
| RS7591633 | 3 | LOC400955 | 400955 | 2 | 58725562 | ‘A/G’ | 0.0008 |
| RS6715162 | 3 | LOC400955 | 400955 | 2 | 58740811 | ‘C/T’ | 0.0009 |
| RS2166512 | 3 | | | 2 | 176779427 | ‘A/G’ | 0.0008 |
| RS6931600 | 3 | | | 6 | 130140464 | ‘C/T’ | 0.0008 |
| RS1871400 | 3 | | | 6 | 153580911 | ‘A/G’ | 0.0008 |
| RS2732744 | 3 | | | 7 | 84680820 | ‘A/G’ | 0.0008 |
| RS4242499 | 3 | | | 8 | 4867155 | ‘G/T’ | 0.0008 |
| RS6990880 | 3 | | | 8 | 4868194 | ‘C/T’ | 0.0008 |
| RS4873814 | 3 | | | 8 | 144793335 | ‘A/G’ | 0.0008 |
| RS716064 | 3 | NRXN3 | 9369 | 14 | 78753709 | ‘A/C’ | 0.0008 |
| RS10853007 | 3 | GJA7 | 10052 | 17 | 40248321 | ‘A/G’ | 0.001 |
| RS1071682 | 3 | HIGD1B | 51751 | 17 | 40283247 | ‘C/T’ | 0.0008 |
| RS2267064 | 3 | LOC648941 | 648941 | 22 | 22874632 | ‘G/T’ | 0.0008 |
| RS5927001 | 3 | DMD | 1756 | X | 31161215 | ‘C/T’ | 0.0008 |
| RS5953392 | 3 | | | X | 44137384 | ‘A/G’ | 0.0008 |
| RS1831116 | 3 | | | X | 83811301 | ‘C/T’ | 0.0008 |
| RS17036947 | 3 | PTCHD2 | 57540 | 1 | 11497847 | ‘G/T’ | 0.001 |
| RS2072996 | 3 | PTCHD2 | 57540 | 1 | 11501560 | ‘A/G’ | 0.0009 |
| RS2817632 | 3 | PTCHD2 | 57540 | 1 | 11510818 | ‘A/G’ | 0.0009 |
| RS2076468 | 3 | PTCHD2 | 57540 | 1 | 11512498 | ‘C/T’ | 0.001 |
| RS561264 | 3 | | | 2 | 238994718 | ‘A/C’ | 0.0009 |
| RS11128372 | 3 | | | 3 | 74096334 | ‘A/G’ | 0.0009 |
| RS4315784 | 3 | | | 4 | 19551691 | ‘A/G’ | 0.0009 |
| RS6826691 | 3 | | | 4 | 164821659 | ‘C/T’ | 0.0009 |
| RS6897616 | 3 | LOC642216 | 642216 | 5 | 17884426 | ‘A/G’ | 0.0009 |
| RS2607605 | 3 | | | 8 | 24700639 | ‘C/T’ | 0.0009 |
| RS1879188 | 2 | PTK2B | 2185 | 8 | 27249840 | ‘G/T’ | 0.0009 |
| RS10283134 | 3 | C8orf36 | 286053 | 8 | 126341828 | ‘A/G’ | 0.0009 |
| RS10773557 | 3 | | | 12 | 127638497 | ‘A/C’ | 0.0009 |
| RS1926005 | 3 | | | 13 | 45731151 | ‘A/C’ | 0.0009 |
| RS2239975 | 3 | SYT17 | 51760 | 16 | 19104701 | ‘G/T’ | 0.0009 |
| RS12450029 | 3 | ABR | 29 | 17 | 949596 | ‘C/T’ | 0.0009 |
| RS5905269 | 2 | | | X | 115402180 | ‘A/C’ | 0.0009 |
| RS1293468 | 3 | | | X | 122036209 | ‘C/T’ | 0.0009 |
| RS644210 | 3 | SPANX-N1 | 494118 | X | 144142532 | ‘G/T’ | 0.0009 |
| RS150571 | 3 | | | 1 | 37430550 | ‘C/T’ | 0.001 |
| RS803441 | 3 | | | 1 | 162450455 | ‘C/T’ | 0.001 |
| RS2881507 | 3 | | | 1 | 162492876 | ‘A/G’ | 0.001 |
| RS12133017 | 3 | C1orf125 | 126859 | 1 | 177708960 | ‘C/T’ | 0.001 |
| RS4686599 | 2 | | | 3 | 193327806 | ‘C/T’ | 0.001 |
| RS1511776 | 3 | | | 4 | 164801907 | ‘C/T’ | 0.001 |
| RS2391671 | 3 | CREB5 | 9586 | 7 | 28518902 | ‘A/G’ | 0.001 |
| RS2284218 | 3 | CRHR2 | 1395 | 7 | 30680858 | ‘C/T’ | 0.001 |
| RS2245192 | 3 | | | 7 | 113789771 | ‘C/T’ | 0.001 |
| RS419490 | 3 | | | 9 | 106836369 | ‘A/C’ | 0.001 |
| RS888219 | 3 | | | 9 | 127968844 | ‘G/T’ | 0.001 |
| RS7644 | 3 | PARVA | 55742 | 11 | 12508420 | ‘C/T’ | 0.001 |
| RS10892358 | 3 | | | 11 | 118761005 | ‘A/G’ | 0.001 |
| RS1793566 | 3 | | | 11 | 130636689 | ‘C/T’ | 0.001 |
| RS9506776 | 3 | LOC650912 | 650912 | 13 | 21518850 | ‘C/T’ | 0.001 |
| RS1561942 | 3 | AK7 | 122481 | 14 | 95955212 | ‘A/G’ | 0.001 |
| RS8015440 | 3 | AK7 | 122481 | 14 | 95961302 | ‘A/C’ | 0.001 |
| RS2068259 | 3 | C20orf74 | 57186 | 20 | 20341219 | ‘A/C’ | 0.001 |
| RS133519 | 2 | | | 22 | 46955392 | ‘G/T’ | 0.001 |
| RS1999925 | 3 | | | X | 93215057 | ‘A/G’ | 0.001 |
| RS5949848 | 3 | | | X | 95623109 | ‘C/T’ | 0.001 |

dbSNP_rs_ID: SNP identification number in NCBI dbSNP database
Sequence_ID: Sequence identification number
Priority_date: SNP listed in 1: US 11/245,248 2: US 60/819,014 3: US 60/867,454
Gene_locus: Gene locus and gene id as reported by NCBI dbSNP database build 126
Variants: Alternate SNP alleles or their complementary nucleotides in the position indicated by dbSNP RS ID and basepair position
P-value: P-value based on permutation test
Position: Basepair Position, SNP physical position according to NCBI Human Genome Build 36.1
Gene_content: Genes positioned within 100 Kbp up and downstream (End) from the physical position of the SNPs bordering the haplotype genomic region based on NCBI Human Genome Build 36.

TABLE 10
Haplotypes with the strongest association with hypertension
based on HaploRec + HPM analysis with 5 SNPs. The analysis is based
on the combined data of 110 HT cases and 110 healthy controls from
the Ashkenazi Jew population, 114 HT cases and 114 healthy controls
from the East Finnish population, 41 HT cases and 41 healthy controls
from the German population and 28 HT cases and 28 healthy controls
from the English population.

| | | Gene locus | | | | | | |
|---|---|---|---|---|---|---|---|---|
| | | and | | | | | | |
| dbSNP rs ID | Priority date | Gene ID | | Chromosome | Position | Variats | Risk Allele | Chi square |
| RS9564765 | 3 | | | 13 | 70431786 | ‘A/G’ | G | 22.83 |
| RS803815 | 3 | | | 13 | 70434610 | ‘C/T’ | G | |
| RS2202564 | 3 | | | 13 | 70430344 | ‘A/G’ | A | 22.25 |
| RS9564765 | 3 | | | 13 | 70431786 | ‘A/G’ | G | |
| RS803815 | 3 | | | 13 | 70434610 | ‘C/T’ | G | |
| RS2265326 | 3 | | | 13 | 64959554 | ‘C/T’ | A | 21.68 |
| RS2067741 | 3 | | | 13 | 64966931 | ‘A/G’ | A | |
| RS9540461 | 3 | | | 13 | 64989389 | ‘A/G’ | G | |
| RS9540464 | 3 | | | 13 | 65000270 | ‘A/G’ | A | |
| RS9598990 | 3 | | | 13 | 65001632 | ‘A/C’ | A | |
| RS950942 | 3 | | | 13 | 70429810 | ‘C/T’ | A | 21.67 |
| RS2202564 | 3 | | | 13 | 70430344 | ‘A/G’ | A | |
| RS9564765 | 3 | | | 13 | 70431786 | ‘A/G’ | G | |
| RS803815 | 3 | | | 13 | 70434610 | ‘C/T’ | G | |
| RS2067741 | 3 | | | 13 | 64966931 | ‘A/G’ | A | 21.40 |
| RS9540461 | 3 | | | 13 | 64989389 | ‘A/G’ | G | |
| RS9540464 | 3 | | | 13 | 65000270 | ‘A/G’ | A | |
| RS9598990 | 3 | | | 13 | 65001632 | ‘A/C’ | A | |
| RS9571419 | 3 | | | 13 | 65001891 | ‘A/C’ | C | |
| RS2067741 | 3 | | | 13 | 64966931 | ‘A/G’ | A | 21.40 |
| RS9540461 | 3 | | | 13 | 64989389 | ‘A/G’ | G | |
| RS9540464 | 3 | | | 13 | 65000270 | ‘A/G’ | A | |
| RS9598990 | 3 | | | 13 | 65001632 | ‘A/C’ | A | |
| RS2067741 | 3 | | | 13 | 64966931 | ‘A/G’ | A | 20.82 |
| RS9540461 | 3 | | | 13 | 64989389 | ‘A/G’ | G | |
| RS9540464 | 3 | | | 13 | 65000270 | ‘A/G’ | A | |
| RS9317509 | 3 | | | 13 | 64943251 | ‘C/T’ | A | 19.89 |
| RS2265326 | 3 | | | 13 | 64959554 | ‘C/T’ | A | |
| RS2067741 | 3 | | | 13 | 64966931 | ‘A/G’ | A | |
| RS9540461 | 3 | | | 13 | 64989389 | ‘A/G’ | G | |
| RS9540464 | 3 | | | 13 | 65000270 | ‘A/G’ | A | |
| RS2806939 | 3 | | | 13 | 52648971 | ‘C/T’ | G | 19.88 |
| RS2806947 | 3 | | | 13 | 52666026 | ‘A/G’ | A | |
| RS1322949 | 3 | LOC647339 | 647339 | 13 | 52671659 | ‘C/T’ | G | |
| RS2806957 | 3 | | | 13 | 52688480 | ‘C/T’ | G | |
| RS1923773 | 3 | | | 13 | 52648355 | ‘C/T’ | A | 19.88 |
| RS2806939 | 3 | | | 13 | 52648971 | ‘C/T’ | G | |
| RS2806947 | 3 | | | 13 | 52666026 | ‘A/G’ | A | |
| RS1322949 | 3 | LOC647339 | 647339 | 13 | 52671659 | ‘C/T’ | G | |
| RS2806957 | 3 | | | 13 | 52688480 | ‘C/T’ | G | |
| RS2806947 | 3 | | | 13 | 52666026 | ‘A/G’ | A | 19.82 |
| RS1322949 | 3 | LOC647339 | 647339 | 13 | 52671659 | ‘C/T’ | G | |
| RS2806957 | 3 | | | 13 | 52688480 | ‘C/T’ | G | |
| RS2265326 | 3 | | | 13 | 64959554 | ‘C/T’ | A | 19.77 |
| RS2067741 | 3 | | | 13 | 64966931 | ‘A/G’ | A | |
| RS9540461 | 3 | | | 13 | 64989389 | ‘A/G’ | G | |
| RS9540464 | 3 | | | 13 | 65000270 | ‘A/G’ | A | |
| RS1849067 | 3 | | | 2 | 195675318 | ‘A/G’ | G | 20.71 |
| RS715200 | 3 | | | 2 | 195677764 | ‘A/G’ | G | |
| RS1599755 | 3 | | | 2 | 195682608 | ‘A/G’ | G | |
| RS4047462 | 3 | | | 2 | 16222221 | ‘A/G’ | A | 20.23 |
| RS7422511 | 3 | | | 2 | 16224996 | ‘A/G’ | G | |
| RS12995942 | 3 | | | 2 | 16226902 | ‘A/G’ | A | |
| RS7559122 | 2 | | | 2 | 16229611 | ‘A/G’ | A | |
| RS7560874 | 2 | | | 2 | 16233209 | ‘A/G’ | G | |
| RS11845875 | 3 | AK7 | 122481 | 14 | 96002882 | ‘A/G’ | A | 20.47 |
| RS3809425 | 3 | PAPOLA | 10914 | 14 | 96055906 | ‘A/G’ | A | |
| RS2274795 | 3 | PAPOLA | 10914 | 14 | 96064435 | ‘C/T’ | A | |
| RS8013517 | 3 | PAPOLA | 10914 | 14 | 96082661 | ‘A/G’ | G | |
| RS11160342 | 3 | | | 14 | 96104107 | ‘C/T’ | A | |
| RS10858385 | 3 | | | 9 | 137335350 | ‘C/T’ | G | 20.49 |
| RS3884535 | 3 | | | 9 | 137337030 | ‘C/T’ | A | |
| RS4842247 | 3 | | | 9 | 137339138 | ‘A/G’ | G | |
| RS4775234 | 3 | | | 15 | 58117938 | ‘A/C’ | A | 20.33 |
| RS713469 | 3 | | | 15 | 58121920 | ‘A/G’ | G | |
| RS335787 | 3 | | | 15 | 58124590 | ‘A/G’ | G | |
| RS193097 | 3 | LOC651082 | 651082 | 15 | 58134744 | ‘A/G’ | A | |
| RS4775234 | 3 | | | 15 | 58117938 | ‘A/C’ | A | 20.33 |
| RS713469 | 3 | | | 15 | 58121920 | ‘A/G’ | G | |
| RS335787 | 3 | | | 15 | 58124590 | ‘A/G’ | G | |
| RS193097 | 3 | LOC651082 | 651082 | 15 | 58134744 | ‘A/G’ | A | |
| RS6494166 | 3 | | | 15 | 58140408 | ‘A/G’ | G | |
| RS713469 | 3 | | | 15 | 58121920 | ‘A/G’ | G | 20.33 |
| RS335787 | 3 | | | 15 | 58124590 | ‘A/G’ | G | |
| RS193097 | 3 | LOC651082 | 651082 | 15 | 58134744 | ‘A/G’ | A | |
| RS713469 | 3 | | | 15 | 58121920 | ‘A/G’ | G | 20.33 |
| RS335787 | 3 | | | 15 | 58124590 | ‘A/G’ | G | |
| RS193097 | 3 | LOC651082 | 651082 | 15 | 58134744 | ‘A/G’ | A | |
| RS6494166 | 3 | | | 15 | 58140408 | ‘A/G’ | G | |
| RS335787 | 3 | | | 15 | 58124590 | ‘A/G’ | G | 20.03 |
| RS193097 | 3 | LOC651082 | 651082 | 15 | 58134744 | ‘A/G’ | A | |
| RS11084402 | 3 | ZNF579 | 163033 | 19 | 60785177 | ‘C/T’ | G | 19.87 |
| RS693289 | 3 | ZNF524 | 147807 | 19 | 60802848 | ‘A/G’ | G | |
| RS310465 | 3 | LOC388565 | 388565 | 19 | 60815558 | ‘A/G’ | G | |
| RS4750957 | 3 | | | 10 | 130151665 | ‘C/T’ | A | 20.25 |
| RS7096455 | 3 | | | 10 | 130162023 | ‘G/T’ | C | |
| RS7901182 | 3 | | | 10 | 130168169 | ‘A/G’ | G | |
| RS7915794 | 3 | | | 10 | 130179509 | ‘A/G’ | A | |
| RS7893667 | 3 | | | 10 | 130181728 | ‘A/G’ | G | |
| RS11150469 | 3 | | | 16 | 81114803 | ‘C/T’ | A | 22.48 |
| RS7198864 | 3 | | | 16 | 81119203 | ‘C/T’ | G | |
| RS9931462 | 3 | | | 16 | 81119956 | ‘C/T’ | G | |
| RS766291 | 3 | | | 16 | 81125471 | ‘C/T’ | A | |
| RS7406978 | 3 | ABR | 29 | 17 | 983909 | ‘C/T’ | A | 21.30 |
| RS2440766 | 3 | ABR | 29 | 17 | 989709 | ‘A/G’ | A | |
| RS6502048 | 3 | | | 17 | 77598217 | ‘A/G’ | G | 19.56 |
| RS9915228 | 3 | RFNG | 5986 | 17 | 77601176 | ‘A/G’ | A | |
| RS228039 | 3 | PDE9A | 5152 | 21 | 42945262 | ‘C/T’ | G | 22.75 |
| RS2269127 | 3 | PDE9A | 5152 | 21 | 42950306 | ‘A/G’ | G | |
| RS8131179 | 3 | PDE9A | 5152 | 21 | 42955270 | ‘C/T’ | A | |
| RS228038 | 3 | PDE9A | 5152 | 21 | 42945008 | ‘A/G’ | G | 19.75 |
| RS228039 | 3 | PDE9A | 5152 | 21 | 42945262 | ‘C/T’ | G | |
| RS2269127 | 3 | PDE9A | 5152 | 21 | 42950306 | ‘A/G’ | G | |
| RS8131179 | 3 | PDE9A | 5152 | 21 | 42955270 | ‘C/T’ | A | |
| RS2825172 | 3 | | | 21 | 19119089 | ‘A/G’ | A | 19.58 |
| RS2825180 | 3 | | | 21 | 19124203 | ‘C/T’ | A | |
| RS481091 | 3 | | | X | 144251889 | ‘A/G’ | G | 24.81 |
| RS995895 | 3 | | | X | 144258291 | ‘A/G’ | A | |
| RS17244441 | 3 | | | X | 144270169 | ‘C/T’ | A | |
| RS11094472 | 3 | | | X | 144273211 | ‘A/G’ | G | |
| RS481091 | 3 | | | X | 144251889 | ‘A/G’ | G | 24.81 |
| RS995895 | 3 | | | X | 144258291 | ‘A/G’ | A | |
| RS17244441 | 3 | | | X | 144270169 | ‘C/T’ | A | |
| RS481091 | 3 | | | X | 144251889 | ‘A/G’ | G | 24.81 |
| RS995895 | 3 | | | X | 144258291 | ‘A/G’ | A | |
| RS1924476 | 3 | SMARCA1 | 6594 | X | 128441964 | ‘A/G’ | G | 23.20 |
| RS3131274 | 3 | SMARCA1 | 6594 | X | 128486023 | ‘C/T’ | A | |
| RS3118108 | 3 | | | X | 128496148 | ‘G/T’ | A | |
| RS5977112 | 3 | OCRL | 4952 | X | 128545248 | ‘A/G’ | G | |
| RS2071706 | 3 | OCRL | 4952 | X | 128552206 | ‘A/G’ | G | |
| RS5955898 | 3 | RPS6KA3 | 6197 | X | 20185233 | ‘A/G’ | A | 22.55 |
| RS6418738 | 3 | | | X | 20198804 | ‘A/G’ | G | |
| RS5990883 | 3 | | | X | 20241006 | ‘C/T’ | A | |
| RS7886043 | 3 | | | X | 20246948 | ‘A/C’ | C | |
| RS12689240 | 3 | | | X | 20252714 | ‘C/T’ | A | |
| RS549580 | 3 | GRIA3 | 2892 | X | 122405634 | ‘C/T’ | A | 21.88 |
| RS10521721 | 3 | GRIA3 | 2892 | X | 122405854 | ‘C/T’ | A | |
| RS687577 | 3 | GRIA3 | 2892 | X | 122406785 | ‘G/T’ | C | |
| RS625074 | 3 | GRIA3 | 2892 | X | 122403594 | ‘A/G’ | G | 21.88 |
| RS545958 | 3 | GRIA3 | 2892 | X | 122405251 | ‘A/G’ | A | |
| RS549580 | 3 | GRIA3 | 2892 | X | 122405634 | ‘C/T’ | A | |
| RS10521721 | 3 | GRIA3 | 2892 | X | 122405854 | ‘C/T’ | A | |
| RS687577 | 3 | GRIA3 | 2892 | X | 122406785 | ‘G/T’ | C | |
| RS545958 | 3 | GRIA3 | 2892 | X | 122405251 | ‘A/G’ | A | 21.88 |
| RS549580 | 3 | GRIA3 | 2892 | X | 122405634 | ‘C/T’ | A | |
| RS10521721 | 3 | GRIA3 | 2892 | X | 122405854 | ‘C/T’ | A | |
| RS687577 | 3 | GRIA3 | 2892 | X | 122406785 | ‘G/T’ | C | |
| RS5990883 | 3 | | | X | 20241006 | ‘C/T’ | A | 21.86 |
| RS7886043 | 3 | | | X | 20246948 | ‘A/C’ | C | |
| RS12689240 | 3 | | | X | 20252714 | ‘C/T’ | A | |
| RS6653648 | 3 | | | X | 20265327 | ‘C/T’ | G | |
| RS5950381 | 3 | | | X | 20267586 | ‘C/T’ | A | |
| RS1924476 | 3 | SMARCA1 | 6594 | X | 128441964 | ‘A/G’ | G | 21.25 |
| RS3131274 | 3 | SMARCA1 | 6594 | X | 128486023 | ‘C/T’ | A | |
| RS3118108 | 3 | | | X | 128496148 | ‘G/T’ | A | |
| RS5977112 | 3 | OCRL | 4952 | X | 128545248 | ‘A/G’ | G | |
| RS1324150 | 3 | SMARCA1 | 6594 | X | 128412541 | ‘C/T’ | A | 21.25 |
| RS1924476 | 3 | SMARCA1 | 6594 | X | 128441964 | ‘A/G’ | G | |
| RS3131274 | 3 | SMARCA1 | 6594 | X | 128486023 | ‘C/T’ | A | |
| RS3118108 | 3 | | | X | 128496148 | ‘G/T’ | A | |
| RS5977112 | 3 | OCRL | 4952 | X | 128545248 | ‘A/G’ | G | |
| RS580628 | 3 | | | X | 144250244 | ‘A/C’ | C | 21.02 |
| RS481091 | 3 | | | X | 144251889 | ‘A/G’ | G | |
| RS995895 | 3 | | | X | 144258291 | ‘A/G’ | A | |
| RS580628 | 3 | | | X | 144250244 | ‘A/C’ | C | 21.02 |
| RS481091 | 3 | | | X | 144251889 | ‘A/G’ | G | |
| RS995895 | 3 | | | X | 144258291 | ‘A/G’ | A | |
| RS17244441 | 3 | | | X | 144270169 | ‘C/T’ | A | |
| RS11094472 | 3 | | | X | 144273211 | ‘A/G’ | G | |
| RS580628 | 3 | | | X | 144250244 | ‘A/C’ | C | 21.02 |
| RS481091 | 3 | | | X | 144251889 | ‘A/G’ | G | |
| RS995895 | 3 | | | X | 144258291 | ‘A/G’ | A | |
| RS17244441 | 3 | | | X | 144270169 | ‘C/T’ | A | |
| RS9405986 | 3 | | | 6 | 6859406 | ‘C/T’ | A | 20.83 |
| RS2768999 | 3 | | | 6 | 6863125 | ‘C/T’ | A | |
| RS2769006 | 3 | | | 6 | 6868881 | ‘C/T’ | G | |
| RS2876048 | 3 | | | 6 | 6871409 | ‘C/T’ | G | |
| RS1536242 | 3 | | | 6 | 6876009 | ‘C/T’ | G | |
| RS7954232 | 3 | LOC651534 | 651534 | 12 | 91963517 | ‘C/T’ | G | 19.76 |
| RS4760381 | 3 | LOC651534 | 651534 | 12 | 91972190 | ‘A/G’ | G | |
| RS4584620 | 3 | LOC651534 | 651534 | 12 | 91978954 | ‘C/T’ | G | |
| RS1542481 | 3 | LOC651534 | 651534 | 12 | 91982962 | ‘A/G’ | G | |

dbSNP_rs_ID: SNP identification number in NCBI dbSNP database
Sequence_ID: Sequence identification number
Priority date: SNP listed in 1: US 11/245,248 2: US 60/819,014 3: US 60/867,454
Gene_locus: Gene locus and gene id as reported by NCBI dbSNP database build 126
Position: Basepair Position, SNP physical position according to NCBI Human Genome Build 36.1
Variants: Alternate SNP alleles or their complementary nucleotides in the position indicated by dbSNP RS ID and basepair position
Risk_allele: Allele in at-risk haplotype
Chi_square: Chi-squared test based on allele frequencies
P-value: P-value based on the chi-square test

### REFERENCES

American Heart Association. Heart Disease and Stroke Statistics—2004 Update. Dallas, Tex.: American Heart Association; 2003. (http://www.americanheart.org/downloadable/heart/1079736729696HDSStats2004UpdateREV3-19-04.pdf)
Binder A 2007. A review of the genetics of essential hypertension. Curr Opin Cardiol 22:176-184.
Cusi D et al. 1997. Polymorphisms of alpha-adducin and salt sensitivity in patients with essential hypertension. Lancet 349:1353-1357
Eronen L et al. 2004. A Markov chain approach to reconstruction of long haplotypes. Pac Symp Biocomput: 104-115.
Fuentes R 2003. Familial aggregation and tracking of blood pressure, body mass index and serum total cholesterol during childhood. A prospective family study in eastern Finland. Doctoral Dissertation. Kuopio University Publications D. Medical Sciences 296: 139 p. ISBN 951-781-896-3; ISSN 1235-0303.
Guyton A C. 1991. Blood pressure control-special role of the kidneys and body fluids. Science 252:1813-1816.
Hopkins P N, Hunt S C. 2003. Genetics of hypertension. Genet Med 5:413-429.
Hunt S C, Cook N R, Oberman A, Cutler J A, Hennekens C H, Allender P S, Walker W G, Whelton P K, Williams R R. 1998. Angiotensinogen genotype, sodium reduction, weight loss, and prevention of hypertension: trials of hypertension prevention, phase II. Hypertension 32:393-401.
Hunt S C, Geleijnse J M, Wu L L, Witteman J C, Williams R R, Grobbee D E. 1999. Enhanced blood pressure response to mild sodium reduction in subjects with the 235T variant of the angiotensinogen gene. Am J Hypertens 12:460-466.
Koivukoski L et al. 2004. Meta-analysis of genome-wide scans for hypertension and blood pressure in Caucasians shows evidence of susceptibility regions on chromosomes 2 and 3. Hum Mol Genet 13:2325-2332.
Kokubo Y et al. 2005. Association analysis between hypertension and CYBA, CLCNKB, and KCNMB1 functional polymorphisms in the Japanese population—the Suita Study. Circ J 69:138-142.
Kwok P-Y 2001. Methods for genotyping single nucleotide polymorphisms. Ann Rev Genomics Hum Genet 2:235-258.
Lifton R P, Gharavi A G, Geller D S. 2001. Molecular mechanisms of human hypertension. Cell 104:545-556.
Luft F 2003. Mendelian forms of human hypertension and mechanisms of disease. Clin Med Res 1:291-300.
Luedemann J et al. 2002. The association between behavior dependent cardiovascular risk factors and asymptomatic carotid atherosclerosis in a general population. Stroke 33: 2929-2935.
Moreno M U et al. 2006. The C242T CYBA polymorphism of NADPH oxidase is associated with essential hypertension. J Hypertens 24:1299-1306.
Nielsen P E et al. 1991. Sequence-selective recognition of DNA by strand displacement with a thymine-substituted polyamide. Science 254:1497-500.
Rabbitt P et al. 2004. The University of Manchester Longitudinal Study of Cognition in Normal Healthy Old Age, 1983 through 2003. Aging, Neuropsychol. Cogn. 11: 245-270.
Shimkets R A, Warnock D G, Bositis C M, Nelson-Williams C, Hansson J H, Schambelan M, Gill J R Jr, Ulick S, Milora R V, Findling J W, et al. 1994. Liddle's syndrome: heritable human hypertension caused by mutations in the beta subunit of the epithelial sodium channel. Cell. 79:407-414.
Staessen J A et al. 2001. Effects of three candidate genes on prevalence and incidence of hypertension in a Caucasian population. J Hypertens 19:1349-1358.
Syvanen A-C 2001. Accessing genetic variation: Genotyping single nucleotide polymorphisms. Nature Reviews Genetics 2:930-942.
Toivonen H T et al. 2000. Data mining applied to linkage disequilibrium mapping. Am. J. Hum. Genet. 67:133-45.
Turner S T et al. 2001. C825T polymorphism of the G protein beta(3)-subunit and antihypertensive response to a thiazide diuretic. Hypertension 37:739-743.
Weder A B 2007. Genetics and Hypertension. J Clin Hypertens 9:217-223
Yamamoto M et al. 2006. Interaction between serotonin 2A receptor and endothelin-1 variants in association with hypertension in Japanese. Hypertens Res 29:227-232.
Zintzaras E et al. 2006. Endothelial NO synthase gene polymorphisms and hypertension: a meta-analysis. Hypertension 48:700-710.

1. A method for risk assessment, molecular diagnosis or prognosis assessment of hypertension (HT) or a HT related condition in a mammalian subject using a biological sample obtained from the subject comprising:
a) detecting one or more HT associated biomarkers in said sample, wherein the biomarkers are related to one or more genes set forth in table 1, or said biomarkers are related to one or more polypeptides encoded by said genes, and;
c) comparing the biomarker data from the subject to biomarker data from healthy and diseased people to make risk assessment, molecular diagnosis or prognosis of HT.

2. The method according to claim 1, wherein said HT related condition comprises cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

3. The method according to claim 1, wherein at least one biomarker is a HT associated polymorphic site residing in a genomic region containing a gene set forth in table 1.

4. The method according to claim 1, wherein at least one biomarker is selected from the SNP markers set forth in tables 2 to 10.

5. The method according to claim 1, wherein at least one biomarker is a HT associated polymorphic site associated with one or more of the SNP markers set forth in tables 2 to 10.

6. The method according to claim 1, wherein at least one biomarker is a HT associated polymorphic site being in complete linkage disequilibrium with one or more of the SNP markers set forth in tables 2 to 10.

7. The method according to claim 1, wherein at least one biomarker is an expression product of a gene set forth in table 1.

8. The method according to claim 1, wherein at least one biomarker is related to biological activity or function of a polypeptide encoded by a gene set forth in table 1.

9. The method according to claim 1, wherein at least one biomarker is a metabolite of a polypeptide encoded by a gene set forth in table 1.

10. The method according to claim 1, wherein at least one biomarker is an antibody specific to a polypeptide encoded by a gene set forth in table 1.

11. The method according to claim 1, wherein said method is for identifying subjects having altered risk for developing HT or a HT related condition.

12. The method according to claim 1, wherein said method is for selecting efficient and/or safe therapy to prevent HT or a HT related condition in a subject having increased risk of HT or a HT related condition.

13. The method according to claim 1, wherein said method is for predicting efficiency or monitoring the effect of a therapy used to prevent HT or a HT related condition in a subject having increased risk of HT or a HT related condition.

14. The method according to claim 1, wherein said method is for diagnosing a subtype of HT in a subject having HT or a HT related condition.

15. The method according to claim 1, wherein said method is for selecting efficient and safe therapy to treat HT or a HT related condition in a subject having HT or a HT related condition.

16. The method according to claim 1, wherein said method is for predicting efficiency or monitoring the effect of a therapy used to treat HT or a HT related condition in a subject having HT or a HT related condition.

17. The method according to claim 1 further comprising a SNP marker set or a microsatellite marker set to assess the ancestry of a subject.

18. The method according to claim 1 further comprising a step of combining non-genetic information with the biomarker data to make risk assessment, diagnosis or prognosis of HT or a HT related condition for a subject.

19. The method according to claim 18, wherein the non-genetic information comprises age, gender, ethnicity, socioeconomic status, medical history of the subject, psychological traits and states, behavior patterns and habits, biochemical measurements, clinical measurements and family history of HT and relevant conditions.

20. The method according to claim 19, wherein the medical history of the subject comprises cerebrovascular disease, other cardiovascular disease, hypercholesterolemia, obesity, diabetes and the metabolic syndrome.

21. The method according to claim 19, wherein the relevant family history information comprises HT, cerebrovascular disease, other cardiovascular disease, hypercholesterolemia, obesity, diabetes and the metabolic syndrome.

22. The method according to claim 19, wherein the biochemical measurements comprise the measurements of determining blood, serum or plasma concentration or urinary excretion of VLDL, LDL, HDL, total cholesterol, triglycerides, apolipoprotein (a), fibrinogen, ferritin, transferrin receptor, C-reactive protein, glucose, insulin, vasoactive peptides, sodium, potassium, magnesium, calcium, selenium, saturated and unsaturated fatty acids, amino acids, dietary antioxidants such as vitamin C and E and biomarkers of alcohol intake such as gamma-glutamyltransaminase.

23. The method according to claim 19, wherein the clinical measurements comprise systolic and diastolic blood pressure measurements and measurements of obesity and adiposity comprising height, weight, body-mass index (kg/m2), waist circumference, waist-to-hip circumference ratio, skinfold thickness measurements, adipose tissue thickness measurements and measurements of amount and proportion of adipose tissue of the body.

24. The method according to claim 19, wherein the behaviour patterns and habits include tobacco smoking, physical activity, dietary intakes of nutrients, salt intake, alcohol intake and consumption patterns and coffee consumption and quality.

25. The method according to claim 1 further comprising a step of calculating the risk of HT or a HT related condition using a logistic regression equation as follows:
Risk of HT=[1+e^{-(a+Σ(bi*Xi)}]⁻¹, where e is Napier's constant, Xᵢ are variables associated with the risk of HT, bᵢ are coefficients of these variables in the logistic function, and a is the constant term in the logistic function.

26. The method according to claim 25, wherein subject's short term, median term, and/or long term risk of HT or a HT related condition is predicted.

27. A test kit for risk assessment, molecular diagnosis or prognosis assessment of HT or a HT related condition from biological samples taken from mammalian subjects comprising:
a) reagents, materials and protocols for assessing type and/or level of one or more HT associated biomarkers in a biological sample, wherein the biomarkers are related to one or more genes set forth in table 1, or said biomarkers are related to one or more polypeptides encoded by said genes, and;
b) instructions and software for comparing the biomarker data from a subject to biomarker data from healthy and diseased people to make risk assessment, molecular diagnosis or prognosis of HT or a HT related condition.

28. The test kit according to claim 27, wherein said HT related condition comprises cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

29. The test kit according to claim 27, wherein at least one biomarker is a HT associated polymorphic site residing in a genomic region containing a gene set forth in table 1.

30. The test kit according to claim 27, wherein at least one biomarker is selected from the SNP markers set forth in tables 1 to 10.

31. The test kit according to claim 27, wherein at least one biomarker is a HT associated polymorphic site associated with one or more of the SNP markers set forth in tables 2 to 10.

32. The test kit according to claim 27, wherein at least one biomarker is a HT associated polymorphic site being in complete linkage disequilibrium with one or more of the SNP markers set forth in tables 2 to 10.

33. The test kit according to claim 27, wherein at least one biomarker is an expression product of a gene set forth in table 1.

34. The test kit according to claim 27, wherein at least one biomarker is related to biological activity or function of a polypeptide encoded by a gene set forth in table 1.

35. The test kit according to claim 27, wherein at least one biomarker is a metabolite of a polypeptide encoded by a gene set forth in table 1.

36. The test kit according to claim 27, wherein at least one biomarker is an antibody specific to a polypeptide encoded by a gene set forth in table 1.

37. The test kit according to claim 27, wherein said test kit is for identifying subjects having altered risk for developing HT or a HT related condition.

38. The test kit according to claim 27, wherein said test kit is for selecting efficient and safe therapy to prevent HT or a HT related condition in a subject having increased risk of HT or a HT related condition.

39. The test kit according to claim 27, wherein said test kit is for predicting efficiency or monitoring the effect of a therapy used to prevent HT or a HT related condition in a subject having increased risk of HT or a HT related condition.

40. The test kit according to claim 27, wherein said test kit is for diagnosing a subtype of HT in a subject having HT or a HT related condition.

41. The test kit according to claim 27, wherein said test kit is for selecting efficient and safe therapy to treat HT or a HT related condition in a subject having HT or a HT related condition.

42. The test kit according to claim 27, wherein said test kit is for predicting efficiency or monitoring the effect of a therapy used to treat HT or a HT related condition in a subject having HT or a HT related condition.

43. The test kit according to claim 27 further comprising a SNP marker set or microsatellite marker set to assess the ancestry of a subject.

44. The test kit according to claim 27 further comprising a questionnaire and instructions for collecting personal and clinical information from the subject, and software and instructions for combining personal and clinical information with biomarker data to make risk assessment, diagnosis or prognosis of HT or a HT related condition.

45. The test kit according to claim 44, wherein the non-genetic information comprises age, gender, ethnicity, socioeconomic status, medical history of the subject, psychological traits and states, behavior patterns and habits, biochemical measurements, clinical measurements and family history of HT and relevant conditions.

46. The test kit according to claim 45, wherein the medical history of the subject comprises cerebrovascular disease, other cardiovascular disease, hypercholesterolemia, obesity, diabetes and the metabolic syndrome.

47. The test kit according to claim 45, wherein the relevant family history information comprises cerebrovascular disease, other cardiovascular disease, hypercholesterolemia, obesity, diabetes and the metabolic syndrome.

48. The test kit according to claim 45, wherein the biochemical measurements comprise the measurements of determining blood, serum or plasma concentration or urinary excretion of VLDL, LDL, HDL, total cholesterol, triglycerides, apolipoprotein (a), fibrinogen, ferritin, transferrin receptor, C-reactive protein, glucose, insulin, vasoactive peptides, sodium, potassium, magnesium, calcium, selenium, saturated and unsaturated fatty acids, amino acids, dietary antioxidants such as vitamin C and E and biomarkers of alcohol intake such as gamma-glutamyltransaminase.

49. The test kit according to claim 45, wherein the clinical measurements comprise systolic and diastolic blood pressure measurements and measurements of obesity and adiposity comprising height, weight, body-mass index (kg/m2), waist circumference, waist-to-hip circumference ratio, skinfold thickness measurements, adipose tissue thickness measurements and measurements of amount and proportion of adipose tissue of the body.

50. The test kit according to claim 45, wherein the behaviour patterns and habits include tobacco smoking, physical activity, dietary intakes of nutrients, salt intake, alcohol intake and consumption patterns and coffee consumption and quality.

51. The test kit according to claim 27 further comprising a step of calculating the risk of HT or a HT related condition using a logistic regression equation as follows:
Risk of HT=[1+e^{-(a+Σ(bi*Xi)}]⁻¹, where e is Napier's constant, Xᵢ are variables associated with the risk of HT, bᵢ are coefficients of these variables in the logistic function, and a is the constant term in the logistic function.

52. The test kit according to claim 27, wherein subject's short term, median term, and/or long term risk of HT or a HT related condition is predicted.

53. The test kit according to claim 27 comprising a PCR primer set for amplifying at least one of said biomarkers.

54. The test kit according to claim 27 comprising a capturing nucleic acid probe set specifically binding to at least one of said biomarkers.

55. The test kit according to claim 27 comprising a microarray or multiwell plate to assess said biomarkers.

56. Use of an agent modulating biological activity or function of a polypeptide encoded by a HT associated gene set forth in table 1 for manufacturing of a pharmaceutical composition for prevention or treatment of HT or a HT related condition in a mammalian subject

57. The use according to claim 56, wherein said HT related condition comprises cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

58. The use according to claim 56, wherein said agent enhances or reduces expression of a HT associated gene set forth in table 1.

59. The use according to claim 56, wherein said agent enhances or reduces biological activity or function of a metabolic pathway related to a HT associated gene set forth in table 1, or its encoded polypeptide.

60. The use according to claim 56, wherein said agent enhances or reduces activity of a pathophysiological pathway involved in HT or a HT related condition and related to a HT associated gene set forth in table 1, or its encoded polypeptide.

61. The use according to claim 56, wherein said agent is a recombinant polypeptide encoded by a HT associated gene set forth in table 1, or a variant, a fragment or a derivative thereof.

62. The use according to claim 56, wherein said agent is an antibody binding to a polypeptide encoded by a HT associated gene set forth in table 1.

63. The use according to claim 56, wherein said agent binds to a polypeptide encoded by a HT associated gene set forth in table 1.

64. The use according to claim 56, wherein said agent is a sequence specific gene silencing agent such as a siRNA hybridising to a RNA encoded by a HT associated gene set forth in table 1.

65. A method for preventing, treating or reducing the risk of HT or a HT related condition in a mammalian subject comprising a therapy modulating biological activity or function of a polypeptide encoded by a HT associated gene set forth in table 1.

66. The method according to claim 65, wherein said HT related condition comprises cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

67. The method according to claim 65 comprising administering to a mammalian subject in need of such treatment an effective amount of a therapeutic agent enhancing or reducing expression of a HT associated gene set forth in table 1.

68. The method according to claim 65 comprising administering to a mammalian subject in need of such treatment an effective amount of a therapeutic agent enhancing or reducing biological activity or function of a metabolic pathway related to a HT associated gene set forth in table 1, or its encoded polypeptide.

69. The method according to claim 65 comprising administering to a mammalian subject in need of such treatment an effective amount of a therapeutic agent enhancing or reducing activity of a pathophysiological pathway involved in HT or a HT related condition and related to a HT associated gene set forth in table 1, or its encoded polypeptide.

70. The method according to claim 65, wherein said therapy comprises a recombinant polypeptide encoded by a HT associated gene set forth in table 1, or a variant, a fragment or a derivative thereof.

71. The method according to claim 65, wherein said therapy comprises an antibody binding to a polypeptide encoded by a HT associated gene set forth in table 1.

72. The method according to claim 65, wherein said therapy comprises an agent binding to a polypeptide encoded by a HT associated gene set forth in table 1.

73. The method according to claim 65, wherein said therapy comprises a sequence specific gene silencing agent such as a siRNA hybridising to a RNA encoded by a HT associated gene set forth in table 1.

74. The method according to claim 65 comprising gene therapy, gene transfer, dietary treatment or a vaccination.

75. The method according to claim 74, wherein said therapy comprises the transfer of a HT associated gene set forth in table 1, or a variant, a fragment or a derivative thereof in somatic cells, in stem cells, or in affected tissues of said subject.

76. A pharmaceutical composition for preventing, treating or reducing the risk of HT or a HT related condition in a mammalian subject comprising an agent modulating biological activity or function of a polypeptide encoded by a HT associated gene set forth in table 1.

77. The pharmaceutical composition according to claim 76, wherein said HT related condition comprises cerebrovascular disease, arterial aneurysm, left ventricular hypertrophy, congestive heart failure, other congestive heart disease, coronary heart disease, other ischemic arterial disease, other arteriosclerotic disease, hypertensive renal disease or hypertensive retinal disease.

78. The pharmaceutical composition according to claim 76, wherein said agent enhances or reduces expression of a HT associated gene set forth in table 1.

79. The pharmaceutical composition according to claim 76, wherein said agent enhances or reduces biological activity or function of a metabolic pathway related to a HT associated gene set forth in table 1, or its encoded polypeptide.

80. The pharmaceutical composition according to claim 76, wherein said agent enhances or reduces activity of a pathophysiological pathway involved in HT or a HT related condition and related to a HT associated gene set forth in table 1, or its encoded polypeptide.

81. The pharmaceutical composition according to claim 76, wherein said agent is a recombinant polypeptide encoded by a HT associated gene set forth in table 1, or a variant, a fragment or a derivative thereof.

82. The pharmaceutical composition according to claim 76, wherein said agent is an antibody binding to a polypeptide encoded by a HT associated gene set forth in table 1.

83. The pharmaceutical composition according to claim 76, wherein said agent binds to a polypeptide encoded by a HT associated gene set forth in table 1.

84. The pharmaceutical composition according to claim 76, wherein said agent is a sequence specific gene silencing agent such as a siRNA hybridising to a RNA encoded by a HT associated gene set forth in table 1.

85. A method for screening agents for preventing or treating HT or a HT related condition in a mammal comprising determining the effect of an agent either on a metabolic pathway related to a polypeptide or a RNA molecule encoded by a HT associated gene set forth in table 1 in living cells; wherein an agent altering activity of a metabolic pathway is considered useful in prevention or treatment of HT or a HT related condition.

86. The method according to claim 85, wherein said agent is administered to a model system or organism, and wherein an agent altering or modulating expression, biological activity or function of a HT associated gene set forth in table 1, or its encoded polypeptide is considered useful in prevention or treatment of HT or a HT related condition.

87. The method according to claim 86, wherein the model system or organism comprises cultured microbial, insect or mammalian cells, mammalian tissues, organs or organ systems or non-human transgenic animals expressing a HT associated gene set forth in table 1.


SEQUENCE LISTING (1 sequence)

SEQ ID NO: 1 - DNA, 679 bp, Homo sapiens
acatataggtcaatctgaaaaggtggaagagaagtgaaaagcaattcttgtgctctagtcagtagtgtgtttatctttgacagccattacgtgtcaaaaattactgacccttacttaatgatatctctattgttttgggaagcctaagcagtggtaataaataggcccaataggtatcatgaatcctacatcatcgatgatcattcttgcttgcttcaccacacaggcacgtgttcccaatttgcagcaattctttgcagctattccggtgtccatgcttcttgctttttgtaaccctactatttctataatccctataatctgcactcattcataggggaggaaagaagacacagacggggcaaggccactttttgaacgcctcagcctagaaatgcgctatgccactcattctcacattctttcttctagaaatggccacacctaacagcaagggaggaaggaacacatagtctggtatgtccaggatgaagagaacataaatttaaataaacagtttgcagtctccatcacattattcrgagattaaaaatatttttctcaagtaaagatctttcttagagattagctttgaaaataaagatggtacaatatcctaaatttatttgctgcaagataattttacaatgtggccacatctgatcaggcttaataacca